(19) United States
(12) Patent Application Publication
(10) Pub. No.: US 2024/0199612 A1
(43) Pub. Date: Jun. 20, 2024

(54) IMIDAZOPYRIDINYL INHIBITORS OF PLASMA KALLIKREIN

(71) Applicant: SHIRE HUMAN GENETIC THERAPIES, INC., Lexington, MA (US)

(72) Inventors: Nikolaos PAPAIOANNOU; Jeremy Mark TRAVINS, Lexington, MA (US); Sarah Jocelyn FINK; John Mark ELLARD, Saffron Walden (GB); Alastair RAE, Saffron Walden (GB)

(21) Appl. No.: 18/550,474

(22) PCT Filed: Mar. 16, 2022
(86) PCT No.: PCT/US2022/020474

§ 371 Date: Sep. 14, 2023

Related U.S. Application Data
(60) Provisional application No. 63/152,494, filed on Feb. 23, 2021.

(51) Int. Cl. C07D 471/04; A61K 31/437; A61K 31/506

(52) U.S. Cl. CPC C07D 471/04; A61K 31/437; A61K 31/506

(57) ABSTRACT

The present invention provides compounds and compositions thereof which are useful as inhibitors of plasma kallikrein and which exhibit desirable characteristics for the same.

### CROSS REFERENCE TO RELATED APPLICATIONS

This application claims the benefit of U.S. Provisional Application No. 63/162,494, filed Mar. 17, 2021, which is herein incorporated by reference in its entirety.

### I. BACKGROUND OF THE INVENTION

Plasma Kallikrein (PKa) is a serine protease zymogen in blood that is converted to its catalytically active form by coagulation factor XIIa, and contributes to the innate inflammatory response and intrinsic cascade of blood coagulation. The mechanisms that lead to the activation of this pathway in vivo include interactions with polyphosphates released from activated platelets and deficiency of C1 inhibitor (C1-INH), the primary physiological inhibitor of PKa. PKa-mediated cleavage of high-molecular weight kininogen generates the potent vasodilator and pro-inflammatory nonapeptide bradykinin (BK), which activates the bradykinin 2 receptor. Subsequent cleavage of BK by carboxypeptidases generates des-Arg9-BK, which activates the B1 receptor. Both B1 and B2 receptors are expressed by vascular, glial, and neuronal cell types, with the highest levels of retinal expression detected in the ganglion cell layer and inner and outer nuclear layers. Activation of B1 and B2 receptors causes vasodilation and increases vascular permeability.

PKa is also associated with a number of disorders, such as hereditary angioedema (HAE), an autosomal dominant disease characterized by painful, unpredictable, recurrent attacks of inflammation affecting the hands, feet, face, abdomen, urogenital tract, and the larynx. Prevalence for HAE is uncertain but is estimated to be approximately 1 case per 50,000 persons without known differences among ethnic groups. HAE is caused by deficient (Type I) or dysfunctional (Type II) levels of C1-INH, which inhibits PKa, bradykinin, and other serine proteases in the blood. Individuals with hereditary angioedema (HAE) are deficient in C1-INH and consequently undergo excessive bradykinin generation, which in turn cause painful, debilitating, and potentially fatal swelling attacks. If left untreated, HAE can result in a mortality rate as high as 40% primarily due to upper airway obstruction.

### II. SUMMARY OF THE INVENTION

The present disclosure is based on, at least in part, the development of a number of compounds which bind to plasma kallikrein and effectively inhibit its activity. Accordingly, provided herein are compounds and uses thereof for targeting plasma kallikrein and/or treating plasma kallikrein-mediated diseases and disorders, novel intermediates, and processes for preparing compounds disclosed herein. The disclosure also extends to pharmaceutical compositions comprising any one of the same, and use of compounds or compositions herein for treatment, in particular treatment of autoimmune disease, such as HAE.

In some embodiments, the present invention provides a compound of Formula (I):

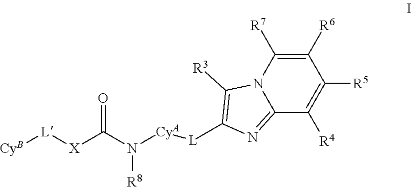

or a pharmaceutically acceptable salt thereof, wherein each of Cy^{A}, Cy^{B}, L, L′, R³, R⁴, R⁵, R⁶, R⁷, and R⁸ is defined and described in classes and subclasses herein, both singly and in combination. In certain embodiments, the present invention provides compounds of Formulae (I)-(VI-c), as defined and described in classes and subclasses herein.

In some embodiments, the present invention also provides methods of using compounds of Formulae (I)-(VI-c).

Advantageously, compounds of the present disclosure have therapeutic activity and adequate levels of bioavailability and/or adequate half-life for use as a therapeutic.

### III. DETAILED DESCRIPTION OF CERTAIN EMBODIMENTS

A. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The abbreviations used herein have their conventional meaning within the chemical and biological arts. The chemical structures and formulae set forth herein are constructed according to the standard rules of chemical valency known in the chemical arts.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In some embodiments, aliphatic groups contain 1-4 aliphatic carbon atoms. In some embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocyclyl” or “cycloalkyl”) refers to a monocyclic C₃-C₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “halogen” means F, Cl, Br, or I.

The term “aryl” refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In some embodiments, an 8-10 membered bicyclic aryl group is an optionally substituted naphthyl ring. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-” refer to groups having 5 to 10 ring atoms, preferably 5, 6, or 9 ring atoms; having 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (or in the case of a bivalent fused heteroarylene ring system, at least one radical or point of attachment is on a heteroaromatic ring). Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted.

As used herein, the terms “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in this context in reference to a ring atom, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As used herein and unless otherwise specified, the suffix “-ene” is used to describe a bivalent group. Thus, any of the terms above can be modified with the suffix “-ene” to describe a bivalent version of that moiety. For example, a bivalent carbocycle is “carbocycylene”, a bivalent aryl ring is “arylene”, a bivalent benzene ring is “phenylene”, a bivalent heterocycle is “heterocyclylene”, a bivalent heteroaryl ring is “heteroarylene”, a bivalent alkyl chain is “alkylene”, a bivalent alkenyl chain is “alkenylene”, a bivalent alkynyl chain is “alkynylene”, and so forth.

As described herein, compounds of the invention may, when specified, contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.

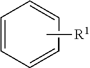

refers to at least

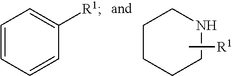

refers to at least

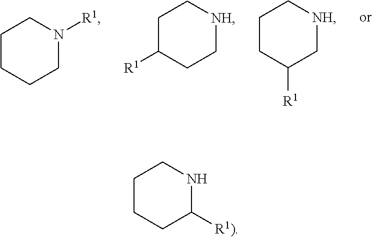

In addition, in a polycyclic ring system, substituents may, unless otherwise indicated, replace a hydrogen on any individual ring (e.g.,

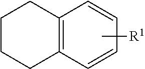

refers to at least

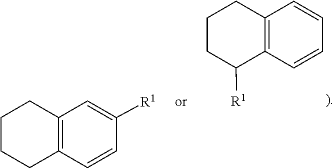

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O(CH₂)₀₋₄C(O)OR^{∘}; —O(CH₂)₀₋₄OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)_{0- 4}C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6 membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12 membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{∀}, -(haloR^{∀}), —(CH₂)₀₋₂OH, —(CH₂)₀₋₂OR^{∀}, —(CH₂)₀₋₂CH(OR^{∀})₂; —O(haloR^{∀}), —CN, —N₃, —(CH₂)₀₋₂C(O)R^{∀}, —(CH₂)₀₋₂C(O)OH, —(CH₂)₀₋₂C(O)OR^{∀}, —(CH₂)₀₋₂SR^{∀}, —(CH₂)₀₋₂SH, —(CH₂)₀₋₂NH₂, —(CH₂)₀₋₂NHR^{∀}, —(CH₂)₀₋₂ NR•₂, —NO₂, —SiR^{∀}₃, —OSiR^{∀}₃, —C(O)SR^{∀}, —(C₁₋₄ straight or branched alkylene)C(O)OR^{∀}, or —SSR^{∀} wherein each R^{∀} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6 membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR•), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6 membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{•}, —C(O)ORT, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(RT)S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12 membered saturated, partially unsaturated, or aryl mono- or bicyclic ring having 0-4 heteroatoms independently selected from nitrogen, oxygen, or sulfur.

Suitable substituents on the aliphatic group of R are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference.

In certain embodiments, the neutral forms of the compounds are regenerated by contacting the salt with a base or acid and isolating the parent compound in the conventional manner. In some embodiments, the parent form of the compound differs from the various salt forms in certain physical properties, such as solubility in polar solvents.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In some embodiments, compounds of the present disclosure are provided as a single enantiomer or single diastereoisomer. Single enantiomer refers to an enantiomeric excess of 80% or more, such as 81, 82, 83, 84, 85, 86, 87, 88, 89, 90, 91, 92, 93, 94, 95, 96, 97, 98 or 99%. Single diastereoisomer excess refers to an excess of 80% or more, for example 81, 82, 83, 84, 85, 86, 87, 88, 89, 90, 91, 92, 93, 94, 95, 96, 97, 98 or 99%.

The term “oxo,” as used herein, means an oxygen that is double bonded to a carbon atom, thereby forming a carbonyl.

The symbol “ [symbol] ”, except when used as a bond to depict unknown or mixed stereochemistry, denotes the point of attachment of a chemical moiety to the remainder of a molecule or chemical formula.

The articles “a” and “an” are used herein to refer to one or to more than one (i.e., to at least one) of the grammatical object of the article. By way of example, “an element” means one element or more than one element.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses.

As will be understood from context, a “reference” compound is one that is sufficiently similar to a particular compound of interest to permit a relevant comparison. In some embodiments, information about a reference compound is obtained simultaneously with information about a particular compound. In some embodiments, information about a reference compound is historical. In some embodiments, information about a reference compound is stored, for example in a computer-readable medium. In some embodiments, comparison of a particular compound of interest with a reference compound establishes identity with, similarity to, or difference of the particular compound of interest relative to the compound.

As used herein, the phrase “therapeutic agent” refers to any agent that has a therapeutic effect and/or elicits a desired biological and/or pharmacological effect, when administered to a subject.

As used herein, the term “therapeutically effective amount” refers to an amount of a therapeutic agent that confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic agent effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific therapeutic agent employed; the specific composition employed; the age, body weight, general health, sex and diet of the subject; the time of administration, route of administration, and/or rate of excretion or metabolism of the specific therapeutic agent employed; the duration of the treatment; and like factors as is well known in the medical arts.

As used herein, the term “treatment” (also “treat” or “treating”) refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

B. Compounds

In some embodiments, a provided compound is of Formula I.

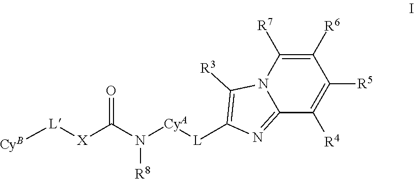

or a pharmaceutically acceptable salt thereof, wherein

Cy^{A} is a phenylene or 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4 —R^{A} groups;
each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur;

each R is independently hydrogen or an optionally substituted C₁₋₆ aliphatic group;

Cy^{B} is selected from phenyl, 8- to 10-membered bicyclic aryl, 5- to 6-membered heteroaryl having 1-3 heteroatoms independently selected from oxygen, nitrogen, and sulfur or 7- to 10-membered heteroaryl having 1-4 heteroatoms independently selected from oxygen, nitrogen, and sulfur, wherein Cy^{B} is substituted with 0-5 —R^{B} groups;
each R^{B} is independently selected from oxo, halogen, haloalkyl, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;

L is an optionally substituted C₁₋₃ hydrocarbon chain, wherein 1 to 3 methylene units are optionally and independently replaced with —O—, —NR^{z}—, —S—, —SO—, or —SO₂—; or L is an optionally substituted 5- to 6-membered saturated or partially unsaturated heterocyclene, having 1-3 heteroatoms independently selected from oxygen, nitrogen, and sulfur;
each R^{z} is independently selected from hydrogen, —(CH₂)₀₋₃OR, —(CH₂)₀₋₃C(O)OR, or an optionally substituted C₁₋₆ aliphatic group;

X is —O— or —NR^{y}—;

R^{y} is hydrogen or an optionally substituted C₁₋₆ aliphatic group;

L′ is a covalent bond or an optionally substituted C₁₋₃ hydrocarbon chain, wherein a carbon of L′ may optionally be taken together with R^{y} to form a 3- to 7-membered heterocyclic ring;

each R³, R⁴, R⁵, R⁶, and R⁷ is independently selected from hydrogen or -L^{C}-R^{C}, wherein
each L^{C} is independently selected from a covalent bond or an optionally substituted C₁₋₆ hydrocarbon chain, wherein 1 to 3 methylene units are optionally and independently replaced with —O— or —NR—;
each R^{C} is independently selected from halogen, haloalkyl, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{C}, or an optionally substituted group selected from C₁₋₆ aliphatic;
   each Cy^{C} is independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{C} is substituted with 0-4 -L^{D}-R^{D} groups;
   each L^{D} is independently selected from a covalent bond or an optionally substituted C₁₋₆ hydrocarbon chain, wherein 1 to 3 methylene units are optionally and independently replaced with —O— or —NR—;
   each R^{D} is independently selected from oxo, halogen, haloalkyl, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur; and
R⁸ is selected from hydrogen, —OR, or an optionally substituted C₁₋₆ aliphatic group.

It will be appreciated that, “oxo” refers a double bonded oxygen substitution on a carbon “C═O”, where the carbon atom is part of the structure or group that is substituted by oxo. For example, where Cy^{C} is substituted with -L^{D}-R^{D}, and where L^{D} is a covalent bond and R^{D} is oxo, the carbon atom substituted with oxo (i.e., the carbon in C═O) is part of Cy^{C} (e.g., a structure of Cy^{C} being cyclopentyl substituted with -L^{D}-R^{D} at the 2-position, where L^{D} is a covalent bond and R^{D} is oxo corresponds to

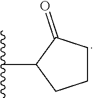 [SMILES: *C1CCCC1=O]

In some embodiments, Cy^{A} is a phenylene, wherein Cy^{A} is substituted with 0-4 —R^{A} groups. In some embodiments, Cy^{A} is a phenylene, wherein Cy^{A} is substituted with 0-2 —R groups.

In some embodiments, Cy^{A} is a 5- to 6-membered monocyclic heteroarylene having 1-3 heteroatoms independently selected from oxygen, nitrogen, and sulfur, wherein Cy^{A} is substituted with 0-4 —R^{A} groups.

In some embodiments, Cy^{A} is a 6-membered monocyclic heteroarylene having 1-3 heteroatoms independently selected from oxygen, nitrogen, and sulfur, wherein Cy^{A} is substituted with 0-4 —R^{A} groups. In some embodiments, Cy^{A} is a 6-membered monocyclic heteroarylene having 1-3 nitrogen heteroatoms, wherein Cy^{A} is substituted with 0-4 R^{A} groups. In some embodiments, Cy^{A} is a pyridinediyl substituted with 0-1 R^{A} groups. In some embodiments, Cy^{A} is a pyrimidinediyl substituted with 0-1 R^{A} groups. In some embodiments, Cy^{A} is a pyridazinediyl substituted with 0-1 R^{A} groups. In some embodiments, Cy^{A} is a triazinediyl substituted with 0-1 R^{A} groups. In some embodiments, Cy^{A} is unsubstituted pyridinediyl. In some embodiments, Cy^{A} is unsubstituted pyrimidinediyl.

In some embodiments, Cy^{A} is a 5-membered monocyclic heteroarylene having 1-3 heteroatoms independently selected from oxygen, nitrogen, and sulfur, wherein Cy^{A} is substituted with 0-2 —R^{A} groups. In some embodiments, Cy^{A} is an unsubstituted thiadiazolediyl. In some embodiments, Cy^{A} is an unsubstituted oxadiazolediyl. In some embodiments, Cy^{A} is an unsubstituted triazolediyl.

In some embodiments, Cy^{A} is a 7- to 12-membered bicyclic heteroarylene having 1-4 heteroatoms independently selected from oxygen, nitrogen, and sulfur, wherein Cy^{A} is substituted with 0-4 —R^{A} groups. In some embodiments, Cy^{A} is a 9-membered bicyclic heteroarylene having 3-4 heteroatoms independently selected from oxygen and nitrogen, wherein Cy^{A} is substituted with 0-1 —R^{A} groups. In some embodiments, Cy^{A} is a 10-membered bicyclic heteroarylene having 3-4 heteroatoms independently selected from oxygen and nitrogen, wherein Cy^{A} is substituted with 0-1 —R^{A} groups. In some embodiments, Cy^{A} is triazolopyridinediyl substituted with 0-1 —R^{A} groups. In some embodiments, Cy^{A} is imidazopyridinediyl substituted with 0-1 —R^{A} groups. In some embodiments, Cy^{A} is triazolopyrazinediyl substituted with 0-1 —R^{A} groups.

In some embodiments, Cy^{A} is selected from the group consisting of:

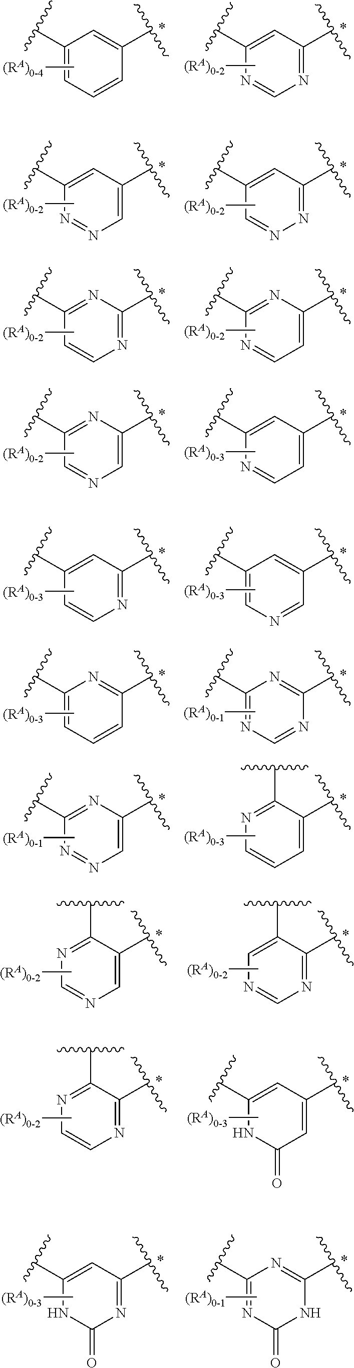

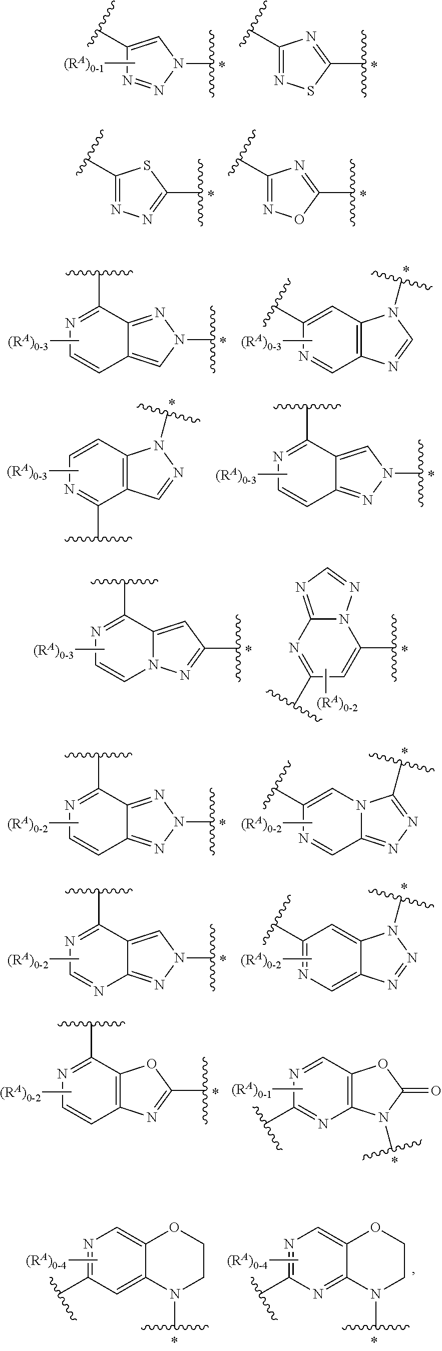

wherein * represents point of attachment to L.

In some embodiments, Cy^{A} is selected from the group consisting of:

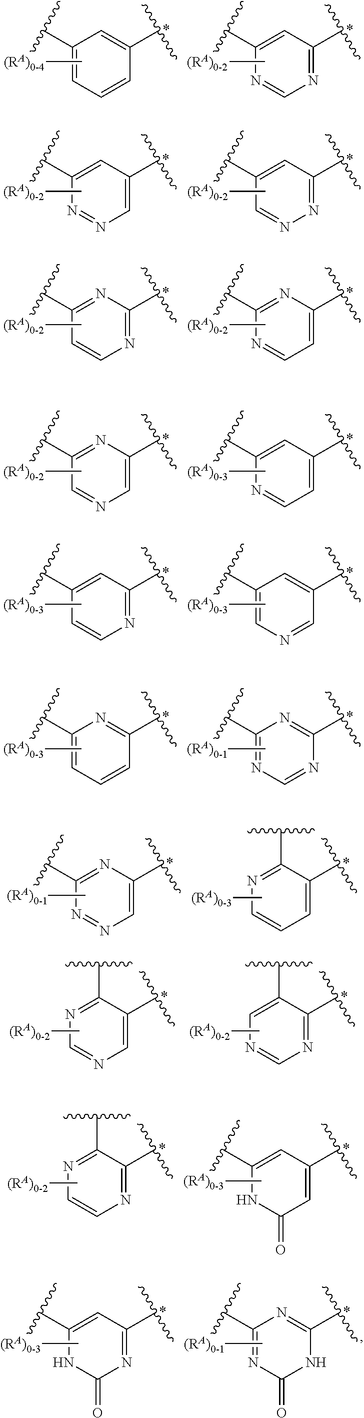

wherein * represents the point of attachment to L.

In some embodiments, Cy^{A} is selected from the group consisting of:

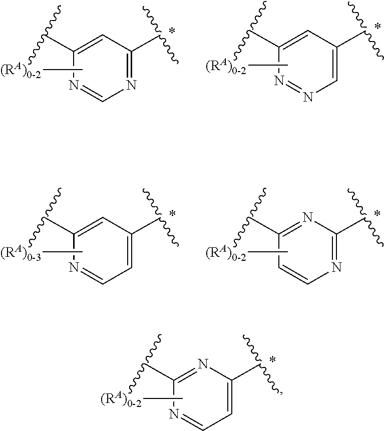

wherein * represents the point of attachment to L.

In some embodiments, Cy^{A} is selected from the group consisting of:

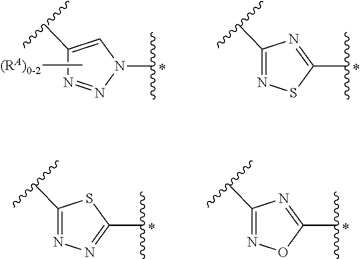

wherein * represents the point of attachment to L.

In some embodiments, each R^{A} is independently selected from oxo, halogen, —CN, —C(O)₂R, —N(R)₂, —OR, —SR, —S(O)R, —S(O)₂R, or an optionally substituted group selected from C₁₋₆ aliphatic, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur.

In some embodiments, substituents on an optionally substituted R^{A} group are independently halogen, —(CH₂)₀₋₄OR^{∘}, or —(CH₂)₀₋₄N(R^{∘})₂, wherein each R^{∘} is independently as defined above and described in classes and subclasses herein.

In some embodiments, a single instance of R^{A} is oxo. In some embodiments, a single instance of R^{A} is halogen. In some embodiments, a single instance of R^{A} is —CN. In some embodiments, a single instance of R^{A} is —C(O)₂R. In some embodiments, a single instance of R^{A} is —N(R)₂. In some embodiments, a single instance of R^{A} is —OR. In some embodiments, a single instance of R^{A} is —SR. In some embodiments, a single instance of R^{A} is —SR, wherein R is optionally substituted C₁₋₆ aliphatic. In some embodiments, a single instance of R^{A} is —S(O)R. In some embodiments, a single instance of R^{A} is —S(O)R, wherein R is optionally substituted C₁₋₆ aliphatic. In some embodiments, a single instance of R^{A} is —S(O)₂R. In some embodiments, a single instance of R^{A} is —S(O)₂R, wherein R is optionally substituted C₁₋₆ aliphatic.

It will be appreciated that references herein to embodiments in which “a single instance” of a substituent is defined are not limited to monosubstituted embodiments. For example, “[i]n some embodiments, a single instance of R^{A} is oxo” includes embodiments in which at least one instance of R^{A} is oxo and which may comprise one or more additional R^{A} groups as defined herein.

In some embodiments, a single instance of R^{A} is C₁₋₆ aliphatic substituted with halogen. In some embodiments, a single instance of R^{A} is C₁₋₆ aliphatic substituted with —(CH₂)₀₋₄OR^{∘}, wherein R^{∘} is selected from hydrogen or C₁₋₆ aliphatic. In some embodiments, a single instance of R^{A} is C₁₋₆ aliphatic substituted with —(CH₂)₀₋₄N(R^{∘})₂, wherein each R^{∘} is independently selected from hydrogen or C₁₋₆ aliphatic.

In some embodiments, a single instance of R^{A} is optionally substituted 3- to 7-membered saturated or partially unsaturated monocyclic carbocyclyl. In some embodiments, a single instance of R^{A} is optionally substituted cyclopropyl.

In some embodiments, a single instance of R^{A} is optionally substituted 3- to 7-membered saturated or partially unsaturated monocyclic heterocyclyl having 1-2 heteroatoms selected from oxygen, nitrogen, or sulfur. In some embodiments, a single instance of R^{A} is optionally substituted 3- to 7-membered saturated monocyclic heterocyclyl having 1-2 heteroatoms selected from oxygen and nitrogen. In some embodiments, a single instance of R^{A} is optionally substituted oxetanyl. In some embodiments, a single instance of R^{A} is oxetanyl optionally substituted with halogen or —(CH₂)₀₋₄OR^{∘}. In some embodiments, a single instance of R^{A} is pyrrolidinyl.

In some embodiments, Cy^{B} is selected from phenyl, a 5- to 6-membered heteroaryl having 1-3 heteroatoms independently selected from oxygen, nitrogen, and sulfur or a 7- to 10-membered heteroaryl having 1-4 heteroatoms independently selected from oxygen, nitrogen, and sulfur, wherein Cy^{B} is substituted with 0-4 —R^{B} groups.

In some embodiments, Cy^{B} is selected from phenyl and a 5- to 6-membered heteroaryl having 1-3 heteroatoms independently selected from oxygen, nitrogen, and sulfur, wherein Cy^{B} is substituted with 0-4 —R^{B} groups.

In some embodiments, Cy^{B} is phenyl, wherein Cy^{B} is substituted with 0-4 —R^{B} groups. In some embodiments, Cy^{B} is phenyl, wherein Cy^{B} is substituted with 0-3 —R^{B} groups.

In some embodiments, Cy^{B} is a 6-membered heteroaryl having 1-3 heteroatoms independently selected from oxygen, nitrogen, and sulfur, wherein Cy^{B} is substituted with 0-4 —R^{B} groups. In some embodiments, Cy^{B} is a 6-membered heteroaryl having 1-3 nitrogens, wherein Cy^{B} is substituted with 0-4 —R^{B} groups. In some embodiments, Cy^{B} is a pyrimidinyl group substituted with 0-2 —R^{B} groups. In some embodiments, Cy^{B} is a pyridinyl group substituted with 0-2 —R^{B} groups. In some embodiments, Cy^{B} is a pyrazinyl group substituted with 0-1 —R^{B} groups. In some embodiments, Cy^{B} is a pyridazinyl group substituted with 0-1 —R^{B} groups. In some embodiments, Cy^{B} is a 1,3,5-triazinyl group substituted with 0-1 —R^{B} groups.

In some embodiments, Cy^{B} is a 5-membered heteroaryl having 1-3 heteroatoms independently selected from oxygen, nitrogen, and sulfur, wherein Cy^{B} is substituted with 0-4 —R^{B} groups. In some embodiments, Cy^{B} is a 5-membered heteroaryl having 1-2 heteroatoms independently selected from sulfur and nitrogen, wherein Cy^{B} is substituted with 0-4 —R^{B} groups. In some embodiments, Cy^{B} is a thienyl group substituted with 0-2 —R^{B} groups. In some embodiments, Cy^{B} is a thiazolyl group substituted with 0-1 —R^{B} groups. In some embodiments, Cy^{B} is a thiadiazolyl group substituted with 0-1 —R^{B} groups.

In some embodiments, Cy^{B} is selected from the group consisting of:

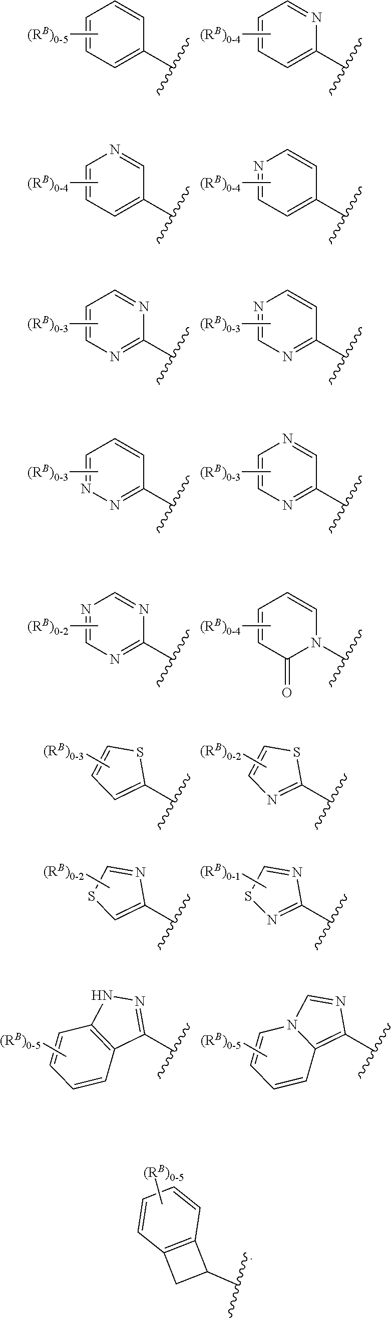

In some embodiments, Cy^{B} is selected from the group consisting of:

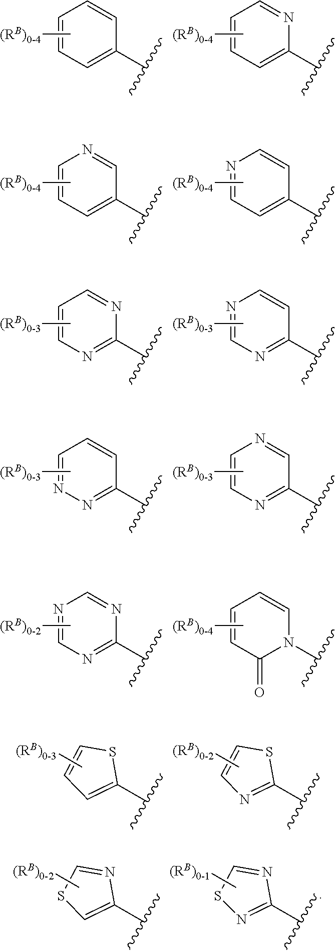

In some embodiments, Cy^{B} is selected from the group consisting of:

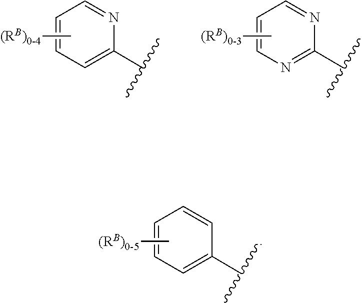

In some embodiments, each R^{B} is independently selected from oxo, halogen, —CN, —NO₂, —N(R)₂, —N(R)C(O)₂R, —OR, or an optionally substituted group selected from C₁₋₆ aliphatic or a 5-membered heteroaryl having 1-4 heteroatoms independently selected from oxygen, nitrogen, and sulfur. In some embodiments, each R^{B} is independently selected from halogen, —OR, or an optionally substituted 5-membered heteroaryl having 1-4 heteroatoms independently selected from oxygen, nitrogen, and sulfur.

In some embodiments, substituents on an optionally substituted R^{B} group are independently selected from oxo, halogen, and —(CH₂)₀₋₄OR^{∘}, wherein R^{∘} is as defined above and described in classes and subclasses herein.

In some embodiments, a single instance of R^{B} is oxo. In some embodiments, a single instance of R^{B} is halogen. In some embodiments, a single instance of R^{B} is fluorine. In some embodiments, a single instance of R^{B} is chlorine. In some embodiments, a single instance of R^{B} is —CN. In some embodiments, a single instance of R^{B} is —NO₂. In some embodiments, a single instance of R^{B} is —N(R)₂, In some embodiments, a single instance of R^{B} is —N(R)C(O)₂R. In some embodiments, a single instance of R^{B} is —OR. In some embodiments, a single instance of R^{B} is —OMe.

In some embodiments, a single instance of R^{B} is optionally substituted C₁₋₆ aliphatic. In some embodiments, a single instance of R^{B} is C₁₋₆ aliphatic substituted with halogen.

In some embodiments, a single instance of R^{B} is —N(R)C(O)₂R, wherein each R is independently selected from hydrogen or C₁₋₆ aliphatic optionally substituted with —(CH₂)₀₋₄R^{∘}.

In some embodiments, a single instance of R^{B} is —OR, wherein each R is independently selected from hydrogen or C₁₋₆ aliphatic optionally substituted with halogen, —(CH₂)₀₋₄OR^{∘}, or (CH₂)₀₋₄C(O)OR^{∘}.

In some embodiments, a single instance of R^{B} is a 5-membered heteroaryl having 1-4 heteroatoms independently selected from oxygen, nitrogen, and sulfur. In some embodiments, a single instance of R^{B} is tetrazolyl.

In some embodiments, L is an optionally substituted C₁₋₃ hydrocarbon chain, wherein 1-3 methylene units are optionally replaced with —O—, —NR^{z}—, —S—, or —SO₂—. In some embodiments, L is an optionally substituted C₁₋₃ hydrocarbon chain, wherein 1 methylene unit is optionally replaced with —O— or —NR^{z}—.

In some embodiments, L is an optionally substituted C₁ hydrocarbon chain.

In some embodiments, L is an optionally substituted C₁ hydrocarbon chain, wherein the 1 methylene unit is replaced with 5-membered saturated or partially unsaturated heterocyclene having 1 nitrogen heteroatom, optionally substituted with —(CH₂)₀₋₄OR^{∘}, wherein R^{∘} is as defined above and described in classes and subclasses herein.

In some embodiments, L is —CH₂—. In some embodiments, L is optionally substituted

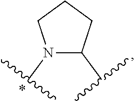 [SMILES: *C1CCCN1*]

wherein * represents the point of attachment to Cy^{A}. In some embodiments, L is optionally substituted

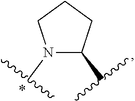 [SMILES: *[C@@H]1CCCN1*]

wherein * represents the point of attachment to Cy^{A}. In some embodiments, L is optionally substituted

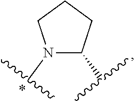 [SMILES: *[C@H]1CCCN1*]

wherein * represents the point of attachment to Cy^{A}. In some embodiments, L is

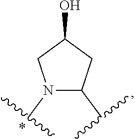 [SMILES: *C1C[C@H](O)CN1*]

wherein * represents the point of attachment to Cy^{A}. In some embodiments, L is

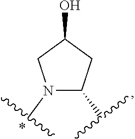 [SMILES: *[C@H]1C[C@H](O)CN1*]

wherein * represents the point of attachment to Cy^{A}. In some embodiments, L is

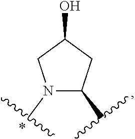 [SMILES: *[C@@H]1C[C@H](O)CN1*]

wherein * represents the point of attachment to Cy^{A}.

In some embodiments, L is an optionally substituted C₂ hydrocarbon chain, wherein 1 methylene unit is optionally replaced with —NR^{z}— or —O—. In some embodiments, L is an optionally substituted C₂ hydrocarbon chain, wherein the methylene unit connected to Cy^{A} is replaced with —NR^{z}— or —O—. In some embodiments, L is an optionally substituted C₂ hydrocarbon chain, wherein the methylene unit connected to Cy^{A} is replaced with —NR^{z}—. In some embodiments, L is an optionally substituted C₂ hydrocarbon chain, wherein the methylene unit connected to Cy^{A} is replaced with —O—. In some embodiments, L is *—NHCH(Me)-, wherein * represents the point of attachment to Cy^{A}. In some embodiments, L is

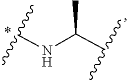 [SMILES: *N[C@H](*)C]

wherein * represents the point of attachment to Cy^{A}. In some embodiments, L is

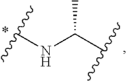 [SMILES: *N[C@@H](*)C]

wherein * represents the point of attachment to Cy^{A}. In some embodiments, L is *—NHCH₂—, wherein * represents the point of attachment to Cy^{A}. In some embodiments, L is *N(CH₃)CH₂—, wherein * represents the point of attachment to Cy^{A}. In some embodiments, L is

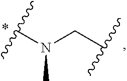

wherein * represents the point of attachment to Cy^{A}. In some embodiments, L is

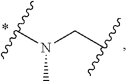

wherein * represents the point of attachment to Cy^{A}. In some embodiments, L is *—OCH(Me)-, wherein * represents the point of attachment to Cy^{A}. In some embodiments, L is *—OCH₂—, wherein * represents the point of attachment to Cy^{A}. In some embodiments, L comprises a two-atom spacer between Cy^{A} and

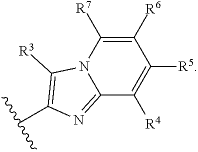

In some embodiments, L is an optionally substituted 5- to 6-membered saturated or partially unsaturated heterocyclene, having 1-3 heteroatoms independently selected from oxygen, nitrogen, and sulfur. In some embodiments, L is an optionally substituted 5-membered saturated or partially unsaturated heterocyclene, having 1 heteroatom independently selected from oxygen, nitrogen, and sulfur. In some embodiments, L is an optionally substituted pyrrolidinediyl group.

In some embodiments, optional substituents on L are independently selected from —(CH₂)₀₋₄R^{∘}, —(CH₂)₀₋₄OR^{∘}, —(CH₂)₀₋₄OC(O)R^{∘}, and —(CH₂)₀₋₄N(R^{∘})₂, wherein each R^{∘} is independently as defined above and described in classes and subclasses herein.

In some embodiments, X is —O—. In some embodiments, X is —NR^{y}—.

In some embodiments, R^{y} is hydrogen. In some embodiments, R^{y} is an optionally substituted C₁₋₆ aliphatic group.

In some embodiments, L′ is a covalent bond. In some embodiments, L′ is an optionally substituted C₁₋₃ hydrocarbon chain. In some embodiments, L′ is —CH₂—. In some embodiments, L′ is —CH(CH₃)—. In some embodiments, L′ is

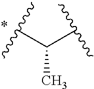

wherein * represents the point of attachment to Cy^{B}. In some embodiments, L′ is

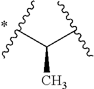

wherein * represents the point of attachment to Cy^{B}. In some embodiments, L′ is a methylene unit optionally substituted with —(CH₂)₀₋₄R^{∘} or —(CH₂)₀₋₄OR^{∘}, wherein R^{∘} is independently as defined above and described in classes and subclasses herein. In some embodiments, R^{∘} is hydrogen or C₁₋₆ aliphatic.

In certain embodiments, a carbon of L′ may optionally be taken together with R^{z} to form a 3- to 7-membered heterocyclic ring. In certain embodiments, a carbon of L′ may optionally be taken together with R^{y} to form a 4-membered heterocyclic ring.

In some embodiments, each of R³, R⁴, R⁵, R⁶, and R⁷ is independently selected from hydrogen or L^{C}-R^{C}, wherein each L^{C} is independently selected from a covalent bond or an optionally substituted C₁₋₆ hydrocarbon chain, wherein 1 to 3 methylene units are optionally and independently replaced with —O— or —NR—; and wherein each R^{C} is independently selected from halogen, —CN, —C(O)R, —C(O)₂R, —C(O)N(R)₂, —N(R)₂, —N(R)C(O)R, —N(R)C(O)₂R, —N(R)S(O)₂R, —S(O)₂R, —S(O)₂N(R)₂, Cy^{C}, or an optionally substituted group selected from C₁₋₆ aliphatic.

In some embodiments, R³ is selected from hydrogen or L^{C}-R^{C}, wherein L^{C} is a covalent bond and R^{C} is halogen. In some embodiments, R³ is hydrogen.

In some embodiments, R⁴ is selected from hydrogen or L^{C}-R^{C}, wherein L^{C} is selected from a covalent bond or an optionally substituted C₁₋₆ hydrocarbon chain, wherein 1 to 3 methylene units are optionally and independently replaced with —O— or —NR—; and wherein R^{C} is selected from halogen, —CN, —C(O)R, —C(O)₂R, —C(O)N(R)₂, —N(R)₂, —N(R)C(O)R, —N(R)C(O)₂R, —N(R)S(O)₂R, —OR, —S(O)₂R, —S(O)₂N(R)₂, Cy^{C}, or an optionally substituted group selected from C₁₋₆ aliphatic.

In some embodiments, R⁴ is selected from hydrogen or L^{C}-R^{C}, wherein L^{C} is a covalent bond and wherein R^{C} is selected from halogen, —CN, —C(O)R, —C(O)₂R, —C(O)N(R)₂, —N(R)₂, —N(R)C(O)R, —N(R)C(O)₂R, —N(R)S(O)₂R, —OR, —S(O)₂R, —S(O)₂N(R)₂, Cy^{C}, or an optionally substituted group selected from C₁₋₆ aliphatic. In some embodiments, R⁴ is hydrogen.

In some embodiments, R⁴ is selected from the group consisting of:

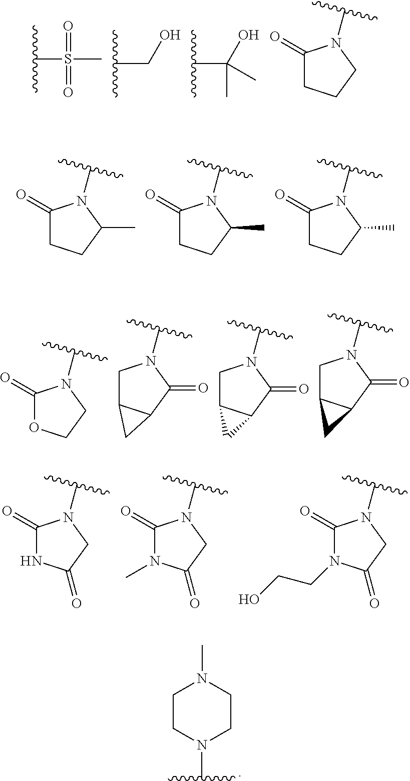

In some embodiments of R⁴, optional substituents on a C₁₋₆ aliphatic group are selected from —(CH₂)₀₋₄R^{∘}, —(CH₂)₀₋₄OR^{∘}, —CN, —(CH₂)₀₋₄N(R^{∘})₂, and —(CH₂)₀₋₄C(O)OR^{∘}, wherein each R^{∘} is independently as defined above and described in classes and subclasses herein.

In some embodiments of R⁴, Cy^{C} is selected from a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{C} is substituted with 0-4 -L^{D}-R^{D} groups. In some embodiments of R⁴, Cy^{C} is a 5-membered saturated or partially unsaturated monocyclic heterocyclyl having 1-2 heteroatoms selected from oxygen, nitrogen, or sulfur.

In some embodiments of R⁴, Cy^{C} is selected from the group consisting of:

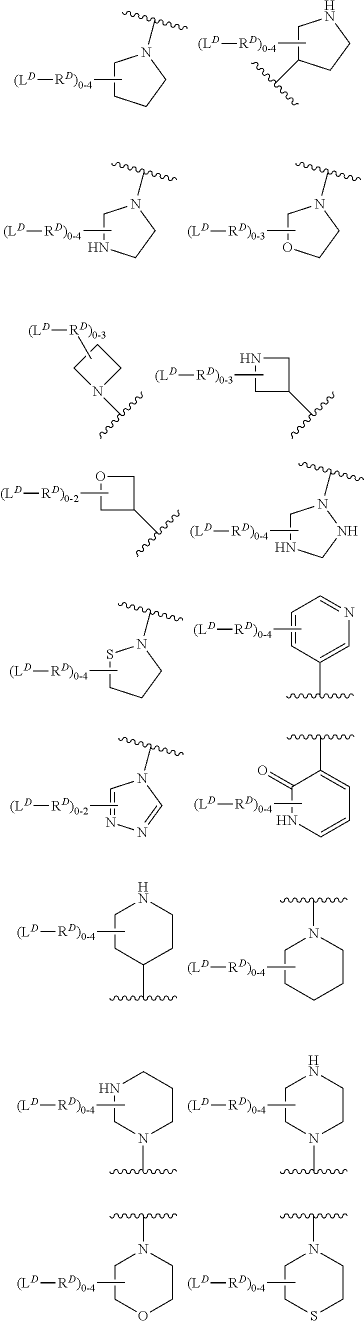

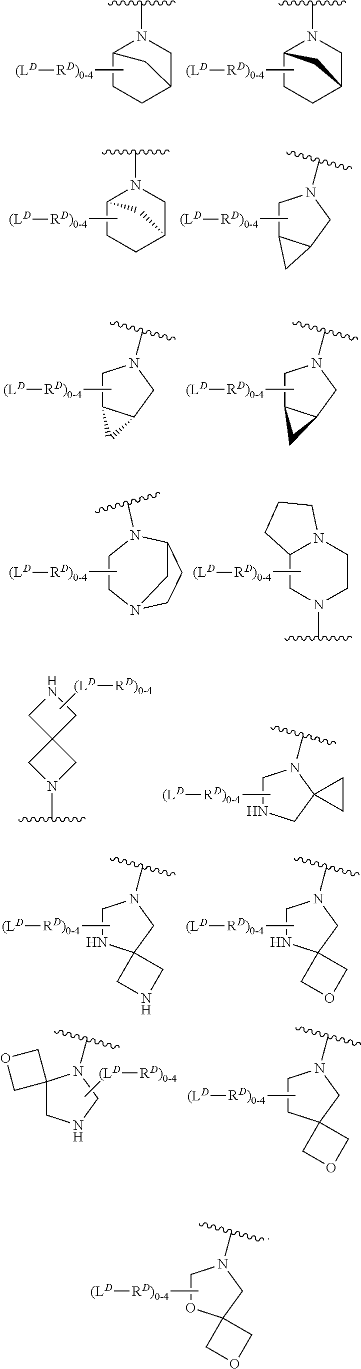

In some embodiments of R⁴, R^{D} is selected from oxo, halogen, —C(O)₂R, —N(R)₂, —OR, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur.

In some embodiments of a R^{D} group of R⁴, optional substituents on R^{D} are selected from halogen, —(CH₂)₀₋₄R^{∘}, —(CH₂)₀₋₄OR^{∘}, —(CH₂)₀₋₄N(R^{∘})₂, —(CH₂)₀₋₄C(O)OR^{∘}, and —OP(O)(OR^{∘})₂, wherein each R^{∘} is independently as defined above and described in classes and subclasses herein.

In some embodiments of R⁴, L^{D} is a covalent bond.

In some embodiments, R⁵ is hydrogen.

In some embodiments, R⁵ is L^{C}-R^{C}, wherein L^{C} is a covalent bond and R^{C} is Cy^{C}. In some embodiments, Cy^{C} is a cyclopropyl group.

In some embodiments, R⁶ is selected from hydrogen or L^{C}-R^{C}, wherein L^{C} is a covalent bond, and wherein R^{C} is selected from halogen, —N(R)₂, —OR, Cy^{C}, or an optionally substituted C₁₋₆ aliphatic group. In some embodiments, R⁶ is hydrogen.

In some embodiments of R⁶, Cy^{C} is a 3- to 7-membered saturated or partially unsaturated monocyclic carbocyclyl substituted with 0-4 L^{D}-R^{D} groups. In some embodiments of R⁶, Cy^{C} is a cyclopropyl group substituted with 0-4 L^{D}-R^{D} groups. In some embodiments, L^{D} is a covalent bond and R^{D} is selected from halogen and optionally substituted C₁₋₆ aliphatic. In some embodiments of R⁶, Cy^{C} is an unsubstituted cyclopropyl group.

In some embodiments, R⁷ is selected from hydrogen or L^{C}-R^{C}, wherein L^{C} is a covalent bond, and wherein R^{C} is Cy^{C}.

In some embodiments, R⁷ is hydrogen.

In some embodiments of R⁷, Cy^{C} is:

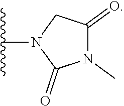 [SMILES: *N1CC(=O)N(C)C1=O]

In some embodiments, R⁸ is hydrogen.

In some embodiments, R⁸ is selected from —OR or an optionally substituted C₁₋₆ aliphatic group.

In some embodiments, a provided compound is of Formulae II-a or II-b:

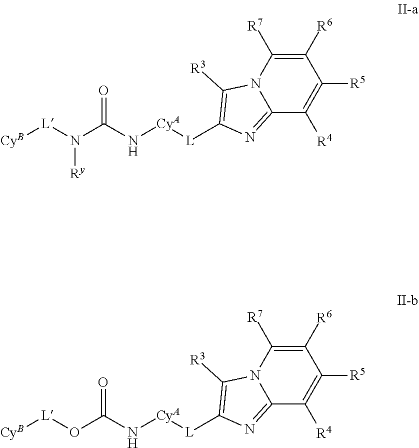

or a pharmaceutically acceptable salt thereof, wherein each of Cy^{A}, Cy^{B}, L, L′, R^{y}, R³, R⁴, R⁵, R⁶, and R⁷ is defined and described in classes and subclasses herein, both singly and in combination.

It will be understood that, unless otherwise specified or prohibited by the foregoing definition of Formulae II-a or II-b embodiments of variables Cy^{A}, Cy^{B}, L, L′, R^{y}, R³, R⁴, R⁵, R⁶, and R⁷ as defined above and described in classes and subclasses herein, also apply to compounds of Formulae II-a or II-b, both singly and in combination.

In some embodiments, a provided compound is of Formulae III-a-1, III-b-1, III-a-2, III-b-2 III-a-3, or III-b-3:

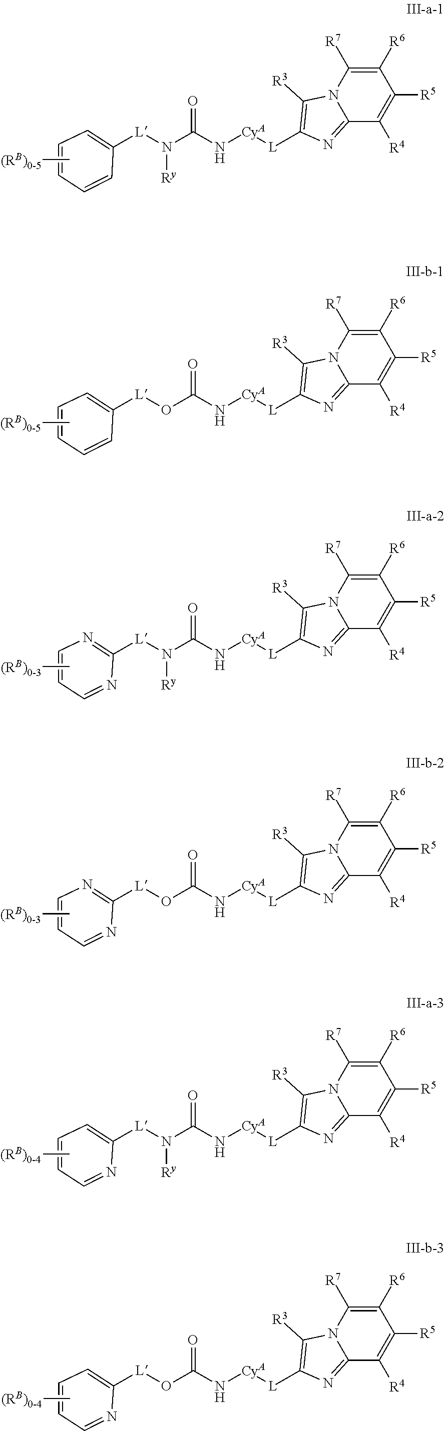

or a pharmaceutically acceptable salt thereof, wherein each of Cy^{A}, R^{B}, L, L′, R^{y}, R³, R⁴, R⁵, R⁶, and R⁷ is defined and described in classes and subclasses herein, both singly and in combination.

It will be understood that, unless otherwise specified or prohibited by the foregoing definition of Formulae III-a-1, III-b-1, III-a-2, III-b-2, III-a-3, or III-b-3 embodiments of variables Cy^{A}, Cy^{B}, L, L′, R^{y}, R³, R⁴, R⁵, R⁶, and R⁷ as defined above and described in classes and subclasses herein, also apply to compounds of Formulae III-a-1, III-b-1, III-a-2, III-b-2, III-a-3, or III-b-3, both singly and in combination.

In some embodiments, a provided compound is of Formulae IV-a-1, IV-b-1, IV-a-2, or IV-b-2:

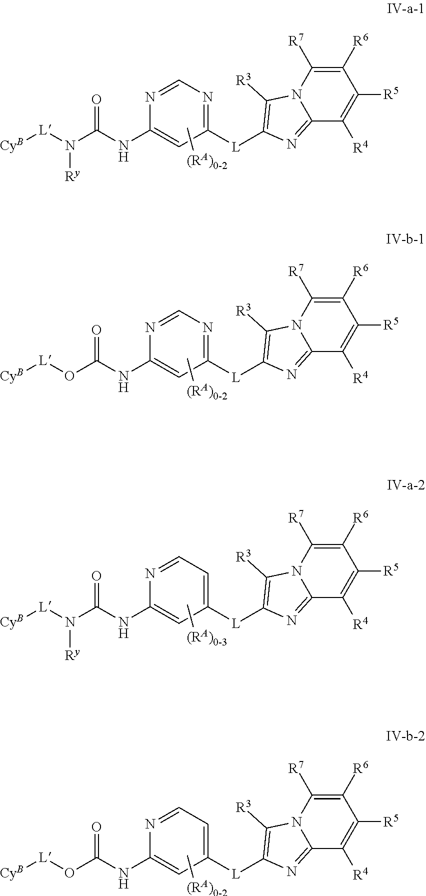

or a pharmaceutically acceptable salt thereof, wherein each of R^{A}, Cy^{B}, L, L′, R^{y}, R³, R⁴, R⁵, R⁶, and R⁷ is defined and described in classes and subclasses herein, both singly and in combination.

It will be understood that, unless otherwise specified or prohibited by the foregoing definition of Formulae IV-a-1, IV-b-1, IV-a-2, or IV-b-2 embodiments of variables R^{A}, Cy^{B}, L, L′, R^{y}, R³, R⁴, R⁵, R⁶, and R⁷ as defined above and described in classes and subclasses herein, also apply to compounds of Formulae IV-a-1, IV-b-1, IV-a-2, or IV-b-2, both singly and in combination.

In some embodiments, a provided compound is of Formula V:

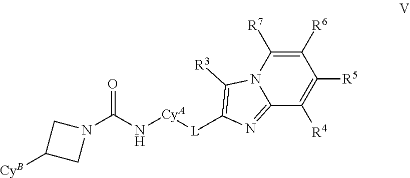

or a pharmaceutically acceptable salt thereof, wherein each of Cy^{A}, Cy^{B}, L, R³, R⁴, R⁵, R⁶, and R⁷ is defined and described in classes and subclasses herein, both singly and in combination.

It will be understood that, unless otherwise specified or prohibited by the foregoing definition of Formula V embodiments of variables Cy^{A}, Cy^{B}, L, R³, R⁴, R⁵, R⁶, and R⁷ as defined above and described in classes and subclasses herein, also apply to compounds of Formula V, both singly and in combination.

In some embodiments, a provided compound is of Formulae VI-a, VI-b, or VI-c:

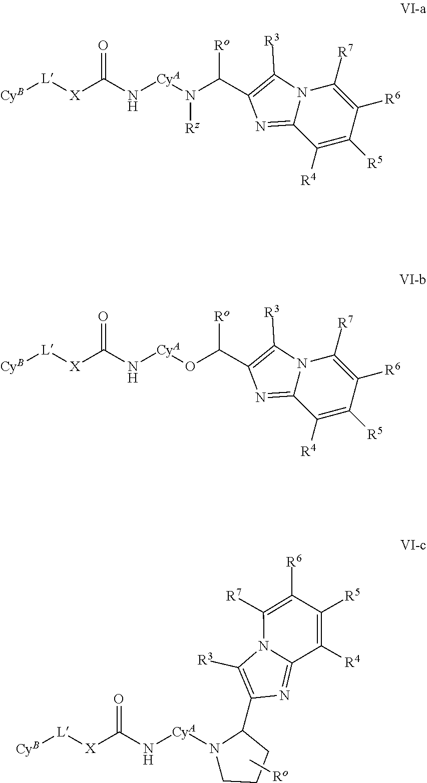

or a pharmaceutically acceptable salt thereof, wherein each of Cy^{A}, Cy^{B}, X, L′, R^{∘}, R^{z}, R³, R⁴, R⁵, R⁶, and R⁷ is defined and described in classes and subclasses herein, both singly and in combination.

It will be understood that, unless otherwise specified or prohibited by the foregoing definition of Formulae VI-a, VI-b, or VI-c embodiments of variables Cy^{A}, Cy^{B}, X, L′, R^{∘}, R³, R⁴, R⁵, R⁶, and R⁷ as defined above and described in classes and subclasses herein, also apply to compounds of Formulae VI-a, VI-b, or VI-c, both singly and in combination.

In certain embodiments, a provided compound is selected from the group consisting of: 3-Chlorobenzyl (6-(((6-cyclopropylimidazo[1,2-a]pyridin-2-yl)methyl)amino)pyrimidin-4-yl)carbamate (I-1);
(R)-1-(3-chlorophenyl)ethyl (6-(((6-cyclopropylimidazo[1,2-a]pyridin-2-yl)methyl)amino)pyrimidin-4-yl)carbamate (I-2);
(S)-1-(3-chlorophenyl)ethyl (6-(((6-cyclopropylimidazo[1,2-a]pyridin-2-yl)methyl)amino)pyrimidin-4-yl)carbamate (I-3);
3-methoxybenzyl (6-(((6-cyclopropylimidazo[1,2-a]pyridin-2-yl)methyl)amino)pyrimidin-4-yl)carbamate (I-4);
1-(3-chlorobenzyl)-3-(6-(((6-cyclopropylimidazo[1,2-a]pyridin-2-yl)methyl)amino)pyrimidin-4-yl)-1-methylurea (I-5);
1-(3-chlorophenyl)propyl (6-(((6-cyclopropylimidazo[1,2-a]pyridin-2-yl)methyl)amino)pyrimidin-4-yl)carbamate (I-6);
1-(3-chloro-2-fluorophenyl)ethyl (6-(((6-cyclopropylimidazo[1,2-a]pyridin-2-yl)methyl)amino)pyrimidin-4-yl)carbamate (I-7);
5-chloro-2-(1H-tetrazol-1-yl)benzyl (6-(((6-cyclopropylimidazo[1,2-a]pyridin-2-yl)methyl)amino)pyrimidin-4-yl)carbamate (I-8);
2-(3-chlorophenyl)-2-hydroxyethyl (6-(((6-cyclopropylimidazo[1,2-a]pyridin-2-yl)methyl)amino)pyrimidin-4-yl)carbamate (I-9);
1-(5-chloro-2-(1H-tetrazol-1-yl)benzyl)-3-(4-(((6-cyclopropylimidazo[1,2-a]pyridin-2-yl)methyl)amino)pyridin-2-yl)urea (I-10);
5-chloro-2-(1H-tetrazol-1-yl)benzyl (4-(((6-cyclopropylimidazo[1,2-a]pyridin-2-yl)methyl)amino)pyridin-2-yl)carbamate (I-11);
5-chloro-2-(1H-tetrazol-1-yl)benzyl (4-((6-cyclopropylimidazo[1,2-a]pyridin-2-yl)methoxy)pyridin-2-yl)carbamate (I-12);
3-chlorobenzyl (4-(((6-cyclopropylimidazo[1,2-a]pyridin-2-yl)methyl)amino)pyridin-2-yl)carbamate (I-13);
3-chlorobenzyl (3-((6-cyclopropylimidazo[1,2-a]pyridin-2-yl)methyl)-[1,2,4]triazolo[4,3-a]pyrazin-6-yl)carbamate (I-14);
1-(3-chlorophenyl)ethyl (4-(((6-cyclopropylimidazo[1,2-a]pyridin-2-yl)methyl)amino)pyridin-2-yl)carbamate (I-15);
3-chlorobenzyl (1-((6-cyclopropylimidazo[1,2-a]pyridin-2-yl)methyl)-1H-imidazo[4,5-c]pyridin-6-yl)carbamate (I-16);
1-(3-chlorophenyl)ethyl (1-((6-cyclopropylimidazo[1,2-a]pyridin-2-yl)methyl)-1H-imidazo[4,5-c]pyridin-6-yl)carbamate (I-17);
3-chlorobenzyl (1-((6-cyclopropylimidazo[1,2-a]pyridin-2-yl)methyl)-1H-pyrazolo[4,3-c]pyridin-4-yl)carbamate (I-18);
3-chlorobenzyl (2-((6-cyclopropylimidazo[1,2-a]pyridin-2-yl)methyl)-1H-pyrazolo[4,3-c]pyridin-4-yl)carbamate (I-19);
3-chlorobenzyl (1-((6-cyclopropylimidazo[1,2-a]pyridin-2-yl)methyl)-1H-[1,2,3]triazolo[4,5-c]pyridin-6-yl)carbamate (I-20);
1-(5-chloro-2-(1H-tetrazol-1-yl)phenyl)ethyl (4-(((6-cyclopropylimidazo[1,2-a]pyridin-2-yl)methyl)amino)pyridin-2-yl)carbamate (I-21);
5-chloro-2-(1H-tetrazol-1-yl)benzyl (2-((6-cyclopropylimidazo[1,2-a]pyridin-2-yl)methyl)-2H-pyrazolo[4,3-c]pyridin-4-yl)carbamate (I-22);
5-chloro-2-(1H-tetrazol-1-yl)benzyl (1-((6-cyclopropylimidazo[1,2-a]pyridin-2-yl)methyl)-1H-imidazo[4,5-c]pyridin-6-yl)carbamate (I-23);
1-(3-chlorobenzyl)-3-(6-(((6-cyclopropylimidazo[1,2-a]pyridin-2-yl)methyl)amino)pyrimidin-4-yl)urea (I-24);
3-(3-chlorophenyl)-N-(6-(((6-cyclopropylimidazo[1,2-a]pyridin-2-yl)methyl)amino)pyrimidin-4-yl)azetidine-1-carboxamide (I-25);
(4-chloropyridin-2-yl)methyl (6-(((6-cyclopropylimidazo[1,2-a]pyridin-2-yl)methyl)amino)pyrimidin-4-yl)carbamate (I-26);
1-((4-chloropyridin-2-yl)methyl)-3-(6-(((6-cyclopropylimidazo[1,2-a]pyridin-2-yl)methyl)amino)pyrimidin-4-yl)urea (I-27);
(4-chloropyridin-2-yl)methyl (6-((2R,4S)-2-(6-cyclopropylimidazo[1,2-a]pyridin-2-yl)-4-hydroxypyrrolidin-1-yl)pyrimidin-4-yl)carbamate (I-28);
1-((4-chloropyrimidin-2-yl)methyl)-3-(6-(((6-cyclopropylimidazo[1,2-a]pyridin-2-yl)methyl)amino)pyrimidin-4-yl)urea (I-29);
1-(6-(((6-cyclopropylimidazo[1,2-a]pyridin-2-yl)methyl)amino)pyrimidin-4-yl)-3-((4-methoxypyrimidin-2-yl)methyl)urea (I-30);
N-(6-((2R,4S)-2-(6-cyclopropylimidazo[1,2-a]pyridin-2-yl)-4-hydroxypyrrolidin-1-yl)pyrimidin-4-yl)-3-phenylazetidine-1-carboxamide (I-31);
1-(4-chlorobicyclo[4.2.0]octa-1(6),2,4-trien-7-yl)-3-(6-(((6-cyclopropylimidazo[1,2-a]pyridin-2-yl)methyl)amino)pyrimidin-4-yl)urea (I-32);
N-(6-((2R,4S)-2-(6-cyclopropylimidazo[1,2-a]pyridin-2-yl)-4-hydroxypyrrolidin-1-yl)pyrimidin-4-yl)-3-(pyrimidin-2-yl)azetidine-1-carboxamide (I-33); and
(4-methylpyrimidin-2-yl)methyl (6-(((6-cyclopropylimidazo[1,2-a]pyridin-2-yl)methyl)amino)pyrimidin-4-yl)carbamate (I-34);
or a pharmaceutically acceptable salt thereof.

Compounds explicitly disclosed herein may be claimed as an individual compound, including where there is no reference to stereochemistry.

### C. Pharmaceutical Compositions

In another aspect, the present invention provides pharmaceutical compositions comprising a compound of the present disclosure, including Formulae (I)-(VI-c), or a compound of Formulae (I)-(VI-c) or an exemplified compound in combination with a pharmaceutically acceptable excipient (e.g., carrier).

The pharmaceutical compositions include optical isomers, diastereomers, or pharmaceutically acceptable salts of the inhibitors disclosed herein. A compound of Formulae (I)-(VI-c) included in the pharmaceutical composition may be covalently attached to a carrier moiety, as described above. Alternatively, a compound of Formulae (I)-(VI-c) included in the pharmaceutical composition is not covalently linked to a carrier moiety.

A “pharmaceutically acceptable carrier,” as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

The compounds of the invention can be administered alone or can be coadministered to the subject. Coadministration is meant to include simultaneous or sequential administration of the compounds individually or in combination (more than one compound). The preparations can also be combined, when desired, with other active substances (e.g. to reduce metabolic degradation).

In some embodiments, a test agent as described herein can be incorporated into a pharmaceutical composition for administration by methods known to those skilled in the art and described herein for provided compounds.

### D. Formulations

Compounds of the present invention can be prepared and administered in a wide variety of oral, parenteral, and topical dosage forms. Thus, the compounds of the present invention can be administered by injection (e.g. intravenously, intramuscularly, intracutaneously, subcutaneously, intraduodenally, or intraperitoneally). In some embodiments compounds of the present disclosure are administered orally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds of the invention can also be incorporated into liposomes or administered via transdermal pumps or patches. Pharmaceutical admixtures suitable for use in the present invention include those described, for example, in Pharmaceutical Sciences (17th Ed., Mack Pub. Co., Easton, PA) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

### E. Effective Dosages

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat HAE, such compositions will contain an amount of active ingredient effective to achieve the desired result (e.g. inhibiting PKa and/or decreasing the amount of bradykinin in a subject).

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the disease responsive to PKa inhibition); presence of other diseases or other health-related problems; kind of concurrent treatment; and complications from any disease or treatment regimen. Other therapeutic regimens or agents can be used in conjunction with the methods and compounds of the invention.

For any provided compound or test agent, the therapeutically effective amount can be initially determined from cell culture assays. Target concentrations will be those concentrations of active compound(s) that are capable of decreasing PKa enzymatic activity as measured, for example, using the methods described.

Therapeutically effective amounts for use in humans may be determined from animal models. For example, a dose for humans can be formulated to achieve a concentration that has been found to be effective in animals. The dosage in humans can be adjusted by monitoring PKa inhibition and adjusting the dosage upwards or downwards, as described above.

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects. In some embodiments a compound of the disclosure or a pharmaceutical composition comprising the same is provided as a unit dose.

In one aspect, compounds provided herein display one or more improved pharmacokinetic (PK) properties (e.g., Cₘₐₓ, tₘₐₓ, Cₘᵢₙ, t_{1/2}, AUC, CL, bioavailability, etc.) when compared to a reference compound. In some embodiments, a reference compound is a PKa inhibitor known in the art. In some embodiments, a reference compound is a PKa inhibitor selected from those disclosed in PCT Publication Number WO 2019/178129.

### F. Methods of Treatment

The present disclosure provides compounds and pharmaceutical compositions comprising the same for use in medicine, i.e., for use in treatment. The present disclosure further provides the use of any compounds described herein for inhibiting the activity of PKa, which would be beneficial to treatment of PKa-mediated diseases and conditions. Exemplary PKa-mediated disorders include edema, which refers to swelling in the whole body of a subject or a part thereof due to inflammation or injury when small blood vessels become leaky and releases fluid into nearby tissues. In some examples, the edema is HAE. In other examples, the edema occurs in eyes, e.g., diabetic macular edema (DME). The present disclosure provides methods of inhibiting the activity of PKa. In certain embodiments, the application provides a method of inhibiting the activity of PKa in vitro via contacting any of the compounds described herein with PKa molecules in a sample, such as a biological sample. In certain embodiments, the application provides a method of inhibiting the activity of PKa in vivo via delivering an effective amount of any of the compounds described herein to a subject in need of the treatment through a suitable route.

In certain embodiments, the methods comprise administering to a subject in need thereof (e.g., a subject such as a human patient, for example with edema) any of the compounds described herein or a pharmaceutically acceptable salt thereof. In certain embodiments, the methods comprise administering a compound of Formulae (I)-(VI-c), or a pharmaceutically acceptable salt or composition thereof, to a subject in need thereof. In some embodiments, the method comprises administering a pharmaceutical composition comprising a compound of Formulae (I)-(VI-c), or a pharmaceutically acceptable salt to a subject in need thereof.

In certain embodiments, the subject to be treated by any of the methods described herein is a human patient having, suspected of having, or at risk for edema, for example, HAE or diabetic macular edema (DME). A subject having an edema can be identified by routine medical examination, e.g., laboratory tests. A subject suspected of having an edema might show one or more symptoms of the disease/disorder. A subject at risk for edema can be a subject having one or more of the risk factors associated with the disease, for example, deficiency in C1-INH as for HAE.

In certain embodiments, provided herein are methods of alleviating one or more symptoms of HAE in a human patient who is suffering from an HAE attack. Such a patient can be identified by routine medical procedures. An effective amount of one or more of the provided compounds can be given to the human patient via a suitable route, for example, those described herein. The compounds described herein may be used alone, or may be used in combination with other anti-HAE agents, for example, a C1 esterase inhibitor (e.g., Cinryze® or Berinert®), a PKa inhibitor (e.g., ecallantide or lanadelumab) or a bradykinin B2 receptor antagonist (e.g., Firazyr®).

In other embodiments, provided herein are methods or reducing the risk of HAE attack in a human HAE patient who is in quiescent stage. Such a patient can be identified based on various factors, including history of HAE attack. An effective amount of one or more of the compounds can be given to the human patient via a suitable route, for example, those described herein. The compounds described herein may be used alone, or may be used in combination with other anti-HAE agents, for example, a C1 esterase inhibitor (e.g., Cinryze® or Berinert®), a PKa inhibitor (e.g., ecallantide or lanadelumab) or a bradykinin B2 receptor antagonist (e.g., Firazyr®).

In some embodiments, provided herein is prophylactic treatment of HAE in human patients having risk to HAE attacks with one or more of the compounds described herein. In some embodiments, patients suitable for prophylactic treatment of HAE are human subjects suffering from HAE (e.g., having history of HAE attacks). In some embodiments, patients suitable for such prophylactic treatment are human subjects where a physician determines a history of HAE attacks warrants a prophylactic approach (e.g., human subjects experiencing more than a particular average number of attacks over a time period, including by way of nonlimiting example, one, two, or more attacks per month). Alternatively, patients suitable for the prophylactic treatment may be human subjects having no HAE attack history but bearing one or more risk factors for HAE (e.g., family history, genetic defects in C1-INH gene, etc.) Such prophylactic treatment may involve the compounds described herein as the sole active agent, or involve additional anti-HAE agents, such as those described herein.

In certain embodiments, provided herein are methods for preventing or reducing edema in an eye of a subject (e.g., a human patient). In some examples, the human patient is a diabetic having, suspected of having, or at risk for diabetic macular edema (DME). DME is the proliferative form of diabetic retinopathy characterized by swelling of the retinal layers, neovascularization, vascular leak, and retinal thickening in diabetes mellitus due to leaking of fluid from blood vessels within the macula. To practice this method, an effective amount of one or more of the compounds described herein, or pharmaceutically acceptable salts thereof, may be delivered into the eye of the subject where treatment is needed. For example, the compound may be delivered topically, by intraocular injection, or intravitreal injection. A subject may be treated with the compound as described herein, either as the sole active agent, or in combination with another treatment for DME. Non-limiting examples of treatment for DME include laser photocoagulation, steroids, VEGF pathway targeting agents (e.g., Lucentis® (ranibizumab) or Eylea® (aflibercept)), and/or anti-PDGF agents.

In certain embodiments, the methods disclosed herein comprise administering to the subject an effective amount of a compound of Formulae (I)-(VI-c), or a pharmaceutically acceptable salt or composition thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as the compound of Formulae (I)-(VI-c), or at different times than the compound of Formulae (I)-(VI-c). For example, the compound of Formulae (I)-(VI-c) and any additional pharmaceutical agent(s) may be on the same dosing schedule or different dosing schedules. All or some doses of the compound of Formulae (I)-(VI-c) may be administered before all or some doses of an additional pharmaceutical agent, after all or some does an additional pharmaceutical agent, within a dosing schedule of an additional pharmaceutical agent, or a combination thereof. The timing of administration of the compound of Formulae (I)-(VI-c) and additional pharmaceutical agents may be different for different additional pharmaceutical agents.

In certain embodiments, the additional pharmaceutical agent comprises an agent useful in the treatment of an edema, such as HAE or DME. Examples of such agents are provided herein.

Also provided is used of a compound of the present disclosure for the manufacture of a medicament for a condition/disease disclosed herein.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

The background section of this specification contains relevant technical information and may be used as basis for amendment. Subject headings herein are employed to divide the document into sections and are not intended to be used to construe the meaning of the disclosure provided herein.

The present specification claims priority from U.S. Provisional Application No. 63/162,494, filed Mar. 17, 2021, and incorporated herein by reference. This application may be used as basis for corrections to the present specification, especially in respect of chemical structures disclosed therein.

### IV. EXAMPLES

### Synthesis of Intermediates

### Synthesis of 1-(6-cyclopropylimidazo[1,2-a]pyridin-2-yl)-2,2,2-trifluoroethan-1-one

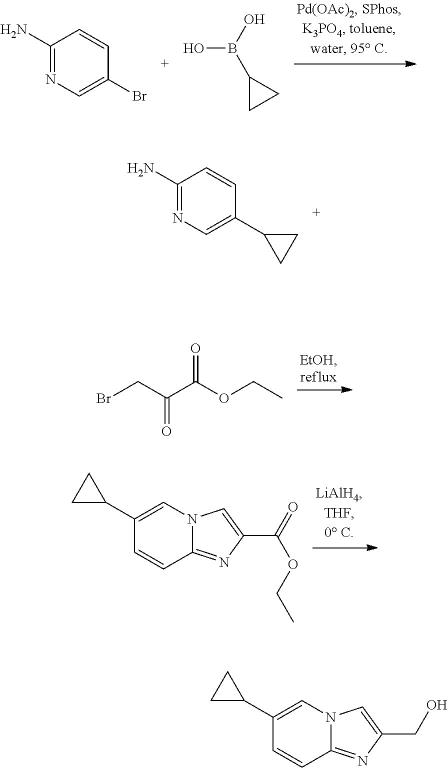

Synthesis of 5-cyclopropylpyridin-2-amine. 5-Bromopyridin-2-amine (10 g, 58 mmol), cyclopropylboronic acid (12 g, 120 mmol), SPhos (2.4 g, 5.8 mmol) and K₃PO₄ (43 g, 200 mmol) were placed under N₂ atmosphere and suspended in toluene (220 mL) and water (22 mL). The resulting suspension was degassed for 10 min and Pd(OAc)₂ (0.65 g, 2.9 mmol) was added. The reaction mixture was stirred at 95° C. for 16 h under N₂ atmosphere. The mixture was cooled to room temperature, diluted with water (30 mL), and extracted with ethyl acetate (3×30 mL). The organic phase was washed with brine (150 mL), passed through a hydrophobic frit, and concentrated in vacuo. The residue was purified by silica gel chromatography, eluting with a gradient of 10-95% EtOAc in hexane to give the title compound (5.3 g, 68%) as a red solid. ESI-MS (M+H)+: 135.0, ¹H NMR (400 MHz, DMSO) δ 7.78 (d, J=2.5 Hz, 1H), 7.07 (dd, J=2.5, 8.6 Hz, 1H), 6.39 (d, J=8.4 Hz, 1H), 5.66 (s, 2H), 1.80-1.72 (m, 1H), 0.86-0.81 (m, 2H), 0.56-0.52 (m, 2H).

Synthesis of ethyl 6-cyclopropylimidazo[1,2-a]pyridine-2-carboxylate. A solution of 5-cyclopropylpyridin-2-amine (5.3 g, 40 mmol) and ethyl 3-bromo-2-oxopropanoate (5.9 mL, 47 mmol) in ethanol (110 mL) was stirred at reflux for 1 hour. The mixture was cooled to room temperature and concentrated in vacuo. The residue was dissolved in DCM (100 mL), washed with Na₂CO₃ (sat. aq., 60 mL), passed through a hydrophobic frit, and concentrated in vacuo. The residue was purified by silica gel chromatography, eluting with a gradient of 20-80% EtOAc in hexane to give the title compound (5 g, 54%) as an orange solid. ESI-MS (M+H)+: 231.2, ¹H NMR (400 MHz, CDCl₃) δ 8.10-8.09 (m, 1H), 7.91-7.89 (m, 1H), 7.57 (d, J=9.5 Hz, 1H), 6.99 (dd, J=1.6, 9.5 Hz, 1H), 4.45 (q, J=7.1 Hz, 2H), 1.94-1.86 (m, 1H), 1.44 (t, J=7.2 Hz, 3H), 1.03-0.97 (m, 2H), 0.73-0.68 (m, 2H).

Synthesis of (6-cyclopropylimidazo[1,2-a]pyridin-2-yl)methanol. LiAlH₄ (1 M in THF, 42.99 mmol, 42.99 mL) was added to a solution of ethyl 6-cyclopropylimidazo[1,2-a]pyridine-2-carboxylate (21.50 mmol, 4.95 g) in THF (25 mL) cooled to 0° C., under N₂. The mixture was stirred for 1 hour, then EtOAc (5 mL) was added dropwise. The mixture was stirred at room temperature for 1 hour, then washed with water (30 mL) and brine (30 mL). The organics were dried over MgSO₄ then concentrated in vacuo to give the title compound which was used without further purification. ¹H NMR (400 MHz, CDCl₃) δ 7.86 (s, 1H), 7.46-7.42 (m, 2H), 6.92 (dd, J=1.8, 9.4 Hz, 1H), 4.82 (s, 2H), 3.88-3.84 (m, 1H), 1.87 (s, 1H), 0.99-0.93 (m, 2H), 0.69-0.64 (m, 2H).

### Synthesis of (6-cyclopropylimidazo[1,2-a]pyridin-2-yl)methanamine

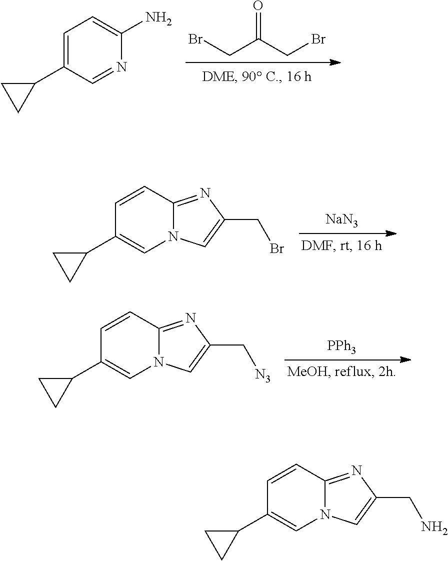

### Synthesis of 2-(bromomethyl)-6-cyclopropylimidazo[1,2-a]pyridine

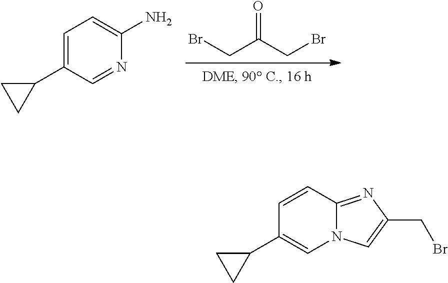 [SMILES: Nc1ccc(C2CC2)cn1 ; O=C(CBr)CBr ; BrCc1cn2cc(C3CC3)ccc2n1]

A mixture of 5-cyclopropylpyridin-2-amine (670 mg, 5.0 mmol) and 1,3-dibromopropan-2-one (1.61 g, 7.5 mmol) in DME (20.0 mL) was stirred at 90° C. under N₂ for 16 h. The reaction mixture was cooled to room temperature, quenched with sat. NaHCO₃ solution (50 mL), and extracted with EtOAc (3×50 ml). The combined organic layers were washed with brine (50 mL), dried over Na₂SO₄, and concentrated in vacuo. The crude residue was purified by column chromatography (eluent: DCM/MeOH=50/1˜30/1) to give the product 2-(bromomethyl)-6-cyclopropylimidazo[1,2-a]pyridine (880 mg, 70%) as a yellow solid. ESI-MS [M+H]⁺: 252.2.

### Synthesis of 2-(azidomethyl)-6-cyclopropylimidazo[1,2-a]pyridine

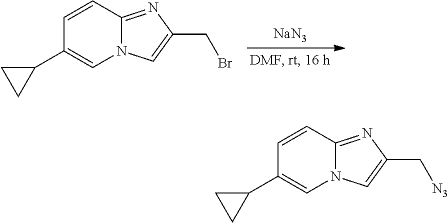 [SMILES: BrCc1cn2cc(C3CC3)ccc2n1 ; [N-]=[N+]=NCc1cn2cc(C3CC3)ccc2n1 ; "NaN3"]

A mixture of 2-(bromomethyl)-6-cyclopropylimidazo[1,2-a]pyridine (753 mg, 3 mmol) and NaN₃ (244 mg, 3.75 mmol) in DMF (10.0 mL) was stirred at room temperature under N₂ for 16 h. The mixture was diluted with EtOAc (100 mL) and washed with brine (3×50 mL). The organic layers were dried over Na₂SO₄ and concentrated in vacuo to give 2-(azidomethyl)-6-cyclopropylimidazo[1,2-a]pyridine (730 mg, crude) as a yellow solid, which was used for the next step without purification. ESI-MS [M+H]⁺: 214.2.

### Synthesis of (6-cyclopropylimidazo[1,2-a]pyridin-2-yl)methanamine

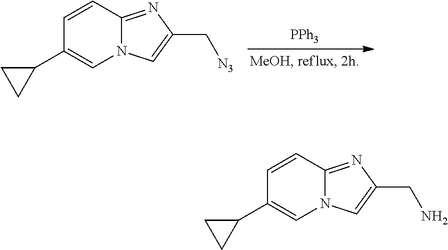 [SMILES: [N-]=[N+]=NCc1cn2cc(C3CC3)ccc2n1 ; NCc1cn2cc(C3CC3)ccc2n1 ; "PPh3"]

A mixture of 2-(azidomethyl)-6-cyclopropylimidazo[1,2-a]pyridine (730 mg, crude) and PPh₃ (983 mg, 3.75 mmol) in MeOH (25 mL) was stirred at reflux for 2 h. The mixture was concentrated in vacuo and purified by Prep-TLC (eluent: DCM/MeOH=10/1) to give (6-cyclopropylimidazo[1,2-a]pyridin-2-yl)methanamine (450 mg, 81% over 2 steps) as a yellow oil. ESI-MS [M+H]+: 188.2.

### Synthesis of 2-((2R,4S)-4-((tert-butyldimethylsilyl)oxy)pyrrolidin-2-yl)-6-cyclopropylimidazo[1,2-a]pyridine

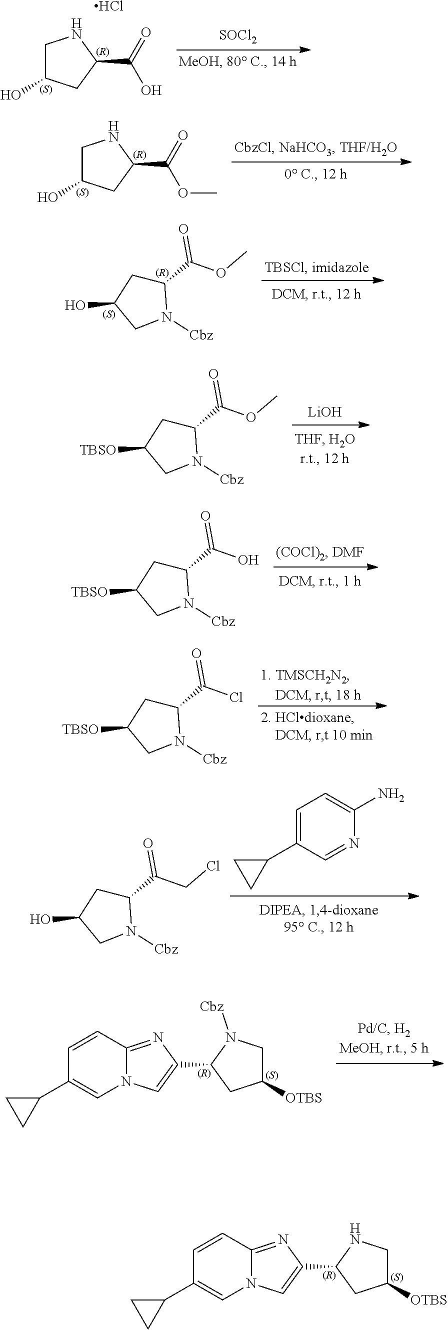

### Synthesis of methyl (2R,4S)-4-hydroxypyrrolidine-2-carboxylate

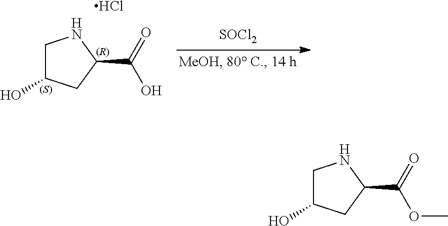

To a solution of (2R, 4S)-4-hydroxypyrrolidine-2-carboxylic acid hydrochloride (5 g, 29.8 mmol) in MeOH (50 mL) was added SOCl₂ (10.6 g, 89.4 mmol) slowly at 0° C. After stirring at 80° C. for 12 h, the reaction was concentrated in vacuo to give methyl (2R, 4S)-4-hydroxypyrrolidine-2-carboxylate (4 g, crude) as a grey solid. ESI-MS [M+H]+: 146.2.

### Synthesis of 1-benzyl 2-methyl (2R,4S)-4-hydroxypyrrolidine-1,2-dicarboxylate

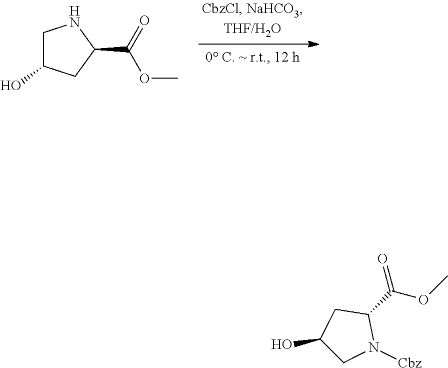

To a solution of methyl (2R, 4S)-4-hydroxypyrrolidine-2-carboxylate (4 g, 27.5 mmol) in THF (80 mL) was added saturated aqueous NaHCO₃ (60 ml), followed by CbzCl (5.6 g, 33.1 mmol) at 0° C. The reaction mixture was stirred at 0° C. for another 1 h and then extracted with EtOAc (3×50 mL). The combined organic layers were washed with brine, dried over Na₂SO₄, and concentrated in vacuo to give the crude, which was purified by silica gel chromatography (EtOAc/PE=1/5) to give 1-benzyl 2-methyl (2R, 4S)-4-hydroxypyrrolidine-1,2-dicarboxylate (5.8 g, 75%) as yellow oil. ESI-MS [M+H]⁺: 280.1.

### Synthesis of 1-benzyl 2-methyl (2R,4S)-4-((tert-butyldimethylsilyl)oxy)pyrrolidine-1,2-dicarboxylate

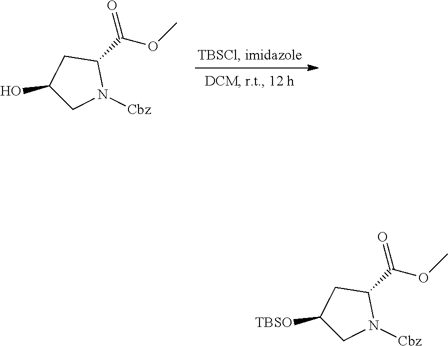 [SMILES: COC(=O)[C@H]1C[C@H](O)CN1C(=O)OCc1ccccc1 ; COC(=O)[C@H]1C[C@H](O[Si](C)(C)C(C)(C)C)CN1C(=O)OCc1ccccc1]

A mixture of 1-benzyl 2-methyl (2R,4S)-4-hydroxypyrrolidine-1,2-dicarboxylate (4 g, 14.3 mmol), TMSCl (2.3 g, 21.5 mmol), and imidazole(1.9 g, 28.6 mmol) in DCM (40 mL) was stirred at room temperature for 12 h. The reaction was washed with water (50 mL) then extracted with EtOAc (3×50 mL). The combined organic layers were washed with brine, dried over Na₂SO₄, filtered and concentrated in vacuo to give the crude, which was purified by silica gel chromatography (EtOAc/PE=1/3) to give 1-benzyl 2-methyl (2R, 4S)-4-((tert-butyldimethylsilyl)oxy)pyrrolidine-1,2-dicarboxylate (4 g, 71%) as yellow oil. ESI-MS [M+H]⁺: 394.1.

### Synthesis of (2R,4S)-1-((benzyloxy)carbonyl)-4-((tert-butyldimethylsilyl)oxy)pyrrolidine-2-carboxylic acid

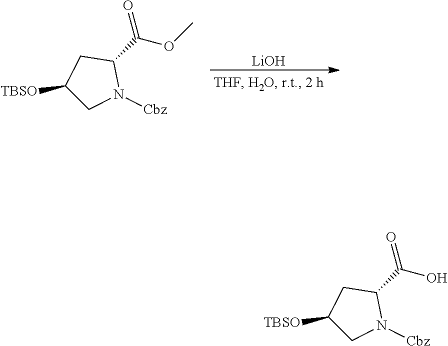 [SMILES: COC(=O)[C@H]1C[C@H](O[Si](C)(C)C(C)(C)C)CN1C(=O)OCc1ccccc1 ; "LiOH" ; CC(C)(C)[Si](C)(C)O[C@H]1C[C@H](C(=O)O)N(C(=O)OCc2ccccc2)C1]

To a solution of 1-benzyl 2-methyl (2R,4S)-4-((tert-butyldimethylsilyl)oxy)pyrrolidine-1,2-dicarboxylate (4 g, 10.2 mmol) in THF (40 mL) was added LiOH (1.25 g, 30.6 mmol) and water (4 mL). The reaction mixture was stirred at room temperature for 2 h and then concentrated in vacuo to remove THF. The pH of the resulting residue was adjusted to 3-4 with HCl (1.0 M) then the mixture was extracted with EtOAc (3×50 mL). The combined organic layers were washed with brine, dried over Na₂SO₄, filtered, and concentrated in vacuo to give the crude (2R, 4S)-1-((benzyloxy)carbonyl)-4-((tert-butyldimethylsilyl)oxy)pyrrolidine-2-carboxylic acid (3.3 g, 87%) as yellow oil. ESI-MS [M+H]⁺: 394.1.

### Synthesis of benzyl (2R,4S)-4-((tert-butyldimethylsilyl)oxy)-2-(chlorocarbonyl)pyrrolidine-1-carboxylate

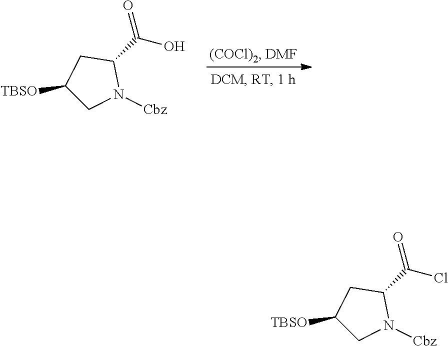

To a solution of (2R,4S)-1-((benzyloxy)carbonyl)-4-((tert-butyldimethylsilyl)oxy)pyrrolidine-2-carboxylic acid (2 g, crude from previous step) in DCM (20 mL) was added DMF (2 drops) and (COCl)₂ (1.0 g, 7.91 mmol) at 0° C. After stirring at room temperature for 1 h, the solution was concentrated in vacuo to give benzyl (2R, 4S)-4-((tert-butyldimethylsilyl)oxy)-2-(chlorocarbonyl)pyrrolidine-1-carboxylate (2.1 g crude) as yellow oil, which was used in the next step without further purification. ESI-MS [M+H]⁺: 394.1.

### Synthesis of benzyl (2R,4S)-2-(2-chloroacetyl)-4-hydroxypyrrolidine-1-carboxylate

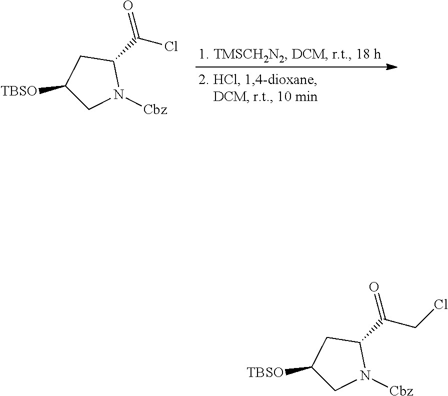 [SMILES: CC(C)(C)[Si](C)(C)O[C@H]1C[C@H](C(=O)Cl)N(C(=O)OCc2ccccc2)C1 ; "1. TMSCH2N2, DCM, r.t., 18 h" ; CC(C)(C)[Si](C)(C)O[C@H]1C[C@H](C(=O)CCl)N(C(=O)OCc2ccccc2)C1]

To a solution of benzyl (2R, 4S)-4-((tert-butyldimethylsilyl)oxy)-2-(chlorocarbonyl)pyrrolidine-1-carboxylate (400 mg, 1 mmol) in DCM (20 mL) was added TMSCH₂N₂ (1 mL, 2.0 M in hexane). After stirring at room temperature for 12 h, the solution was concentrated in vacuo to give the crude, which was re-dissolved in DCM (10 mL), and then HCl (4.0 M in 1,4-dioxane, 0.5 mL) was added. After stirring at room temperature for another 10 min, the solution was concentrated in vacuo to give the crude, which was purified by silica gel chromatography (eluent: EtOAc/PE=1/5) to give benzyl (2R, 4S)-2-(2-chloroacetyl)-4-hydroxypyrrolidine-1-carboxylate (150 mg, 36%) as yellow oil. ESI-MS [M+H]⁺: 298.2.

### Synthesis of benzyl (2R, 4S)-4-((tert-butyldimethylsilyl)oxy)-2-(6-cyclopropylimidazo[1,2-a]pyridin-2-yl)pyrrolidine-1-carboxylate

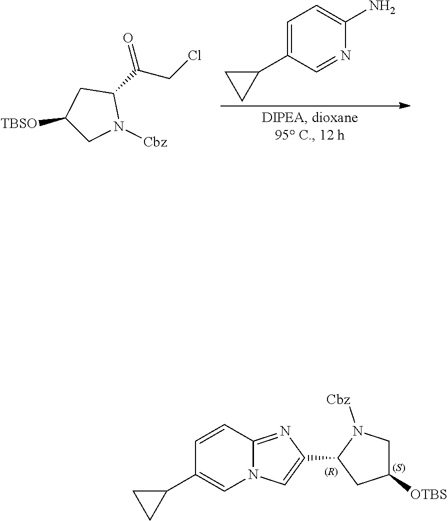

A solution of benzyl (2R,4S)-2-(2-chloroacetyl)-4-hydroxypyrrolidine-1-carboxylate (150 mg, 0.51 mmol), 5-cyclopropylpyridin-2-amine (134 mg, 1.0 mmol), and DIPEA (329 mg, 2.55 mmol) in 1.4-dioxane (10 mL) was stirred at 95° C. for 12 h. The resulting mixture was concentrated in vacuo to give the crude, which was purified with silica gel chromatography (eluent: DCM/MeOH=15/1) to give benzyl (2R,4S)-4-((tert-butyldimethylsilyl)oxy)-2-(6-cyclopropylimidazo[1,2-a]pyridin-2-yl)pyrrolidine-1-carboxylate (150 mg, 58%) as a yellow solid. ESI-MS [M+H]+: 492.1.

### Synthesis of 2-((2R,4S)-4-((tert-butyldimethylsilyl)oxy)pyrrolidin-2-yl)-6-cyclopropylimidazo[1,2-a]pyridine

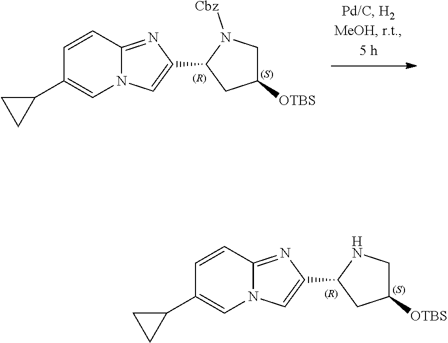

A mixture of benzyl (2R,4S)-4-((tert-butyldimethylsilyl)oxy)-2-(6-cyclopropylimidazo[1,2-a]pyridin-2-yl)pyrrolidine-1-carboxylate (150 mg, 0.31 mmol) and Pd/C (20 mg) in MeOH (10 mL) was stirred at room temperature for 5 h under a H₂ atmosphere. The reaction was filtered, then washed with MeOH (30 mL). The filtrate was concentrated in vacuo to give 2-((2R, 4S)-4-((tert-butyldimethylsilyl)oxy)pyrrolidin-2-yl)-6-cyclopropylimidazo[1,2-a]pyridine (110 mg) as a yellow solid, which was used without further purification. ESI-MS [M+H]⁺: 358.2.

### Example 1

### Synthesis of 3-Chlorobenzyl (6-(((6-cyclopropylimidazo[1,2-a]pyridin-2-yl)methyl)amino)pyrimidin-4-yl)carbamate (I-1)

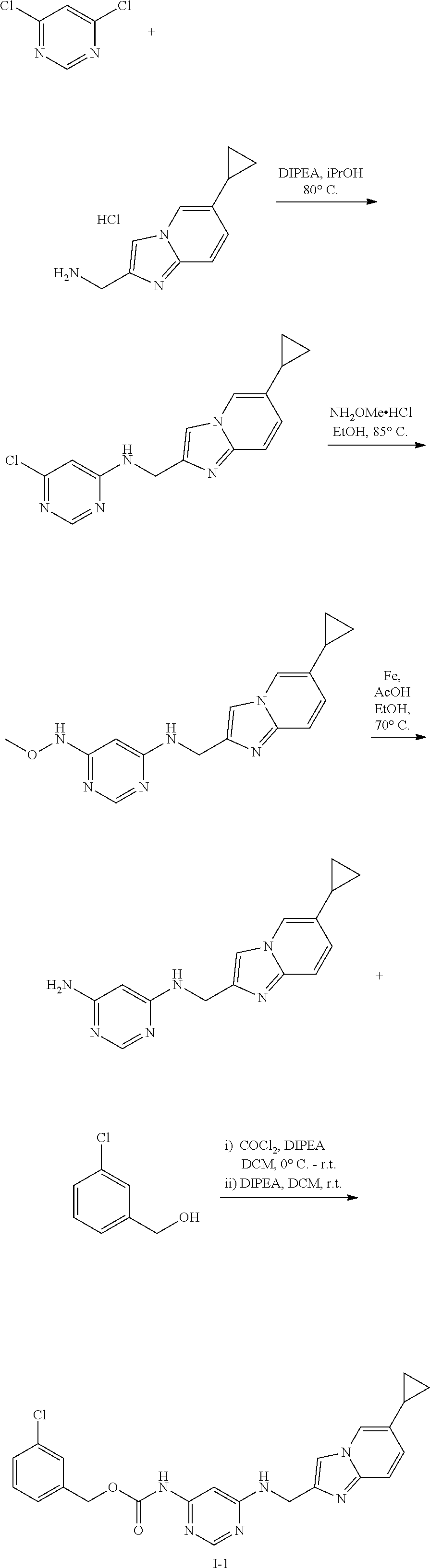

Synthesis of 6-Chloro-N-((6-cyclopropylimidazo[1,2-a]pyridin-2-yl)methyl)pyrimidin-4-amine. A solution of 4,6-dichloropyrimidine (302 mg, 2.6 mmol), (6-cyclopropylimidazo[1,2-a]pyridin-2-yl)methanamine hydrochloride (400 mg, 2.1 mmol) and DIPEA (0.93 mL, 5.3 mmol) in iPrOH (20 mL) was heated at 80° C. for 2 h. The reaction mixture was concentrated in vacuo and the residue was triturated with Et₂O. The crude solid was re-dissolved in DCM (35 mL) and washed with water. The combined organics were passed through a hydrophobic frit, then concentrated in vacuo to give the title compound (560 mg, 87%) as a colourless oil. ¹H NMR (400 MHz, DMSO) δ 8.33-8.29 (m, 2H), 8.21-8.20 (m, 1H), 7.68 (s, 1H), 7.39 (d, J=9.3 Hz, 1H), 6.98 (dd, J=1.5, 9.3 Hz, 1H), 6.63 (s, 1H), 4.61 (s, 2H), 1.95-1.88 (m, 1H), 0.94-0.89 (m, 2H), 0.69-0.64 (m, 2H).

Synthesis of N-((6-Cyclopropylimidazo[1,2-a]pyridin-2-yl)methyl)-6-(methoxyamino)pyrimidin-4-amine. A solution of 6-chloro-N-((6-cyclopropylimidazo[1,2-a]pyridin-2-yl)methyl)pyrimidin-4-amine (1.6 g, 5.5 mmol) and O-methyl-hydroxylamine hydrochloride (6.9 g, 82 mmol) in ethanol (40 mL) was heated at 85° C. for 24 h. The reaction mixture was diluted with NaHCO₃ (sat. aq.) and extracted with EtOAc. The combined organics were dried over MgSO₄, filtered, and concentrated in vacuo to give the title compound (1.52 g, 90%) as a brown gum, which was used without further purification. ESI-MS (M+H)+: 311, ¹H NMR (400 MHz, DMSO) δ 9.56 (br s, 1H), 8.30 (s, 1H), 8.00 (s, 1H), 7.62 (s, 1H), 7.49-7.40 (m, 1H), 7.37 (d, J=9.3 Hz, 1H), 6.95 (dd, J=1.4, 9.2 Hz, 1H), 5.84 (s, 1H), 4.50 (s, 2H), 3.32 (s, 3H), 1.96-1.85 (m, 1H), 0.95-0.85 (m, 2H), 0.70-0.60 (m, 2H).

Synthesis of N⁴-((6-Cyclopropylimidazo[1,2-a]pyridin-2-yl)methyl)pyrimidine-4,6-diamine. A solution of N-((6-cyclopropylimidazo[1,2-a]pyridin-2-yl)methyl)-6-(methoxyamino)pyrimidin-4-amine (1.5 g, 4.9 mmol) and iron powder (1.4 g, 24 mmol) in acetic acid (13 mL) and ethanol (80 mL) was heated at 70° C. for 2 h. The reaction mixture was cooled and filtered through a pad of Celite®, eluting with EtOAc. The filtrate was concentrated and loaded onto an SCX column, washed with MeOH in DCM (1:9). The product was eluted with (7 N NH₃ in MeOH) in DCM (1:9) and concentrated in vacuo to give the title compound (1.2 g, 84%) as a brown solid. ESI-MS (M+H)+: 281. ¹H NMR (400 MHz, DMSO) δ 8.30-8.29 (m, 1H), 7.87 (s, 1H), 7.57 (s, 1H), 7.36 (d, J=9.3 Hz, 1H), 6.99 (t, J=5.8 Hz, 1H), 6.95 (dd, J=1.8, 9.3 Hz, 1H), 6.02 (s, 2H), 5.41 (s, 1H), 4.43 (d, J=5.5 Hz, 2H), 1.94-1.86 (m, 1H), 0.93-0.87 (m, 2H), 0.68-0.62 (m, 2H).

Synthesis of 3-Chlorobenzyl (6-(((6-cyclopropylimidazo[1,2-a]pyridin-2-yl)methyl)amino)pyrimidin-4-yl)carbamate. Phosgene (20% in toluene, 0.42 mL, 0.80 mmol) was added dropwise to a stirred solution of 3-chlorobenzyl alcohol (0.12 mL, 1.0 mmol) and DIPEA (0.44 mL, 2.5 mmol) in DCM (14 mL) at 0° C. The mixture was stirred at 0° C. for 30 min then warmed to room temperature and stirred for 1 h. The resulting solution was added over 1 h to a solution of N⁴-((6-cyclopropylimidazo[1,2-a]pyridin-2-yl)methyl)pyrimidine-4,6-diamine (140 mg, 1.0 mmol) and DIPEA (0.35 mL, 2.0 mmol) in MeCN (5.0 mL). The mixture was stirred at room temperature for 18 h, then K₂CO₃ (10% aq.) was added and the phases were separated. The combined organics were dried over MgSO₄, then concentrated in vacuo. The residue was purified by silica gel chromatography, eluting with a gradient of 1-10% MeOH in DCM followed by preparative HPLC to give the title compound (1.1 mg, 1%). ESI-MS (M+H)+: 449.2, ¹H NMR (400 MHz, DMSO) δ 10.18 (s, 1H), 8.29 (s, 1H), 8.16 (s, 1H), 7.82 (br s, 1H), 7.61 (s, 1H), 7.51 (s, 1H), 7.43-7.33 (m, 4H), 7.03-6.93 (m, 2H), 5.16 (s, 2H), 4.56 (br s, 2H), 1.94-1.86 (m, 1H), 0.94-0.88 (m, 2H), 0.68-0.62 (m, 2H).

### Example 2

### Synthesis of (R)-1-(3-chlorophenyl)ethyl (6-(((6-cyclopropylimidazo[1,2-a]pyridin-2-yl)methyl)amino)pyrimidin-4-yl)carbamate (I-2)

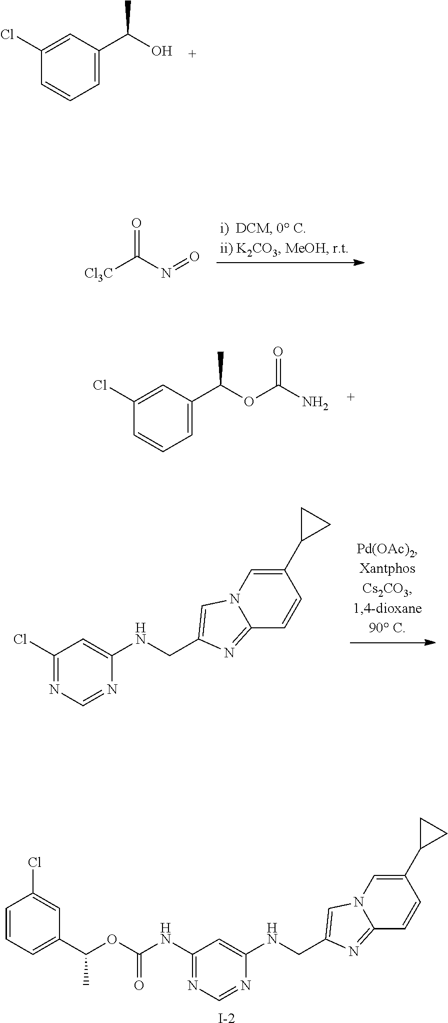

Synthesis of (R)-1-(3-chlorophenyl)ethyl carbamate. Trichloroacetyl isocyanate (46 μL, 0.38 mmol) was added dropwise to a stirred solution of (R)-1-(3-chlorophenyl)ethan-1-ol (50 mg, 0.32 mmol) in DCM (3 mL) at 0° C. After stirring with cooling for 80 min, the reaction mixture was concentrated in vacuo. The residue was re-dissolved in MeOH (3.0 mL), and K₂CO₃ (8.8 mg, 0.064 mmol) was added. The reaction mixture was stirred at room temperature for 2 d, then diluted with DCM and water. The organic layer was separated, dried over MgSO₄ and concentrated in vacuo. The residue was purified by silica gel column chromatography, eluting with a gradient of 0-50% EtOAc in cyclohexane to give the title compound (36 mg, 57%) as a colourless oil, which was used directly in the next step.

Synthesis of (R)-1-(3-chlorophenyl)ethyl (6-(((6-cyclopropylimidazo[1,2-a]pyridin-2-yl)methyl)amino)pyrimidin-4-yl)carbamate. A mixture of (R)-1-(3-chlorophenyl)ethyl carbamate (18 mg, 0.090 mmol), 6-chloro-N-((6-cyclopropylimidazo[1,2-a]pyridin-2-yl)methyl)pyrimidin-4-amine (27 mg, 0.090 mmol), tris(dibenzylideneaceetone)dipalladium(0) (17 mg, 0.018 mmol), Xantphos (21 mg, 0.036 mmol) and Cs₂CO₃ (44 mg, 0.14 mmol) in 1,4-dioxane (1.0 mL) was degassed with nitrogen then heated at 90° C. for 18 h. After cooling to room temperature, the reaction mixture was diluted with 10% MeOH in DCM, filtered through a pad of Celite®, and concentrated in vacuo. The residue was purified by preparative HPLC to give the title compound (4.7 mg, 11%). ESI-MS (M+H)+: 463.4, ¹H NMR (400 MHz, DMSO) δ 10.12 (s, 1H), 8.28 (s, 1H), 8.15 (s, 1H), 7.77 (br s, 1H), 7.59 (s, 1H), 7.51 (s, 1H), 7.43-7.33 (m, 4H), 6.98-6.92 (m, 2H), 5.82-5.75 (m, 1H), 4.54 (s, 2H), 1.94-1.86 (m, 1H), 1.50 (d, J=6.6 Hz, 3H), 0.94-0.87 (m, 2H), 0.68-0.62 (m, 2H). The compounds in Table 1 were synthesised using a similar procedure to (R)-1-(3-chlorophenyl)ethyl (6-(((6-cyclopropylimidazo[1,2-a]pyridin-2-yl)methyl)amino)pyrimidin-4-yl)carbamate from 6-chloro-N-((6-cyclopropylimidazo[1,2-a]pyridin-2-yl)methyl)pyrimidin-4-amine and an appropriate alcohol coupling partner.

TABLE 1

| Ex | Compound | Coupling Partner | Analytical Data |
|---|---|---|---|
| 3 | 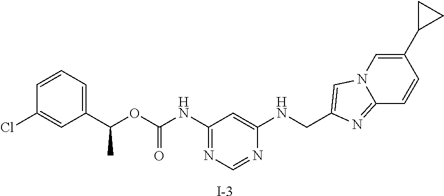 | (S)-1-(3- chlorophenyl)ethan-1- ol | ES-MS (M + H)+: 463.4, ¹H NMR (400 MHz, DMSO) δ 10.16 (s, 1H), 8.33 (s, 1H), 8.21 (s, 1H), 7.83 (br. s, 1H), 7.65 (s, 1H), 7.57 (s, 1H), 7.48-7.39 (m, 4H), 7.04-6.98 (m, 2H), 5.84 (q, J = 6.4 Hz, 1H), 4.60 (s, 2H), 2.00-1.91 (m, 1H), 1.55 (d, J = 6.4 Hz, 3H), 0.99-0.92 (m, 2H), 0.74-0.68 (m, 2H). |
| | (S)-1-(3-chlorophenyl)ethyl (6-(((6- | | |
| | cyclopropylimidazo[1,2-a]pyridin-2- | | |
| | yl)methyl)amino)pyrimidin-4- | | |
| | yl)carbamate (I-3) | | |
| 4 | 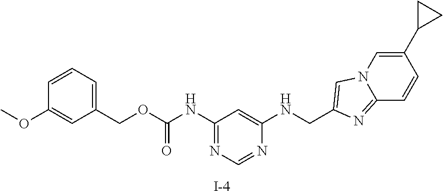 | 3-methoxybenzyl alcohol | ESI-MS (M + H)+: 445.3, ¹H NMR (400 MHz, DMSO) δ 10.15 (s, 1H), 8.30-8.28 (m, 1H), 8.16- 8.15 (m, 1H), 7.82 (br s, 1H), 7.60 (s, 1H), 7.37 (d, J = 9.2 Hz, 1H), 7.29 (t, J = 7.9 Hz, 1H), 7.04-6.93 (m, 4H), 6.89 (q, J = 3.4 Hz, 1H), 5.12 (s, 2H), 4.56 (s, 2H), 3.75 (s, 3H), 1.94-1.86 (m, 1H), 0.93-0.87 (m, 2H), |
| | 3-methoxybenzyl (6-(((6- | | 0.68-0.63 (m, 2H). |
| | cyclopropylimidazo[1,2-a]pyridin-2- | | |
| | yl)methyl)amino)pyrimidin-4- | | |
| | yl)carbamate (I-4) | | |
| 5 | 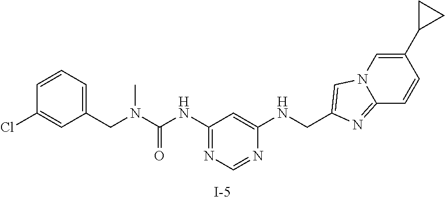 | 3-chloro-N- methylbenzylamine | ESI-MS (M + H)+: 462.3, ¹H NMR (400 MHz, DMSO) δ 8.79 (s, 1H), 8.30-8.28 (m, 1H), 8.14- 8.13 (m, 1H), 7.68 (br s, 1H), 7.60-7.59 (m, 1H), 7.40-7.31 (m, 3H), 7.31- 7.28 (m, 1H), 7.23-7.19 (m, 1H), 7.06 (s, 1H), 6.95 (dd, J = 1.8, 9.3 Hz, 1H), 4.55 (s, 4H), 2.95-2.94 (m, 3H), 1.94-1.86 (m, 1H), |
| | 1-(3-chlorobenzyl)-3-(6-(((6- | | 0.93-0.88 (m, 2H), 0.68- |
| | cyclopropylimidazo[1,2-a]pyridin-2- | | 0.63 (m, 2H). |
| | yl)methyl)amino)pyrimidin-4-yl)-1- | | |
| | methylurea (I-5) | | |
| 6 | 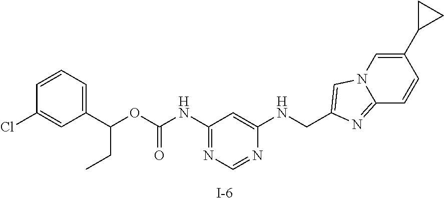 | 1-(3- chlorophenyl)propan- 1-ol | ESI-MS (M + H)+: 477, ¹H NMR (400 MHz, DMSO) δ 10.12 (s, 1H), 8.27 (s, 1H), 8.15 (s, 1H), 7.76 (br s, 1H), 7.58 (s, 1H), 7.46 (s, 1H), 7.43-7.30 (m, 4H), 6.98-6.91 (m, 2H), 5.63- 5.58 (m, 1H), 4.54 (s, 2H), 1.94-1.87 (m, 1H), 1.87- 1.78 (m, 2H), 0.93-0.81 (m, 5H), 0.68-0.61 (m, 2H). |
| | 1-(3-chlorophenyl)propyl (6-(((6- | | |
| | cyclopropylimidazo[1,2-a]pyridin-2- | | |
| | yl)methyl)amino)pyrimidin-4- | | |
| | yl)carbamate (I-6) | | |
| 7 | 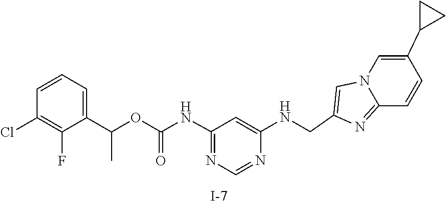 | 1-(3-chloro-2- fluorophenyl)ethan-1- ol | ESI-MS (M + H)+: 481.2, ¹H NMR (400 MHz, DMSO) δ 10.16 (s, 1H), 8.27 (s, 1H), 8.14 (s, 1H), 7.76 (br s, 1H), 7.58 (s, 1H), 7.57-7.45 (m, 2H), 7.35 (d, J = 9.3 Hz, 1H), 7.27 (t, J = 7.8 Hz, 1H), 6.98-6.93 (m, 2H), 5.98 (q, J = 6.6 Hz, 1H), 4.54 (s, 2H), 1.94-1.85 (m, 1H), 1.53 (d, J = 6.6 Hz, 3H), 0.93-0.86 (m, 2H), |
| | 1-(3-chloro-2-fluorophenyl)ethyl (6- | | 0.68-0.62 (m, 2H). |
| | (((6-cyclopropylimidazo[1,2- | | |
| | a]pyridin-2- | | |
| | yl)methyl)amino)pyrimidin-4- | | |
| | yl)carbamate (I-7) | | |

### Example 8

### Synthesis of 5-chloro-2-(1H-tetrazol-1-yl)benzyl (6-(((6-cyclopropylimidazo[1,2-a]pyridin-2-yl)methyl)amino)pyrimidin-4-yl)carbamate (I-8)

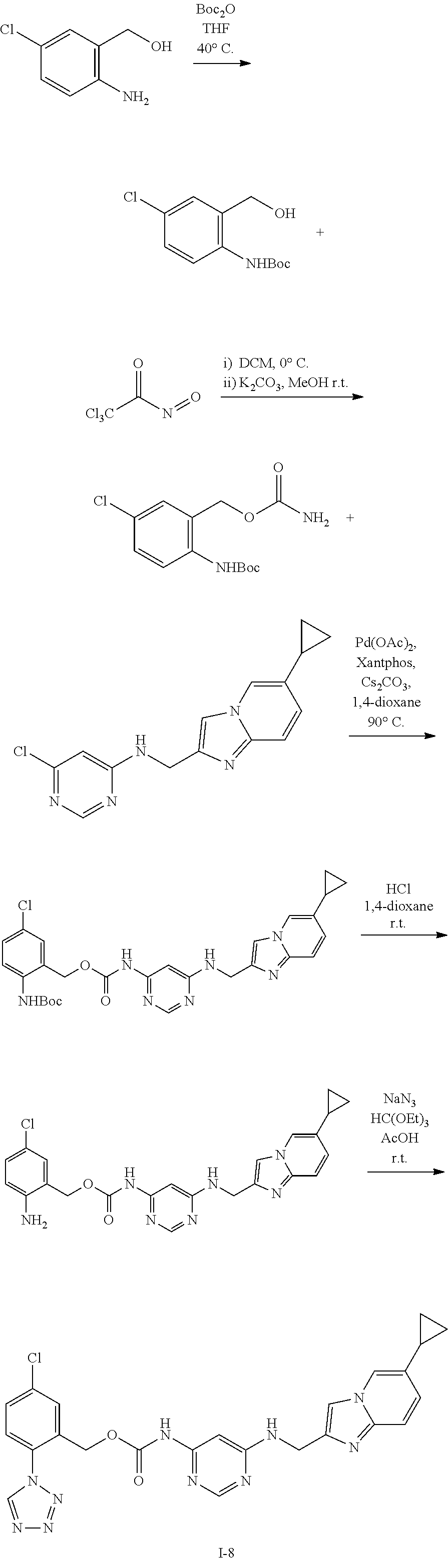

Synthesis of tert-butyl (4-chloro-2-(hydroxymethyl)phenyl)carbamate. (2-Amino-5-chlorophenyl)methanol (0.50 g, 3.2 mmol) was added to a solution of di-tert-butyldicarbonate (0.83 g, 3.8 mmol) in THF (5.0 mL). The reaction mixture was stirred at 40° C. for 2 d. The solvents were removed in vacuo, and the residue was purified by column chromatography on silica gel, eluting with 0-30% EtOAc in cyclohexane to give the title compound, which was used directly in the next step. ¹H NMR (400 MHz, CDCl₃) δ 7.83 (d, J=8.6 Hz, 1H), 7.65 (br s, 1H), 7.23 (dd, J=2.4, 8.7 Hz, 1H), 7.13-7.11 (m, 1H), 4.60 (d, J=5.6 Hz, 2H), 2.62 (t, J=5.6 Hz, 1H), 1.51 (s, 9H).

Synthesis of tert-butyl (2-((carbamoyloxy)methyl)-4-chlorophenyl)carbamate. The title compound was synthesized in a similar manner to (R)-1-(3-chlorophenyl)ethyl carbamate from tert-butyl (4-chloro-2-(hydroxymethyl)phenyl)carbamate and trichloroacetyl isocyanate (0.15 g, 86%). ¹H NMR (400 MHz, CDCl₃) δ 7.83 (s, 1H), 7.76 (d, J=8.1 Hz, 1H), 6.79-6.74 (m, 2H), 5.09 (br s, 2H), 5.02 (s, 2H), 1.51 (s, 9H).

Synthesis of 2-((tert-butoxycarbonyl)amino)-5-chlorobenzyl (6-(((6-cyclopropylimidazo[1,2-a]pyridin-2-yl)methyl)amino)pyrimidin-4-yl)carbamate. The title compound was synthesized in a similar manner to (R)-1-(3-chlorophenyl)ethyl (6-(((6-cyclopropylimidazo[1,2-a]pyridin-2-yl)methyl)amino)pyrimidin-4-yl)carbamate, starting from tert-butyl (2-((carbamoyloxy)methyl)-4-chlorophenyl)carbamate and 6-chloro-N-((6-cyclopropylimidazo[1,2-a]pyridin-2-yl)methyl)pyrimidin-4-amine, and then used directly in the next step (0.21 g, 74%).

Synthesis of 2-amino-5-chlorobenzyl (6-(((6-cyclopropylimidazo[1,2-a]pyridin-2-yl)methyl)amino)pyrimidin-4-yl)carbamate. HCl (4.0 M in 1,4-dioxane, 20 μL, 0.080 mmol) was added to a solution of 2-((tert-butoxycarbonyl)amino)-5-chlorobenzyl-(6-(((6-cyclopropylimidazo[1,2-a]pyridin-2-yl)methyl)amino)pyrimidin-4-yl)carbamate (60 mg, 0.053 mmol) in 1,4-dioxane (0.5 mL). The mixture was stirred at room temperature for 4 h, then quenched by addition of NaHCO₃ (sat. aq.) and extracted with DCM. The combined organics were passed through a hydrophobic frit, then concentrated in vacuo to give the title compound, which was used in the next step without further purification.

Synthesis of 5-chloro-2-(1H-tetrazol-1-yl)benzyl (6-(((6-cyclopropylimidazo[1,2-a]pyridin-2-yl)methyl)amino)pyrimidin-4-yl)carbamate. NaN₃ (4.4 mg, 0.067 mmol) and triethylorthoformate (29 μL, 0.17 mmol) were added to a solution of 2-amino-5-chlorobenzyl (6-(((6-cyclopropylimidazo[1,2-a]pyridin-2-yl)methyl)amino)pyrimidin-4-yl)carbamate (25 mg, 0.054 mmol) in acetic acid (50 μL). The mixture was stirred at room temperature for 18 h. The reaction was diluted with water and extracted with EtOAc. The combined organics were passed through a hydrophobic frit and the solvent was removed in vacuo. The residue was purified by preparative HPLC to give the title compound (11 mg, 39%). ESI-MS (M+H)+: 517.3, ¹H NMR (400 MHz, DMSO) δ 10.22 (s, 1H), 9.89 (s, 1H), 8.29-8.29 (m, 1H), 8.16 (s, 1H), 7.91 (d, J=1.9 Hz, 1H), 7.84 (br s, 1H), 7.77-7.70 (m, 2H), 7.60 (s, 1H), 7.36 (d, J=9.4 Hz, 1H), 6.95 (dd, J=1.8, 9.3 Hz, 2H), 5.04 (s, 2H), 4.55 (s, 2H), 1.94-1.86 (m, 1H), 0.93-0.87 (m, 2H), 0.68-0.63 (m, 2H).

### Example 9

### Synthesis of 2-(3-chlorophenyl)-2-hydroxyethyl (6-(((6-cyclopropylimidazo[1,2-a]pyridin-2-yl)methyl)amino pyrimidin-4-yl)carbamate (I-9)

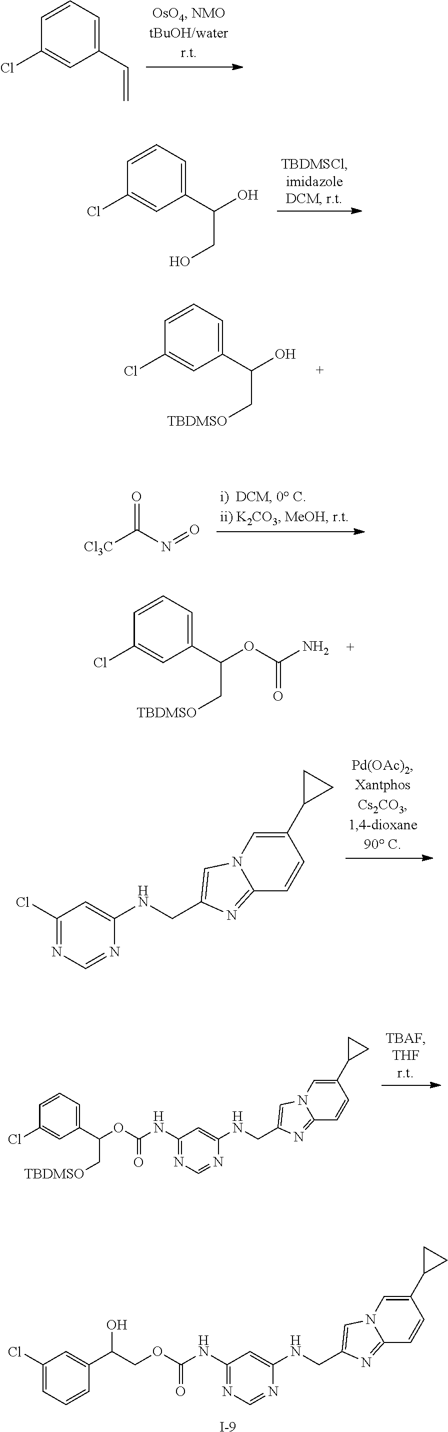

Synthesis of 1-(3-chlorophenyl)ethane-1,2-diol. OsO₄ (2.5% in tBuOH, 0.45 mL, 0.036 mmol) was added dropwise to a solution of 3-chlorostyrene (1.0 g, 7.2 mmol) and N-methyl-morpholine oxide (1.3 g, 11 mmol) in water (9.0 mL) and tBuOH (27 mL). The mixture was stirred at room temperature for 18 h. Na₂SO₃ (1.0 M aq., 20 mL) was added, and the mixture was stirred for 30 min, then extracted with DCM. The combined organics were dried over MgSO₄ and concentrated in vacuo to give the title compound, which was used without further purification. ¹H NMR (400 MHz, CDCl₃) δ 7.38 (s, 1H), 7.33-7.21 (m, 3H), 4.79 (dd, J=3.4, 8.0 Hz, 1H), 3.76 (dd, J=3.4, 11.2 Hz, 1H), 3.70 (t, J=4.7 Hz, 1H), 3.65-3.59 (m, 1H), one exchangeable proton not visible.

Synthesis of 2-((tert-butyldimethylsilyl)oxy)-1-(3-chlorophenyl)ethan-1-ol. Imidazole (0.54 g, 7.9 mmol) and tert-butyldimethylsilyl chloride (1.2 g, 7.9 mmol) were added to a solution of 1-(3-chlorophenyl)ethane-1,2-diol (1.3 g, 7.2 mmol) in DCM (35 mL). The mixture was stirred at room temperature for 18 h. The mixture was filtered, then concentrated in vacuo. The residue was purified by silica gel column chromatography, eluting with a gradient of 0-20% EtOAc in cyclohexane to give the title compound (1.5 g, 70%). ¹H NMR (400 MHz, CDCl₃) δ 7.38 (s, 1H), 7.31-7.20 (m, 3H), 4.72 (dd, J=3.5, 8.3 Hz, 1H), 3.76 (dd, J=3.5, 10.1 Hz, 1H), 3.52 (dd, J=8.5, 10.0 Hz, 1H), 0.91 (s, 9H), 0.06 (d, J=3.3 Hz, 6H), exchangeable proton not visible.

Synthesis of 2-((tert-butyldimethylsilyl)oxy)-1-(3-chlorophenyl)ethyl carbamate. The title compound was synthesized in a similar manner to (R)-1-(3-chlorophenyl)ethyl carbamate, starting from 2-((tert-butyldimethylsilyl)oxy)-1-(3-chlorophenyl)ethan-1-ol and trichloroacetyl isocyanate (0.35 g, 61%). ¹H NMR (400 MHz, CDCl₃) δ 7.34 (s, 1H), 7.29-7.18 (m, 3H), 5.69-5.64 (m, 1H), 4.68 (br s, 2H), 3.87-3.75 (m, 2H), 0.87-0.85 (m, 9H), 0.00 (d, J=1.9 Hz, 6H).

Synthesis of 2-((tert-butyldimethylsilyl)oxy)-1-(3-chlorophenyl)ethyl (6-(((6-cyclopropylimidazo[1,2-a]pyridin-2-yl)methyl)amino)pyrimidin-4-yl)carbamate. The title compound was synthesized in a similar manner to (R)-1-(3-chlorophenyl)ethyl (6-(((6-cyclopropylimidazo[1,2-a]pyridin-2-yl)methyl)amino)pyrimidin-4-yl)carbamate, starting from 2-((tert-butyldimethylsilyl)oxy)-1-(3-chlorophenyl)ethyl carbamate and 6-chloro-N-((6-cyclopropylimidazo[1,2-a]pyridin-2-yl)methyl)pyrimidin-4-amine (0.25 g, quant.). ESI-MS (M+H)+: 593.3.

Synthesis of 2-(3-chlorophenyl)-2-hydroxyethyl (6-(((6-cyclopropylimidazo[1,2-a]pyridin-2-yl)methyl)amino)pyrimidin-4-yl)carbamate. TBAF (3.3 M in THF, 0.15 mL, 0.51 mmol) was added to a solution of 2-((tert-butyldimethylsilyl)oxy)-1-(3-chlorophenyl)ethyl (6-(((6-cyclopropylimidazo[1,2-a]pyridin-2-yl)methyl)amino)pyrimidin-4-yl)carbamate (0.20 g, 0.10 mmol) in THF (2.0 mL). The mixture was stirred at room temperature for 1 h. Water was added and the mixture was extracted with DCM. The organic extract was passed through a hydrophobic frit and concentrated in vacuo. The residue was purified by reverse phase HPLC to give the product as an 85:15 mixture of isomers (3 mg, 6%). ESI-MS (M+H)+: 479.3, ¹H NMR (Major Isomer) (400 MHz, DMSO) δ 9.95 (s, 1H), 8.23-8.19 (m, 1H), 8.08 (s, 1H), 7.71 (s, 1H), 7.55-7.51 (m, 1H), 7.41-7.38 (m, 1H), 7.30-7.26 (m, 4H), 6.92-6.86 (m, 2H), 5.68 (s, 1H), 4.77-4.73 (m, 1H), 4.48 (s, 2H), 4.06 (d, J=5.6 Hz, 2H), 1.87-1.79 (m, 1H), 0.87-0.80 (m, 2H), 0.61-0.57 (m, 2H).

### Example 10

### Synthesis of 1-(5-chloro-2-(1H-tetrazol-1-yl)benzyl)-3-(4-(((6-cyclopropylimidazo[1,2-a]pyridin-2-yl)methyl)amino)pyridin-2-yl)urea (I-10)

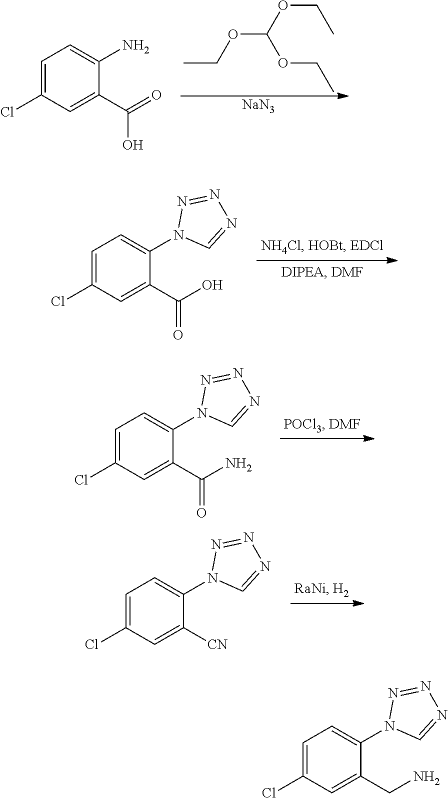

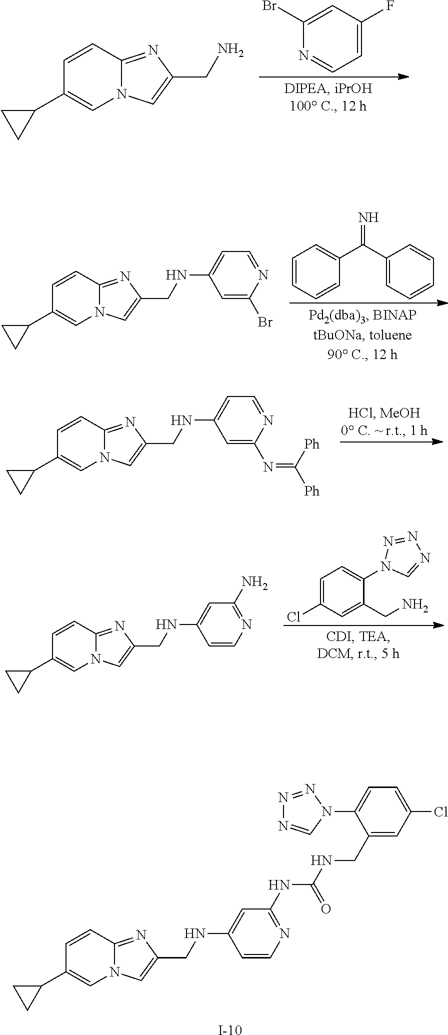

### Synthesis of 5-chloro-2-(1H-tetrazol-1-yl)benzoic acid

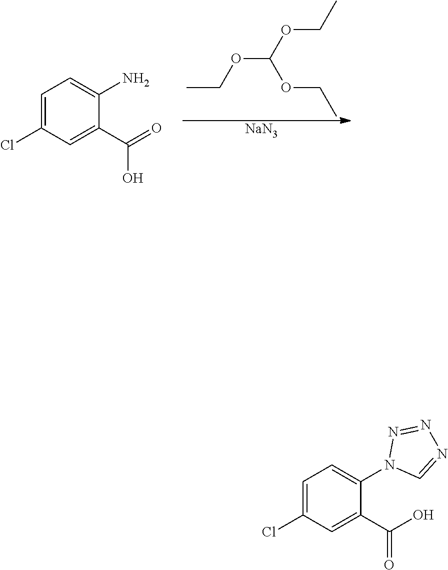

To a mixture of 2-amino-5-chlorobenzoic acid (100 mg, 0.58 mmol) and NaN₃ (38 mg, 0.58 mmol) in triethoxymethane (3 mL) was added P₂O₅ (16 mg, 0.12 mmol). The mixture was stirred at 130° C. for 3 h. After the reaction was cooled to room temperature, water (20 mL) was added, and then the mixture was extracted with EtOAc (20 mL×3). The combined organics were dried over Na₂SO₄ and concentrated in vacuo to give the crude product, which was purified by recrystallization from DCM to give 5-chloro-2-(1H-tetrazol-1-yl)benzoic acid (55 mg, 42%) as a yellow solid. ESI-MS [M+H]+: 225.1.

### Synthesis of 5-chloro-2-(1H-tetrazol-1-yl)benzamide

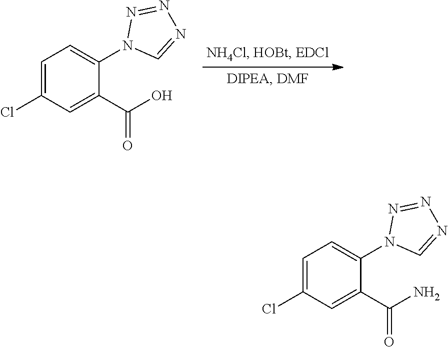 [SMILES: O=C(O)c1cc(Cl)ccc1-n1cnnn1 ; NC(=O)c1cc(Cl)ccc1-n1cnnn1]

To a solution of 5-chloro-2-(1H-tetrazol-1-yl)benzoic acid (55 mg, 0.24 mmol), NH₄Cl (26 mg, 0.49 mmol), HOBt (66 mg, 0.49 mmol), and EDCI (94 mg, 0.49 mmol) in DMF (5 mL) was added DIPEA (127 mg, 0.98 mmol). The mixture was stirred at 25° C. for 16 h, and then water (30 mL) was added. The mixture was extracted with EtOAc (20 mL×3) and the combined organics were dried over Na₂SO₄, filtered, and concentrated in vacuo to give 5-chloro-2-(1H-tetrazol-1-yl)benzamide (40 mg, 73%) as a yellow oil. ESI-MS [M+H]+: 224.0.

### Synthesis of 5-chloro-2-(1H-tetrazol-1-yl)benzonitrile

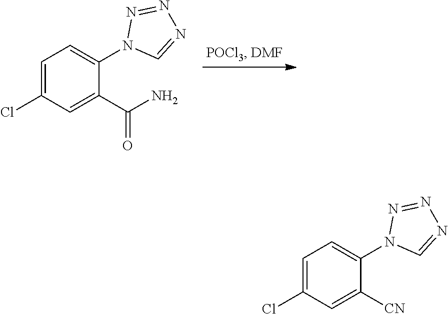 [SMILES: NC(=O)c1cc(Cl)ccc1-n1cnnn1 ; N#Cc1cc(Cl)ccc1-n1cnnn1]

To a mixture of 5-chloro-2-(1H-tetrazol-1-yl)benzamide (40 mg, 0.18 mmol) in DMF (2 mL) was added POCl₃ (165 mg, 1.08 mmol) at 0° C. The mixture was stirred at 25° C. for 16 h. Water (20 mL) was added, and then the mixture was extracted with DCM (10 mL×3). The combined organic layers were washed with brine, dried over Na₂SO₄, and concentrated in vacuo to give 5-chloro-2-(1H-tetrazol-1-yl)benzonitrile (30 mg, 81%) as a oil. ESI-MS [M+H]+: 206.1.

### Synthesis of (5-chloro-2-(1H-tetrazol-1-yl)phenyl)methanamine

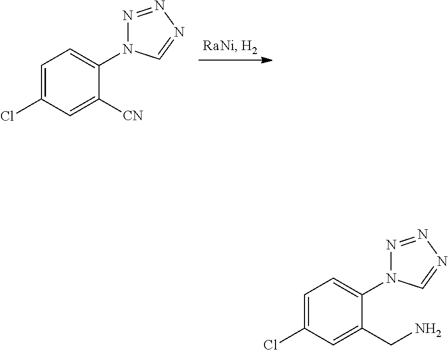 [SMILES: N#Cc1cc(Cl)ccc1-n1cnnn1 ; NCc1cc(Cl)ccc1-n1cnnn1]

To a mixture of 5-chloro-2-(1H-tetrazol-1-yl)benzonitrile (30 mg, 0.15 mmol) in NH₃ in MeOH (2 mL, 7M in MeOH) was added RaNi (30 mg). The mixture was stirred at 25° C. for 1 h under H₂, whereupon it was filtered and the filter cake was washed with MeOH (10 mL). The filtrate was concentrated in vacuo to give (5-chloro-2-(1H-tetrazol-1-yl)phenyl)methanamine (21 mg, 67%) as a yellow oil, which was used in the next step without purification. ESI-MS [M+H]+: 210.1.

### Synthesis of 2-bromo-N-((6-cyclopropylimidazo[1,2-a]pyridin-2-yl)methyl pyridin-4-amine

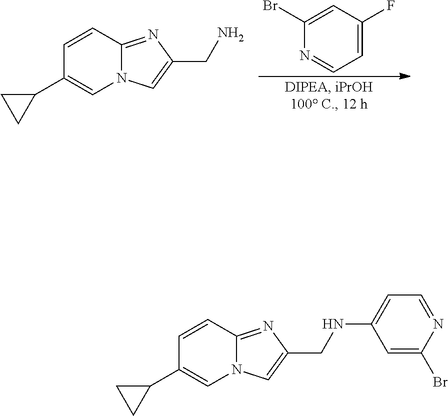 [SMILES: NCc1cn2cc(C3CC3)ccc2n1 ; Fc1ccnc(Br)c1 ; Brc1cc(NCc2cn3cc(C4CC4)ccc3n2)ccn1]

A mixture of (6-cyclopropylimidazo[1,2-a]pyridin-2-yl)methanamine (935 mg, 5 mmol), 2-bromo-4-fluoropyridine (1.1 g, 6.25 mmol) and DIPEA (1.29 g, 10 mmol) in iPrOH (30 mL) was stirred at 100° C. for 12 h. The reaction was cooled to room temperature, then concentrated in vacuo to give the crude, which was purified with silica gel column chromatography (eluent: PE/EtOAc=1/1) to give 2-bromo-N-((6-cyclopropylimidazo[1,2-a]pyridin-2-yl)methyl)pyridin-4-amine (1 g, 58%) as a yellow solid. ESI-MS [M+H]+: 344.2

### Synthesis of N-((6-cyclopropylimidazo[1,2-a]pyridin-2-yl)methyl)-2-((diphenylmethylene)amino)pyridin-4-amine

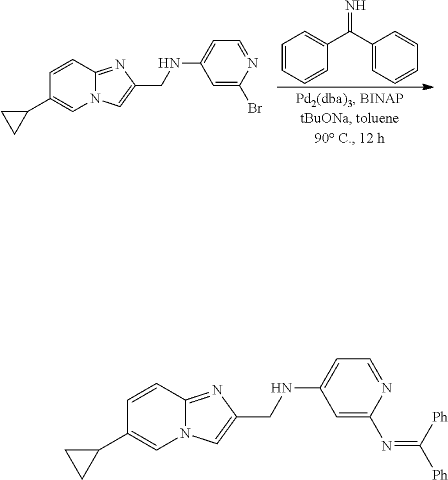

A mixture of 2-bromo-N-((6-cyclopropylimidazo[1,2-a]pyridin-2-yl)methyl)pyridin-4-amine (1 g, 2.9 mmol), diphenylmethanimine (1.1 g, 5.8 mmol), Pd₂(dba)₃ (265 mg, 0.29 mmol), BINAP (360 mg, 0.58 mmol), and tBuONa (835 mg, 8.7 mmol) in toluene (25 mL) was stirred at 90° C. for 12 h. The reaction was diluted with water (40 mL), then extracted with EtOAc (40 mL×3). The combined organic layers were washed with brine (40 mL), dried over anhydrous Na₂SO₄, and concentrated in vacuo to give the crude product, which was purified with silica gel column chromatography (eluent: PE/EtOAc=2/1) to give N-((6-cyclopropylimidazo[1,2-a]pyridin-2-yl)methyl)-2-((diphenylmethylene)amino)pyridin-4-amine (760 mg, 59%) as a yellow solid. ESI-MS [M+H]+: 444.1.

### Synthesis of N⁴-((6-cyclopropylimidazo[1,2-a]pyridin-2-yl)methyl)pyridine-2,4-diamine

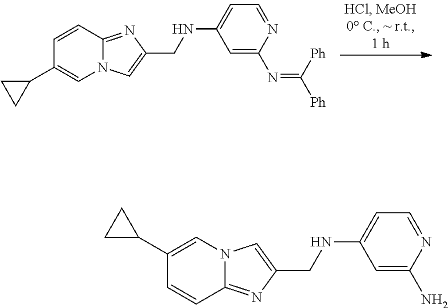 [SMILES: c1ccc(C(=Nc2cc(NCc3cn4cc(C5CC5)ccc4n3)ccn2)c2ccccc2)cc1 ; Nc1cc(NCc2cn3cc(C4CC4)ccc3n2)ccn1]

To a solution of N-((6-cyclopropylimidazo[1,2-a]pyridin-2-yl)methyl)-2-((diphenylmethylene)amino)pyridin-4-amine (443 mg, 1 mmol) in MeOH (10 mL) was added HCl (2 mL, 4 M solution in MeOH) at 0° C. The resulting reaction was stirred at room temperature for 1 h. The reaction was concentrated in vacuo to give the crude product, which was neutralized with NH₃ (5 mL, 7 M solution in MeOH) and concentrated in vacuo. The crude product was purified with silica gel column chromatography (eluent: DCM/MeOH=10/1) to give N⁴-((6-cyclopropylimidazo[1,2-a]pyridin-2-yl)methyl)pyridine-2,4-diamine (260 mg, 93%) as a yellow solid. ESI-MS [M+H]+: 280.2.

### Synthesis of 1-(5-chloro-2-(1H-tetrazol-1-yl)benzyl)-3-(4-(((6-cyclopropylimidazo[1,2-a]pyridin-2-yl)methyl)amino)pyridin-2-yl)urea

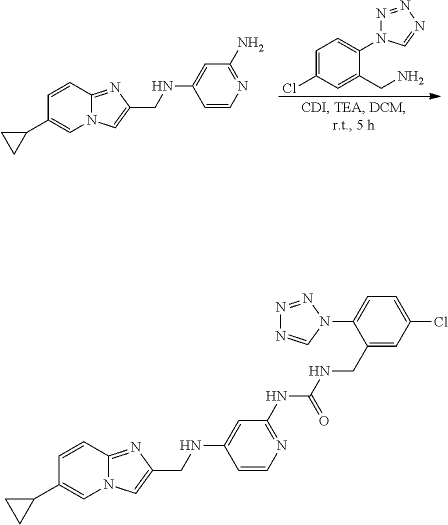 [SMILES: Nc1cc(NCc2cn3cc(C4CC4)ccc3n2)ccn1 ; NCc1cc(Cl)ccc1-n1cnnn1 ; O=C(NCc1cc(Cl)ccc1-n1cnnn1)Nc1cc(NCc2cn3cc(C4CC4)ccc3n2)ccn1]

A solution of N⁴-((6-cyclopropylimidazo[1,2-a]pyridin-2-yl)methyl)pyridine-2,4-diamine (28 mg, 0.1 mmol), di(1H-imidazol-1-yl)methanone (32 mg, 0.2 mmol), and triethylamine (50 mg, 0.5 mmol) in DCM (1.0 mL) was stirred at room temperature for 3 h. Then, a solution of (5-chloro-2-(1H-tetrazol-1-yl)phenyl)methanamine (42 mg, 0.2 mmol) in DCM (0.5 mL) was added. The mixture was stirred at room temperature for another 2 h. The reaction mixture was concentrated in vacuo to give the crude, which was purified by Prep-TLC (eluent: DCM/MeOH=20/1) to give 1-(5-chloro-2-(1H-tetrazol-1-yl)benzyl)-3-(4-(((6-cyclopropylimidazo[1,2-a]pyridin-2-yl)methyl)amino)pyridin-2-yl)urea (10.9 mg, 21%) as a white solid. ESI-MS [M+H]⁺: 515.2. ¹H NMR (400 MHz, DMSO) δ 9.85 (s, 1H), 9.35 (s, 1H), 8.96 (s, 1H), 8.27 (s, 1H), 7.60-7.57 (m, 5H), 7.35 (d, J=9.3 Hz, 1H), 7.10 (s, 1H), 6.94 (dd, J=9.3, 1.6 Hz, 1H), 6.27-6.21 (m, 2H), 4.29-4.23 (m, 4H), 1.91-1.85 (m, 1H), 0.88-0.85 (m, 2H), 0.65-0.61 (m, 2H).

### Example 11

### Synthesis of 5-chloro-2-(1H-tetrazol-1-yl)benzyl (4-(((6-cyclopropylimidazo[1,2-a]pyridin-2-yl)methyl)amino)pyridin-2-yl)carbamate (I-11)

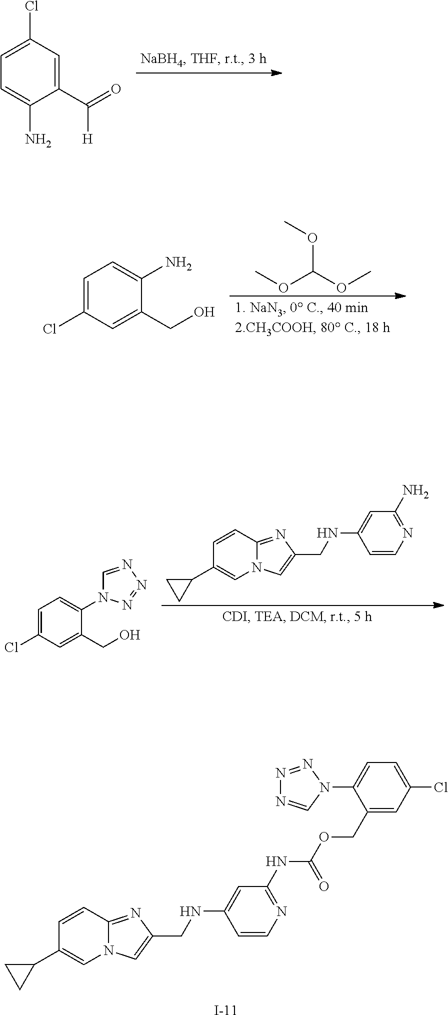

### Synthesis of (2-amino-5-chlorophenyl)methanol

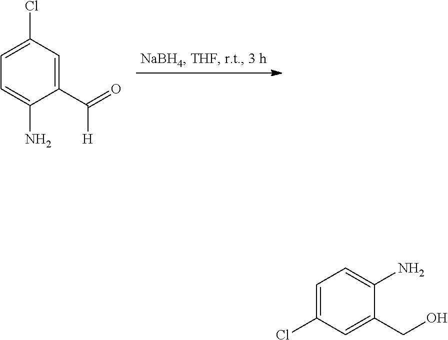 [SMILES: Nc1ccc(Cl)cc1C=O ; Nc1ccc(Cl)cc1CO]

To a solution of 2-amino-5-chlorobenzaldehyde (0.50 g, 3.2 mmol) in THF (10 mL) was added NaBH₄ (0.18 g, 4.8 mmol), and the resulting mixture was stirred at room temperature for 3 h. The reaction mixture was quenched with water (30 mL), extracted with EtOAc (30 mL×3), and washed with brine (40 mL×2). The combined organic phase was dried over Na₂SO₄ and concentrated in vacuo to give the crude product, which was purified by column chromatography (eluent: PE/EtOAc=2/1) to afford (2-amino-5-chlorophenyl)methanol (0.40 g, 80%) as a yellow solid. ESI-MS: [M+H]+, 156.1

### Synthesis of (5-chloro-2-(1H-tetrazol-1-yl)phenyl)methanol

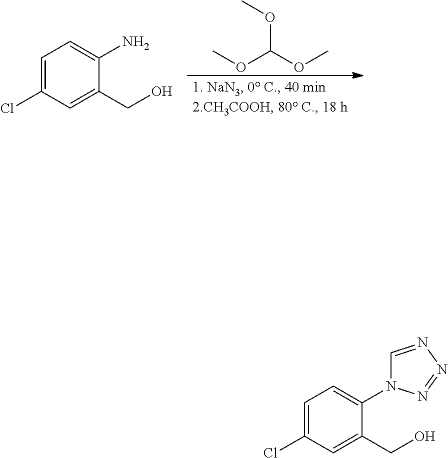

To a solution of (2-amino-5-chlorophenyl)methanol (0.36 g, 2.3 mmol) in trimethoxymethane (5 mL) was added NaN₃ (0.37 g, 5.7 mmol) at 0° C. The mixture was stirred at 0° C. for 40 min. Then, acetic acid (0.05 mL) was added, and the mixture was stirred at 80° C. for 16 h. The reaction mixture was quenched with water (20 mL), extracted with EtOAc (20 mL×3), and washed with brine (30 mL×2). The combined organic phase was dried over Na₂SO₄ and concentrated in vacuo to give the crude product, which was purified by column chromatography (eluent: PE/EtOAc=1/1) to afford (5-chloro-2-(1H-tetrazol-1-yl)phenyl)methanol (0.21 g, 43%) as a yellow solid. ESI-MS: [M+H]+, 211.1

### Synthesis of 5-chloro-2-(1H-tetrazol-1-yl)benzyl (4-(((6-cyclopropylimidazo[1,2-a]pyridin-2-yl)methyl)amino)pyridin-2-yl)carbamate

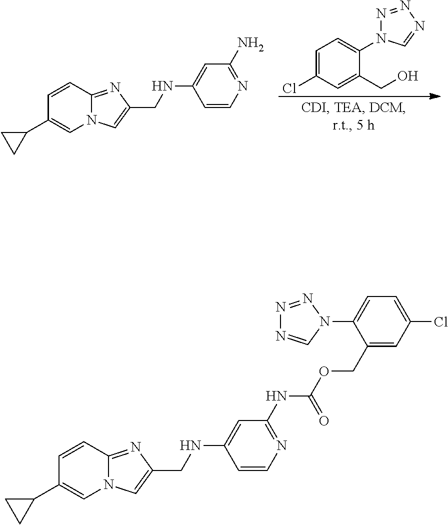 [SMILES: Nc1cc(NCc2cn3cc(C4CC4)ccc3n2)ccn1 ; OCc1cc(Cl)ccc1-n1cnnn1 ; O=C(Nc1cc(NCc2cn3cc(C4CC4)ccc3n2)ccn1)OCc1cc(Cl)ccc1-n1cnnn1]

A solution of N⁴-((6-cyclopropylimidazo[1,2-a]pyridin-2-yl)methyl)pyridine-2,4-diamine (35 mg, 0.13 mmol), di(1H-imidazol-1-yl)methanone (41 mg, 0.25 mmol), and triethylamine (64 mg, 0.63 mmol) in DCM (1.5 mL) was stirred at room temperature for 3 h. Then, a solution of (5-chloro-2-(1H-tetrazol-1-yl)phenyl)methanol (53 mg, 0.25 mmol) in DCM (0.5 mL) was added. The mixture was stirred at room temperature for another 2 h. The reaction mixture was concentrated in vacuo to give the crude product, which was purified by Prep-TLC (eluent: DCM/MeOH=12/1) to give 5-chloro-2-(1H-tetrazol-1-yl) benzyl (4-(((6-cyclopropylimidazo[1,2-a]pyridin-2-yl)methyl)amino)pyridin-2-yl)carbamate (11.9 mg, 18%) as a white solid. ESI-MS [M+H]⁺: 516.2, ¹H NMR (400 MHz, DMSO) δ 9.94-9.80 (m, 2H), 8.28 (s, 1H), 7.88 (s, 1H), 7.73-7.68 (m, 3H), 7.60 (s, 1H), 7.35 (d, J=9.3 Hz, 1H), 7.12 (t, J=5.5 Hz, 1H), 7.03 (s, 1H), 6.94 (dd, J=9.3, 1.4 Hz, 1H), 6.28 (dd, J=5.7, 1.8 Hz, 1H), 4.98 (s, 2H), 4.32 (d, J=5.5 Hz, 2H), 1.91-1.85 (m, 1H), 0.90-0.85 (m, 2H), 0.65-0.61 (m, 2H).

### Example 12

### Synthesis of 5-chloro-2-(1H-tetrazol-1-yl)benzyl (4-((6-cyclopropylimidazo[1,2-a]pyridin-2-yl)methoxy)pyridin-2-yl)carbamate (I-12)

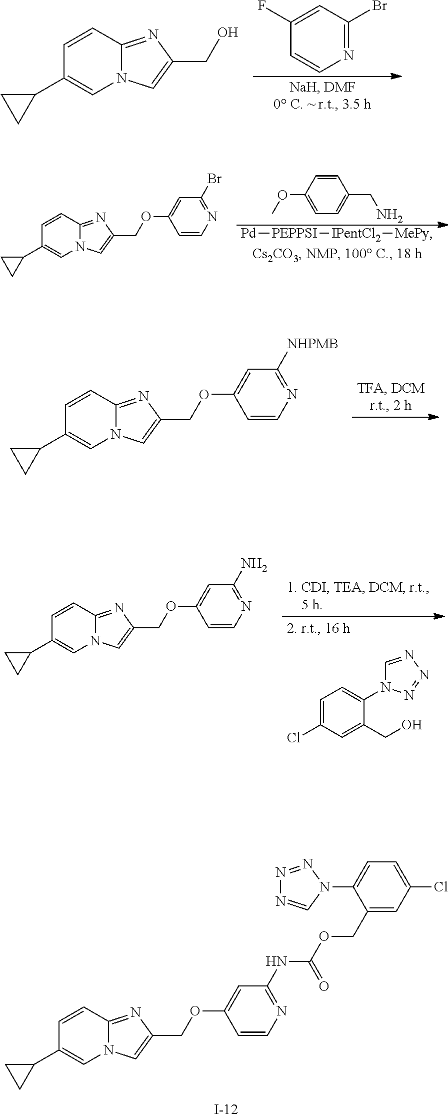

### Synthesis of 2-(((2-bromopyridin-4-yl)oxy)methyl)-6-cyclopropylimidazo[1,2-a]pyridine

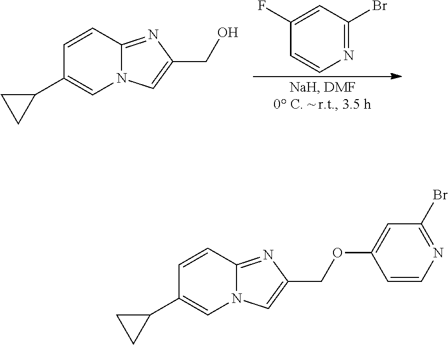 [SMILES: OCc1cn2cc(C3CC3)ccc2n1 ; Fc1ccnc(Br)c1 ; Brc1cc(OCc2cn3cc(C4CC4)ccc3n2)ccn1]

To a solution of (6-cyclopropylimidazo[1,2-a]pyridin-2-yl)methanol (0.50 g, 2.7 mmol) in DMF (5 mL) was added NaH (0.32 g, 60% in mineral oil, 8.0 mmol) at 0° C., and the mixture was stirred at 0° C. for 0.5 h. Then a solution of 2-bromo-4-fluoropyridine (0.93 g, 5.3 mmol) in DMF (1 mL) was added, and the mixture was stirred at room temperature for 3 h. The reaction was quenched with water (50 mL) and extracted with EtOAc (50 mL×3). The combined organic phase was washed with brine (30 mL×2), dried over anhydrous Na₂SO₄, and concentrated in vacuo to give the crude product, which was purified by column chromatography (eluent: DCM/MeOH=50/1) to afford 2-(((2-bromopyridin-4-yl)oxy)methyl)-6-cyclopropylimidazo[1,2-a]pyridine (0.65 g, 70%) as a yellow oil. ESI-MS: [M+H]⁺, 344.1

### Synthesis of 4-((6-cyclopropylimidazo[1,2-a]pyridin-2-yl)methoxy)-N-(4-methoxybenzyl)pyridin-2-amine

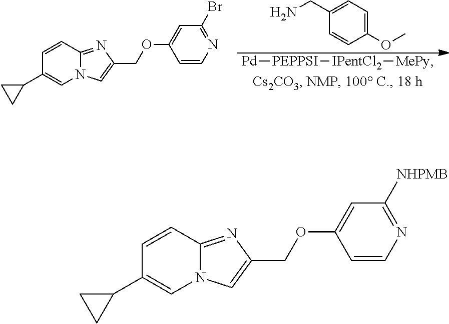

A mixture of 2-(((2-bromopyridin-4-yl)oxy)methyl)-6-cyclopropylimidazo[1,2-a]pyridine (0.65 g, 1.9 mmol), (4-methoxyphenyl)methanamine (0.78 g, 5.9 mmol), Pd-PEPPSI-IPentCl₂-MePy (0.16 g, 0.19 mmol), and Cs₂CO₃ (1.9 g, 5.9 mmol) in NMP (10 mL) was stirred at 100° C. for 18 h under N₂. The reaction mixture was filtered through Celite®, and the filter cake was washed with DCM/MeOH (10/1, 100 mL). The filtrate was concentrated in vacuo to give the crude, which was purified by column chromatography (eluent: DCM/MeOH=25/1) to afford 4-((6-cyclopropylimidazo[1,2-a]pyridin-2-yl)methoxy)-N-(4-methoxybenzyl)pyridin-2-amine (0.70 g, 92%) as a yellow oil. ESI-MS: [M+H]+, 401.2

### Synthesis of 4-((6-cyclopropylimidazo[1,2-a]pyridin-2-yl)methoxy)pyridin-2-amine

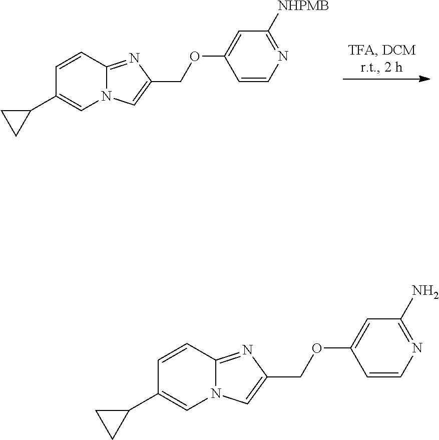

To a solution of 4-((6-cyclopropylimidazo[1,2-a]pyridin-2-yl)methoxy)-N-(4-methoxybenzyl)pyridin-2-amine (0.70 g, 1.8 mmol) in DCM (10 mL) was added TFA (10 mL) at 0° C., and the mixture was stirred at room temperature for 2 h. The reaction was quenched with saturated aqueous NaHCO₃ (50 mL), extracted with EtOAc (30 mL×3), and washed with brine (100 mL×2). The combined organic layers were dried over anhydrous Na₂SO₄ and concentrated in vacuo to give the crude, which was purified by column chromatography (eluent: DCM/MeOH=10/1) to afford 4-((6-cyclopropylimidazo[1,2-a]pyridin-2-yl)methoxy)pyridin-2-amine (0.50 g, quant) as a brown solid. ESI-MS: [M+H]+, 281.2.

### Synthesis of 5-chloro-2-(1H-tetrazol-1-yl)benzyl (4-((6-cyclopropylimidazo[1,2-a]pyridin-2-yl)methoxy)pyridin-2-yl)carbamate

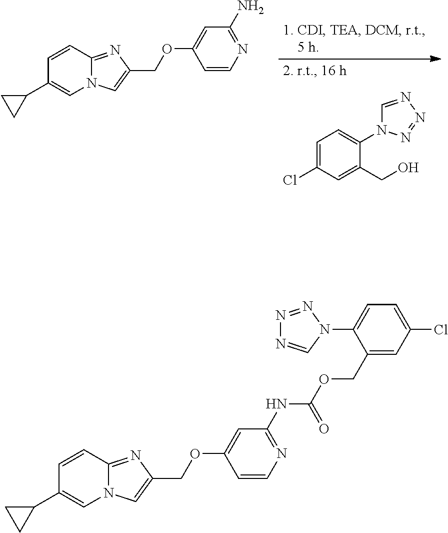 [SMILES: Nc1cc(OCc2cn3cc(C4CC4)ccc3n2)ccn1 ; OCc1cc(Cl)ccc1-n1cnnn1 ; O=C(Nc1cc(OCc2cn3cc(C4CC4)ccc3n2)ccn1)OCc1cc(Cl)ccc1-n1cnnn1]

A mixture of 4-((6-cyclopropylimidazo[1,2-a]pyridin-2-yl)methoxy)pyridin-2-amine (50 mg, 0.18 mmol), di(1H-imidazol-1-yl)methanone (43 mg, 0.27 mmol), and triethylamine (54 mg, 0.54 mmol) in DCM (5 mL) was stirred at room temperature for 5 h, then (5-chloro-2-(1H-tetrazol-1-yl)phenyl)methanol (75 mg, 0.36 mmol) was added and stirred at room temperature for 16 h. The reaction mixture was concentrated in vacuo to give the crude, which was purified by Prep-TLC (eluent: DCM/MeOH=10/1) to afford 5-chloro-2-(1H-tetrazol-1-yl)benzyl (4-((6-cyclopropylimidazo[1,2-a]pyridin-2-yl)methoxy)pyridin-2-yl)carbamate (10 mg, 11%) as a white solid. ESI-MS: [M+H]+, 517.2; ¹H NMR (400 MHz, DMSO) δ 10.31 (s, 1H), 9.90 (s, 1H), 8.35 (s, 1H), 8.10 (d, J=5.8 Hz, 1H), 7.93 (d, J=1.8 Hz, 1H), 7.88 (s, 1H), 7.78-7.70 (m, 2H), 7.45-7.41 (m, 2H), 7.03-6.99 (m, 1H), 6.83-6.79 (m, 1H), 5.23 (s, 2H), 5.06 (s, 2H), 1.96-1.91 (m, 1H), 0.96-0.89 (m, 2H), 0.72-0.66 (m, 2H).

### Example 13

### Synthesis of 3-chlorobenzyl (4-(((6-cyclopropylimidazo[1,2-a]pyridin-2-yl)methyl)amino)pyridin-2-yl)carbamate (I-13)

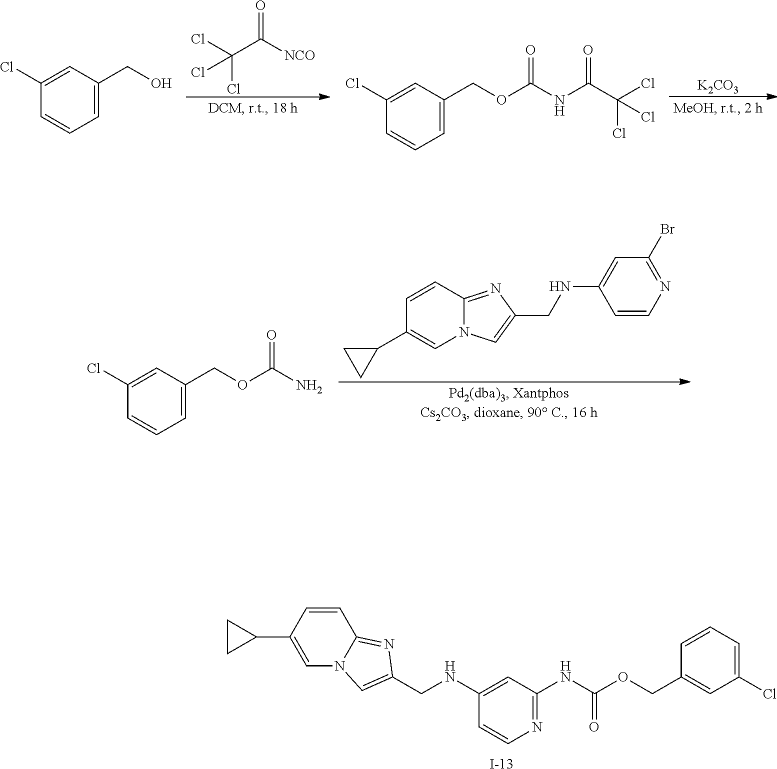

### Synthesis of 3-chlorobenzyl (2,2,2-trichloroacetyl)carbamate

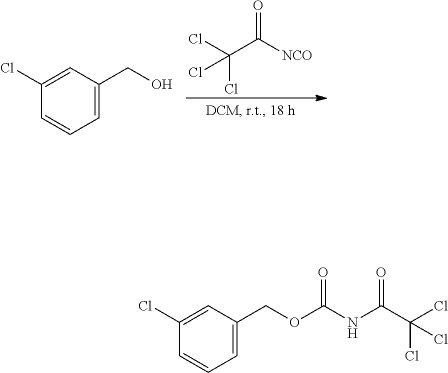

To a solution of (3-chlorophenyl)methanol (0.8 g, 5.6 mmol) in DCM (10 mL) was added 2,2,2-trichloroacetyl isocyanate (1.27 g, 6.8 mmol) at room temperature, and the mixture was stirred at room temperature for 18 h. The mixture was quenched with water (50 mL) and extracted with EtOAc (30 mL×3). The combined organic layers were washed with brine (30 mL), dried over Na₂SO₄, and concentrated in vacuo to afford 3-chlorobenzyl (2,2,2-trichloroacetyl)carbamate (1.5 g, crude) as white solid, which was used in the next step directly. ESI-MS [M+H]+: 329.9

### Synthesis of 3-chlorobenzyl carbamate

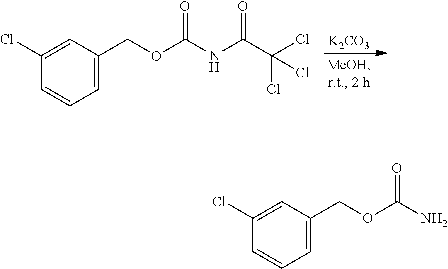

A mixture of 3-chlorobenzyl (2,2,2-trichloroacetyl)carbamate (1.5 g, crude) and K₂CO₃ (1.9 g, 13.8 mmol) in MeOH (10 mL) was stirred at room temperature for 2 h. The mixture was quenched with water (50 mL) and extracted with EtOAc (30 mL×3). The combined organic layers were washed with brine (30 mL), dried over Na₂SO₄, and concentrated in vacuo. The residue was purified by flash column chromatography (eluent: EtOAc/PE from 0 to 30%) to give 3-chlorobenzyl carbamate (700 mg, 67% for 2 steps) as a white solid. ESI-MS [M+H]+: 186.0

### Synthesis of 1-(3-chlorophenyl)ethyl (4-(((6-cyclopropylimidazo[1,2-a]pyridin-2-yl)methyl)amino)pyridin-2-yl)carbamate

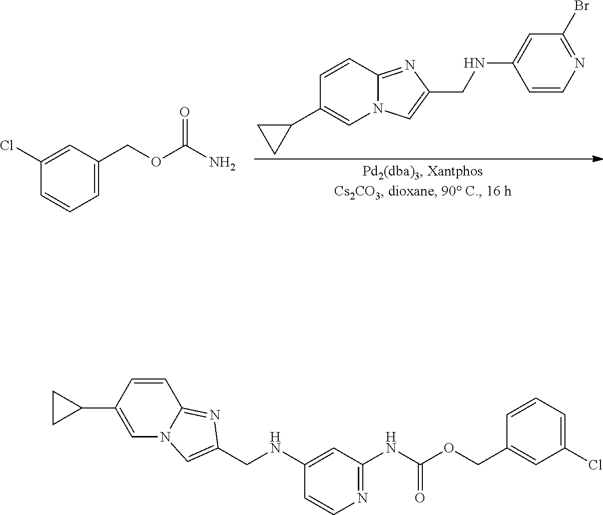 [SMILES: NC(=O)OCc1cccc(Cl)c1 ; Brc1cc(NCc2cn3cc(C4CC4)ccc3n2)ccn1 ; O=C(Nc1cc(NCc2cn3cc(C4CC4)ccc3n2)ccn1)OCc1cccc(Cl)c1]

A mixture of 3-chlorobenzyl carbamate (65 mg, 0.35 mmol), 2-bromo-N-((6-cyclopropylimidazo[1,2-a]pyridin-2-yl)methyl)pyridin-4-amine (120 mg, 0.35 mmol), Pd₂(dba)₃ (32 mg, 0.035 mmol), xantphos (40 mg, 0.070 mmol), and Cs₂CO₃ (228 mg, 0.70 mmol) in 1,4-dioxane (5 mL) was degassed with N₂ and stirred at 90° C. for 16 h. The mixture was filtered through Celite® and the filter cake was washed with DCM/MeOH (10/1, 50 mL). The filtrate was concentrated in vacuo to give the crude, which was purified by Prep-TLC(eluent: DCM/MeOH=20/1) to afford 1-(3-chlorophenyl)ethyl (4-(((6-cyclopropylimidazo[1,2-a]pyridin-2-yl)methyl)amino)pyridin-2-yl)carbamate (20 mg, 13% yield) as a white solid. ESI-MS [M+H]+: 448.1, ¹H NMR (400 MHz, DMSO) δ 9.79 (s, 1H), 8.30 (s, 1H), 7.73 (d, J=5.8 Hz, 1H), 7.63 (s, 1H), 7.50 (s, 1H), 7.43-7.34 (m, 4H), 7.15-7.08 (m, 2H), 6.96 (dd, J=9.3, 1.7 Hz, 1H), 6.30 (dd, J=5.8, 2.0 Hz, 1H), 5.13 (s, 2H), 4.35 (d, J=5.8 Hz, 2H), 1.92-1.85 (m, 1H), 0.97-0.79 (m, 2H), 0.70-0.61 (m, 2H).

### Example 14

### Synthesis of 3-chlorobenzyl (3-((6-cyclopropylimidazo[1,2-a]pyridin-2-yl)methyl)-[1,2,4]triazolo[4,3-a]pyrazin-6-yl)carbamate (I-14)

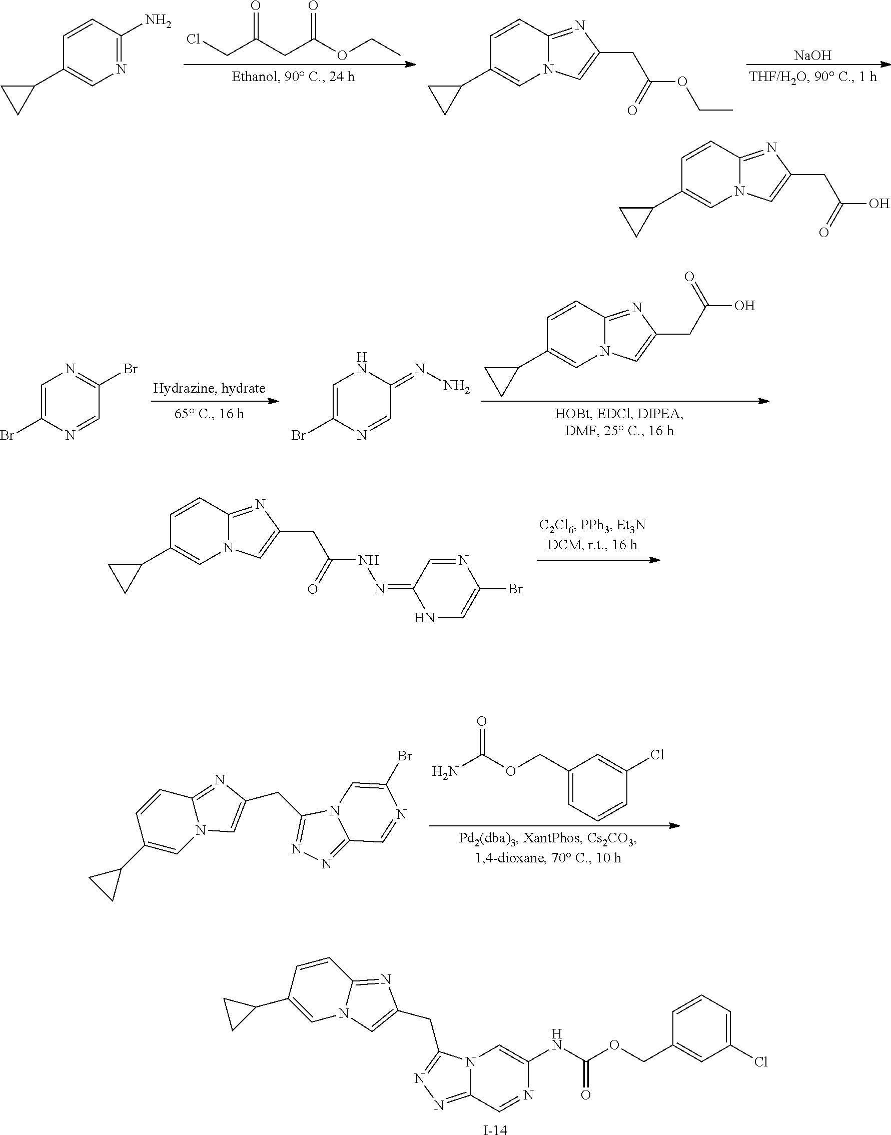

### Synthesis of ethyl 2-(6-cyclopropylimidazo[1,2-a]pyridin-2-yl)acetate

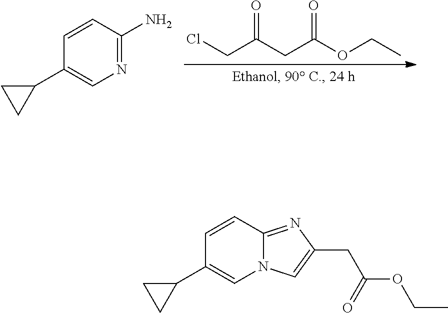

A mixture of ethyl 4-chloro-3-oxobutanoate (3.0 g, 18 mmol) and 5-cyclopropylpyridin-2-amine (2.0 g, 15 mmol) in ethanol (30 mL) was stirred at 90° C. for 24 h. Then the reaction mixture was concentrated in vacuo and the resulting residue was purified by column chromatography on silica gel (eluting with 20-60% EtOAc/PE) to afford ethyl 2-(6-cyclopropylimidazo[1,2-a]pyridin-2-yl)acetate (1.3 g, 36%) as a white solid. ESI-MS [M+H]+: 245.1

### Synthesis of 2-(6-cyclopropylimidazo[1,2-a]pyridin-2-yl)acetic acid

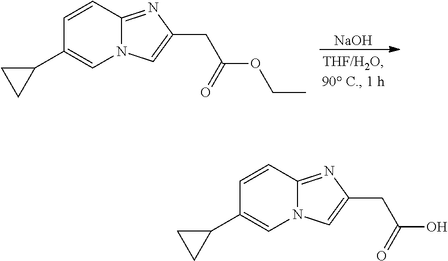 [SMILES: CCOC(=O)Cc1cn2cc(C3CC3)ccc2n1 ; "NaOH" ; O=C(O)Cc1cn2cc(C3CC3)ccc2n1]

To a solution of ethyl 2-(6-cyclopropylimidazo[1,2-a]pyridin-2-yl)acetate (1.3 g, 5.3 mmol) in THF (15 mL) and water (5 mL) was added NaOH (0.32 g, 8.0 mmol). This mixture was heated to 90° C. for 1 h. After cooling to room temperature, the resulting mixture was acidified with HCl (2 N in H₂O) to pH=6. The resulting precipitate was collected by filtration and then dried in vacuo to give 2-(6-cyclopropylimidazo[1,2-a]pyridin-2-yl)acetic acid (850 mg, 74%) as a brown solid. ESI-MS [M+H]+: 217.1.

### Synthesis of (E)-bromo-2-hydrazono-1,2-dihydropyrazine

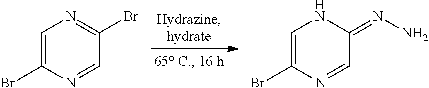 [SMILES: Brc1cnc(Br)cn1 ; N/N=c1\cnc(Br)c[nH]1]

A mixture of 2,5-dibromopyrazine (1.6 g, 6.7 mmol) and hydrazine hydrate (1.7 g, 34 mmol) in i-PrOH (20 mL) was stirred at 65° C. for 16 h. The reaction mixture was quenched with NaHCO₃ (sat. aq., 30 mL) and extracted with EtOAc (40 mL×5). The combined organics were washed with brine (20 mL), dried over Na₂SO₄ and concentrated in vacuo to give (E)-5-bromo-2-hydrazono-1,2-dihydropyrazine (1.1 g, crude) as a yellow solid. ESI-MS [M+H]+: 189.0.

### Synthesis of (E)-N′-(5-bromopyrazin-2(1H)-ylidene)-2-(6-cyclopropylimidazo[1,2-a]pyridin-2-yl)acetohydrazide

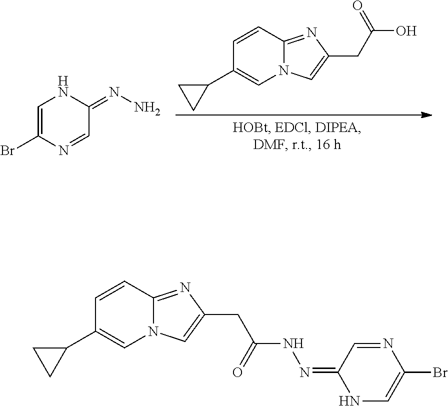 [SMILES: N/N=c1\cnc(Br)c[nH]1 ; O=C(O)Cc1cn2cc(C3CC3)ccc2n1 ; O=C(Cc1cn2cc(C3CC3)ccc2n1)N/N=c1\cnc(Br)c[nH]1]

A mixture of (E)-5-bromo-2-hydrazono-1,2-dihydropyrazine (400 mg, 2.1 mmol), 2-(6-cyclopropylimidazo[1,2-a]pyridin-2-yl)acetic acid (550 mg, 2.5 mmol), EDCI (1.2 g, 6.4 mmol), HOBt (0.86 g, 6.4 mmol), and DIPEA (1.6 g, 12 mmol) in DMF (10 mL) was stirred at room temperature for 16 h. The reaction mixture was quenched with water (60 mL) and extracted with EtOAc (50 mL×5). The combined organics were washed with brine (50 mL), dried over Na₂SO₄, then concentrated in vacuo to give the crude product, which was purified by column chromatography (eluent: DCM/MeOH=10/1) to give (E)-N′-(5-bromopyrazin-2(1H)-ylidene)-2-(6-cyclopropylimidazo[1,2-a]pyridin-2-yl)acetohydrazide (450 mg, 56%) as a yellow solid. ESI-MS [M+H]⁺: 387.0.

### Synthesis of 6-bromo-3-((6-cyclopropylimidazo[1,2-a]pyridin-2-yl)methyl)-[1,2,4]triazolo[4,3-a]pyrazine

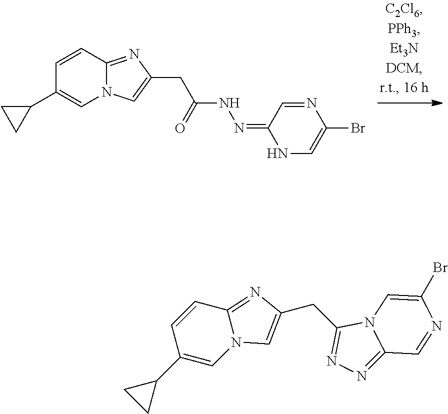 [SMILES: O=C(Cc1cn2cc(C3CC3)ccc2n1)N/N=c1\cnc(Br)c[nH]1 ; Brc1cn2c(Cc3cn4cc(C5CC5)ccc4n3)nnc2cn1]

To a suspension of (E)-N′-(5-bromopyrazin-2(1H)-ylidene)-2-(6-cyclopropylimidazo[1,2-a]pyridin-2-yl)acetohydrazide (290 mg, 0.75 mmol), C₂Cl₆ (0.71 g, 3.0 mmol), and PPh₃ (0.79 g, 3.0 mmol) in DCM (20 mL) was added Et₃N (0.30 g, 3.0 mmol) at room temperature, and the mixture was stirred at room temperature for 16 h under nitrogen. The reaction mixture was diluted with DCM (50 mL), washed with water (30 mL) and brine (30 mL), dried over Na₂SO₄, and concentrated in vacuo to give the crude product, which was purified by column chromatography (eluent: DCM/MeOH=0-10/1) to give 6-bromo-3-((6-cyclopropylimidazo[1,2-a]pyridin-2-yl)methyl)-[1,2,4]triazolo[4,3-a]pyrazine (190 mg, 69%) as a yellow solid. ESI-MS [M+H]+: 369.0.

### Synthesis of 3-chlorobenzyl (3-((6-cyclopropylimidazo[1,2-a]pyridin-2-yl)methyl)-[1,2,4]triazolo[4,3-a]pyrazin-6-yl)carbamate

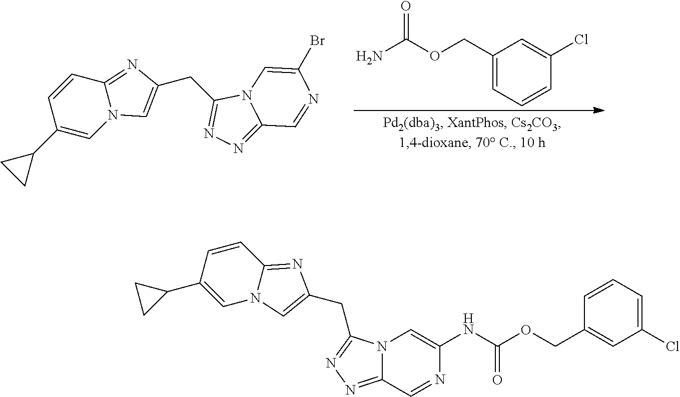 [SMILES: Brc1cn2c(Cc3cn4cc(C5CC5)ccc4n3)nnc2cn1 ; NC(=O)OCc1cccc(Cl)c1 ; O=C(Nc1cn2c(Cc3cn4cc(C5CC5)ccc4n3)nnc2cn1)OCc1cccc(Cl)c1]

A mixture of 6-bromo-3-((6-cyclopropylimidazo[1,2-a]pyridin-2-yl)methyl)-[1,2,4]triazolo[4,3-a]pyrazine (60 mg, 0.16 mmol), 3-chlorobenzyl carbamate (30 mg, 0.25 mmol), Pd₂(dba)₃ (15 mg, 0.016 mmol), Xantphos (19 mg, 0.033 mmol), and Cs₂CO₃ (0.16 g, 0.49 mmol) in 1,4-dioxane (8 mL) was stirred at 70° C. for 10 h under N₂. The reaction mixture was filtered through Celite® and the filter cake was washed with DCM/MeOH (10/1, 100 mL). The filtrate was concentrated in vacuo to give the crude, which was purified by column chromatography (eluent: DCM/MeOH=10/1) and Prep-HPLC to give the desired product (2.5 mg, 3.3%) as a white solid. ESI-MS [M+H]+: 474.1, ¹H NMR (400 MHz, MeOD) δ 9.11 (d, J=1.4 Hz, 1H), 8.57 (s, 1H), 8.18 (s, 1H), 7.71 (s, 1H), 7.41 (s, 1H), 7.40-7.29 (m, 4H), 7.08 (d, J=9.2 Hz, 1H), 5.15 (s, 2H), 4.72 (s, 2H), 1.97-1.91 (m, 1H), 1.00-0.93 (m, 2H), 0.73-0.67 (m, 2H).

### Example 15

### Synthesis of 1-(3-chlorophenyl)ethyl (4-(((6-cyclopropylimidazo[1,2-a]pyridin-2-yl)methyl)amino)pyridin-2-yl)carbamate (I-15)

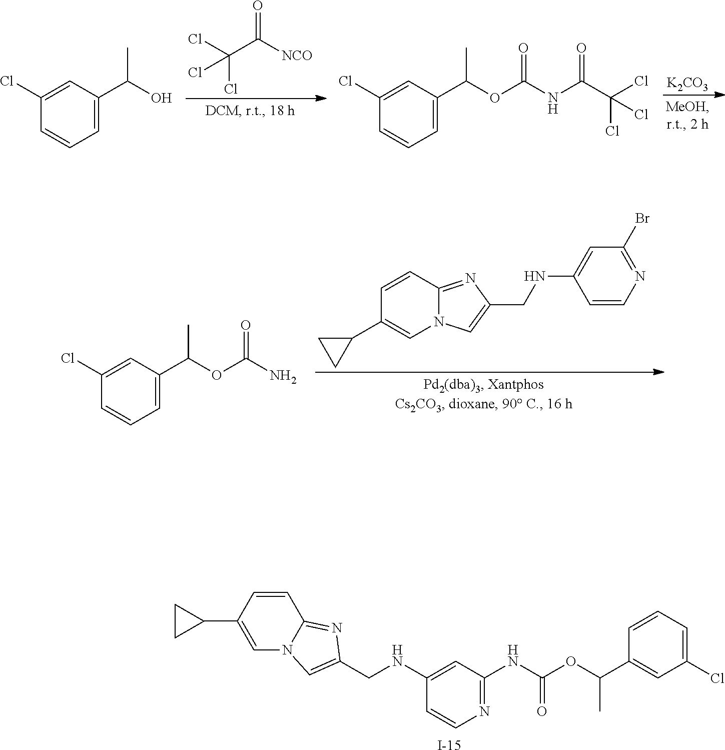 [0312] Synthesis of 1-(3-chlorophenyl)ethyl (2,2,2-trichloroacetyl)carbamate

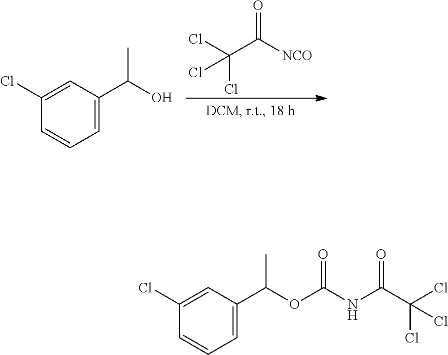

To a solution of 1-(3-chlorophenyl)ethan-1-ol (1.0 g, 6.4 mmol) in DCM (10 mL) was added 2,2,2-trichloroacetyl isocyanate (1.37 g, 7.3 mmol) at room temperature, and the mixture was stirred at room temperature for 18 h. The mixture was quenched with water (50 mL) and extracted with EtOAc (30 mL×3). The combined organic layers were washed with brine (30 mL), dried over Na₂SO₄, and concentrated in vacuo to afford 1-(3-chlorophenyl)ethyl (2,2,2-trichloroacetyl)carbamate (2 g, crude) as white solid, which was used in the next step directly. ESI-MS [M+H]+: 343.9.

### Synthesis of 1-(3-chlorophenyl)ethyl carbamate

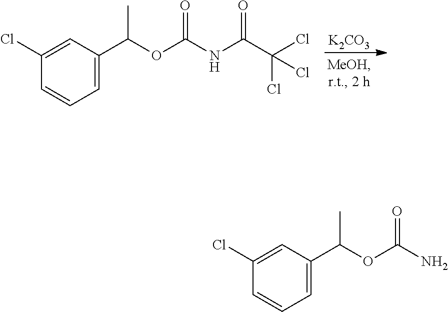

A mixture of 1-(3-chlorophenyl)ethyl (2,2,2-trichloroacetyl)carbamate (1.0 g, crude) and K₂CO₃ (1.2 g, 8.7 mmol) in MeOH (10 mL) was stirred at room temperature for 2 h. The mixture was quenched with water (50 mL) and extracted with EtOAc (30 mL×3). The combined organic layers were washed with brine (30 mL), dried over Na₂SO₄, and concentrated in vacuo. The residue was purified by flash column chromatography (eluent: EtOAc/PE from 0 to 30%) to give 1-(3-chlorophenyl)ethyl carbamate (500 mg, 78% yield) as white solid. ESI-MS [M+H]+: 200.0

### Synthesis of 1-(3-chlorophenyl)ethyl (4-(((6-cyclopropylimidazo[1,2-a]pyridin-2-yl)methyl)amino)pyridin-2-yl)carbamate

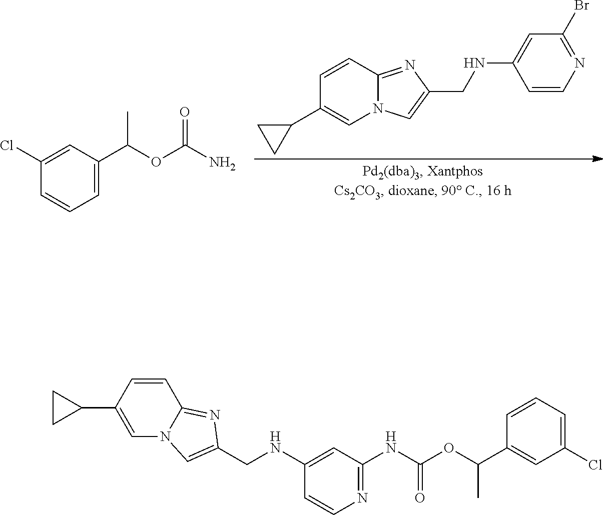 [SMILES: CC(OC(N)=O)c1cccc(Cl)c1 ; Brc1cc(NCc2cn3cc(C4CC4)ccc3n2)ccn1 ; CC(OC(=O)Nc1cc(NCc2cn3cc(C4CC4)ccc3n2)ccn1)c1cccc(Cl)c1]

A mixture of 1-(3-chlorophenyl)ethyl carbamate (70 mg, 0.35 mmol), 2-bromo-N-((6-cyclopropylimidazo[1,2-a]pyridin-2-yl)methyl)pyridin-4-amine (120 mg, 0.35 mmol), Pd₂(dba)₃ (32 mg, 0.035 mmol), xantphos (40 mg, 0.070 mmol), and Cs₂CO₃ (228 mg, 0.70 mmol) in 1,4-dioxane (5 mL) was degassed with N₂ and stirred at 90° C. for 16 h. The mixture was filtered through Celite® and the filter cake was washed with DCM/MeOH (10/1, 50 mL). The filtrate was concentrated in vacuo to give the crude, which was purified by column chromatography (eluent: DCM/MeOH=0˜5%) to afford 1-(3-chlorophenyl)ethyl (4-(((6-cyclopropylimidazo[1,2-a]pyridin-2-yl)methyl)amino)pyridin-2-yl)carbamate (15 mg, 9% yield) as a white solid. ESI-MS [M+H]+: 462.2, ¹H NMR (400 MHz, DMSO) δ 9.73 (s, 1H), 8.30-8.27 (m, 1H), 7.72 (d, J=5.8 Hz, 1H), 7.61 (s, 1H), 7.51-7.49 (m, 1H), 7.43-7.32 (m, 4H), 7.10-7.03 (m, 2H), 6.98-6.93 (m, 1H), 6.31-6.27 (m, 1H), 5.80-5.73 (m, 1H), 4.33 (d, J=5.8 Hz, 2H), 1.94-1.85 (m, 1H), 1.48 (d, J=6.6 Hz, 3H), 0.93-0.87 (m, 2H), 0.68-0.63 (m, 2H).

### Example 16

### Synthesis of 3-chlorobenzyl (1-((6-cyclopropylimidazo[1,2-a]pyridin-2-yl)methyl)-1H-imidazo[4,5-c]pyridin-6-yl)carbamate (I-16)

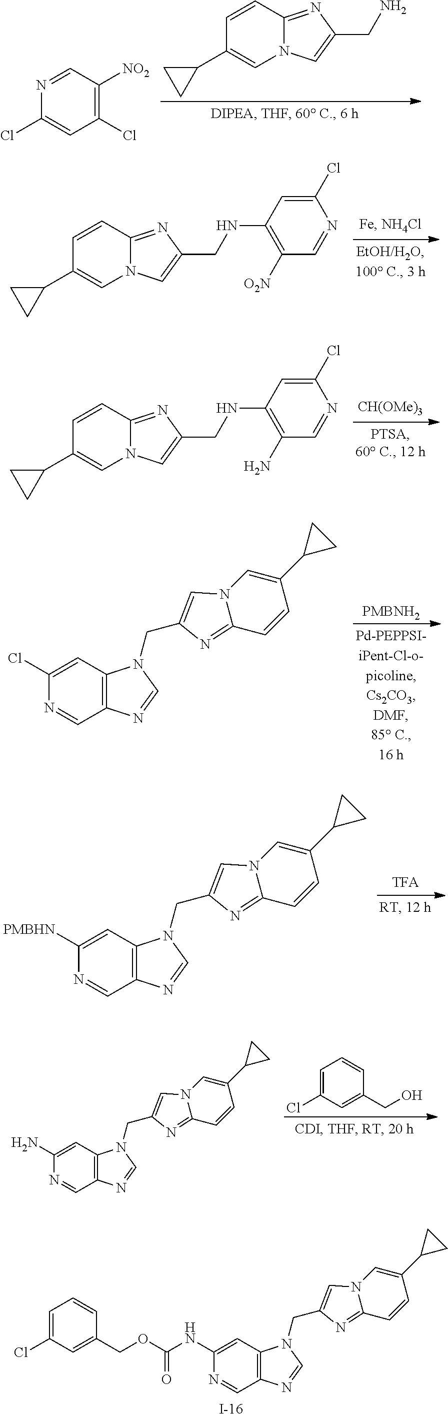

### Synthesis of 2-chloro-N-((6-cyclopropylimidazo[1,2-a]pyridin-2-yl)methyl)-5-nitropyridin-4-amine

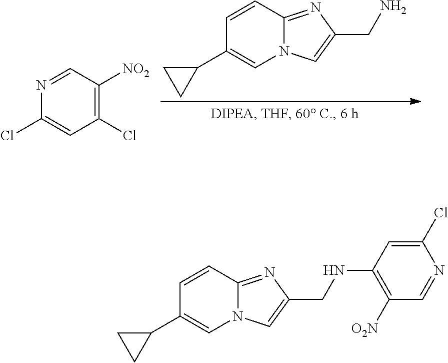

A mixture of 2,4-dichloro-5-nitropyridine (1 g, 5.18 mmol), (6-cyclopropylimidazo[1,2-a]pyridin-2-yl)methanamine (1.1 g, 6.22 mmol), and DIPEA (2 g, 15.54 mmol) in THF (20 mL) was stirred at 60° C. for 6 h. The mixture was quenched with NaHCO₃ (sat. aq., 50 mL) and extracted with EtOAc (50 mL×3). The combined organic layers were washed with brine (50 mL), dried over Na₂SO₄, and concentrated in vacuo. The residue was purified by column chromatography (eluent: DCM/MeOH=0-6%) to give 2-chloro-N-((6-cyclopropylimidazo[1,2-a]pyridin-2-yl)methyl)-5-nitropyridin-4-amine (1.5 g, 84%) as a white solid. ESI-MS [M+H]+: 344.2.

### Synthesis of 6-chloro-N′-((6-cyclopropylimidazo[1,2-a]pyridin-2-yl)methyl)pyridine-3,4-diamine

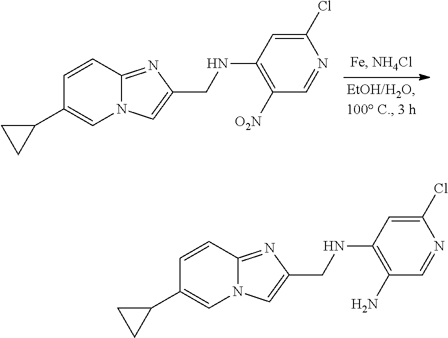 [SMILES: O=[N+]([O-])c1cnc(Cl)cc1NCc1cn2cc(C3CC3)ccc2n1 ; Nc1cnc(Cl)cc1NCc1cn2cc(C3CC3)ccc2n1]

To a mixture of 2-chloro-N-((6-cyclopropylimidazo[1,2-a]pyridin-2-yl)methyl)-5-nitropyridin-4-amine (1.5 g, 4.37 mmol) and NH₄Cl (2.3 g, 43.7 mmol) in EtOH/water (20 ml/5 mL) was added Fe (2.4 g, 43.7 mmol). The resulting reaction mixture was stirred at 100° C. for 3 h under N₂. After cooling to room temperature, the mixture was quenched with water (50 mL) and extracted with DCM (50 mL×5). The combined organic layers were washed with brine (50 mL), dried over Na₂SO₄, and concentrated in vacuo. The residue was purified by column chromatography (eluent: DCM/MeOH=0˜ 5%) to give 6-chloro-N⁴-((6-cyclopropylimidazo[1,2-a]pyridin-2-yl)methyl)pyridine-3,4-diamine (1.2 g, 88%) as a yellow solid. ESI-MS [M+H]+: 314.2.

### Synthesis of 5-(2,2-difluorocyclopropyl)-N,N-bis(4-methoxybenzyl)pyridin-2-amine

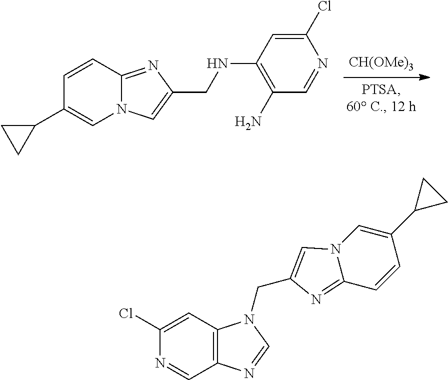

A mixture of 6-chloro-N⁴-((6-cyclopropylimidazo[1,2-a]pyridin-2-yl)methyl)pyridine-3,4-diamine (1.2 g, 3.83 mmol) and PTSA (65.0 mg, 0.38 mmol) in trimethoxymethane (5 ml) was stirred at 60° C. for 12 h. Water (50 mL) was added and the mixture was extracted with DCM (50 mL×3). The combined organic layers were washed with brine (50 mL), dried over Na₂SO₄, and concentrated in vacuo to give the crude, which was purified by column chromatography (eluent: PE/EtOAc=20/1˜ 2/1) to give 6-chloro-1-((6-cyclopropylimidazo[1,2-a]pyridin-2-yl)methyl)-1H-imidazo[4,5-c]pyridine (500 mg, 40%) as a yellow solid. ESI-MS [M+H]+: 324.2.

### Synthesis of 1-((6-cyclopropylimidazo[1,2-a]pyridin-2-yl)methyl)-N-(4-methoxybenzyl)-1H-imidazo [4,5-c]pyridin-6-amine

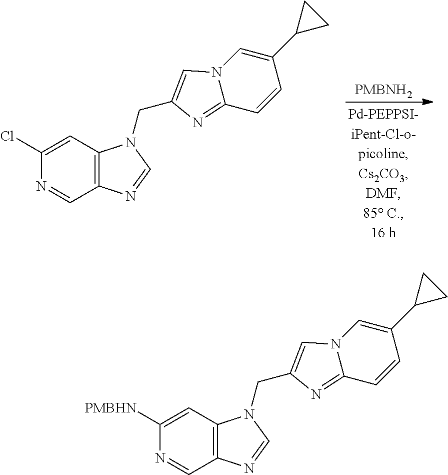

To a mixture of 6-chloro-1-((6-cyclopropylimidazo[1,2-a]pyridin-2-yl)methyl)-1H-imidazo[4,5-c]pyridine (500 mg, 1.55 mmol), PMBNH₂ (318.5 mg, 2.32 mmol), and Cs₂CO₃ (1.5 g, 4.65 mmol) in DMF (10 mL) was added Pd-PEPPSI-iPent-Cl-o-picoline (134 mg, 0.16 mmol). The reaction mixture was stirred at 85° C. for 16 h under N₂. Water (100 mL) was added, and the mixture was extracted with EtOAc (100 mL×3). The combined organic layers were washed with brine (50 mL), dried over Na₂SO₄ and concentrated in vacuo to give the crude, which was purified by column chromatography (eluent: PE/EtOAc=10/1˜ 2/1) to give 6-chloro-1-((6-cyclopropylimidazo[1,2-a]pyridin-2-yl)methyl)-1H-imidazo[4,5-c]pyridine (300 mg, 46%) as a yellow solid. ESI-MS [M+H]+: 425.2.

### Synthesis of 1-((6-cyclopropylimidazo[1,2-a]pyridin-2-yl)methyl)-1H-imidazo[4,5-c]pyridin-6-amine

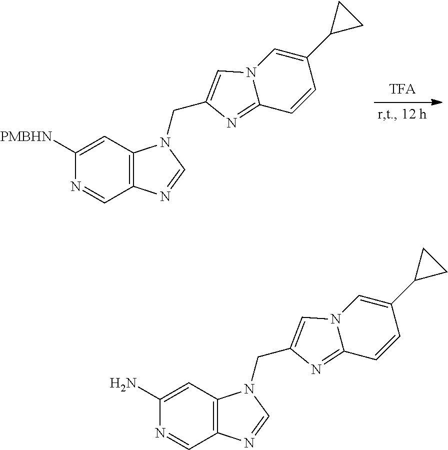

A solution of 6-chloro-1-((6-cyclopropylimidazo[1,2-a]pyridin-2-yl)methyl)-1H-imidazo[4,5-c]pyridine (300 mg, 0.71 mmol) in TFA (4 mL) was stirred at room temperature for 12 h. The mixture was concentrated in vacuo, and the residue was diluted with NaHCO₃ (sat. aq., 50 mL) and extracted with DCM (50 mL×3). The combined organic layers were washed with brine (30 mL), dried over Na₂SO₄, and concentrated in vacuo. The residue was purified by Prep-TLC (DCM/MeOH=20/1) to give 1-((6-cyclopropylimidazo[1,2-a]pyridin-2-yl)methyl)-1H-imidazo[4,5-c]pyridin-6-amine (140 mg, 65%) as a white solid. ESI-MS [M+H]+: 305.2.

### Synthesis of 3-chlorobenzyl (1-((6-cyclopropylimidazo[1,2-a]pyridin-2-yl)methyl)-1H-imidazo[4,5-c]pyridin-6-yl)carbamate

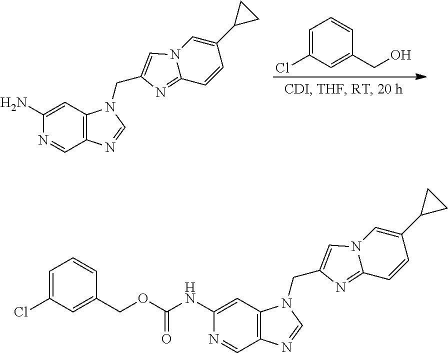 [SMILES: Nc1cc2c(cn1)ncn2Cc1cn2cc(C3CC3)ccc2n1 ; OCc1cccc(Cl)c1 ; O=C(Nc1cc2c(cn1)ncn2Cc1cn2cc(C3CC3)ccc2n1)OCc1cccc(Cl)c1]

To a mixture of CDI (29 mg, 0.18 mmol) in THF (3 mL) was added 1-((6-cyclopropylimidazo [1,2-a]pyridin-2-yl)methyl)-1H-imidazo[4,5-c]pyridin-6-amine (50 mg, 0.16 mmol) at 0° C., and the mixture was stirred at room temperature for 2 h. Then (3-chlorophenyl)methanol (28 mg, 0.20 mmol) was added thereto and the mixture was stirred at room temperature for another 18 h. The reaction was quenched with water (30 mL) and extracted with EtOAc (30 mL×3). The organic layers were washed with brine (30 mL), dried over Na₂SO₄, and concentrated in vacuo. The residue was purified by column chromatography (MeOH/DCM from 0 to 5%) to give 3-chlorobenzyl (1-((6-cyclopropylimidazo[1,2-a]pyridin-2-yl)methyl)-1H-imidazo[4,5-c]pyridin-6-yl)carbamate (22 mg, 29%) as a white solid. ESI-MS [M+H]+: 473.1, ¹H NMR (400 MHz, DMSO) δ 10.24 (s, 1H), 8.64 (d, J=0.9 Hz, 1H), 8.39 (s, 1H), 8.32 (s, 1H), 7.97 (d, J=0.9 Hz, 1H), 7.73 (s, 1H), 7.51 (s, 1H), 7.43-7.35 (m, 4H), 7.01-6.96 (m, 1H), 5.54 (s, 2H), 5.17 (s, 2H), 1.96-1.85 (m, 1H), 0.95-0.85 (m, 2H), 0.70-0.60 (m, 2H).

### Example 17

### Synthesis of 1-(3-chlorophenyl)ethyl (1-((6-cyclopropylimidazo[1,2-a]pyridin-2-yl)methyl)-1H-imidazo[4,5-c]pyridin-6-yl)carbamate (I-17)

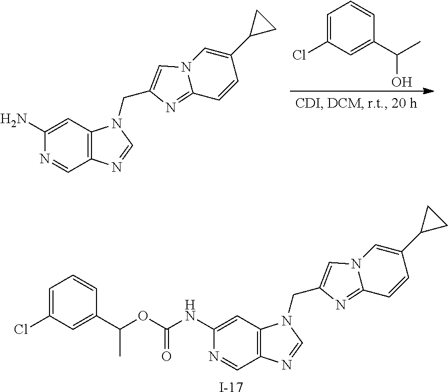

Synthesis of 1-(3-chlorophenyl)ethyl (1-((6-cyclopropylimidazo[1,2-a]pyridin-2-yl)methyl)-1H-imidazo[4,5-c]pyridin-6-yl)carbamate. To a mixture of CDI (29 mg, 0.18 mmol) in THF (3 mL) was added 1-((6-cyclopropylimidazo [1,2-a]pyridin-2-yl)methyl)-1H-imidazo[4,5-c]pyridin-6-amine (50 mg, 0.16 mmol) at 0° C., and the resulting mixture was stirred at room temperature for 2 h. Then 1-(3-chlorophenyl)ethan-1-ol (31 mg, 0.20 mmol) was added and the mixture was stirred at room temperature for another 18 h. The reaction was quenched with water (30 mL) and extracted with EtOAc (30 mL×3). The organic layers were washed with brine (30 mL), dried over Na₂SO₄, and concentrated in vacuo. The residue was purified by column chromatography (MeOH/DCM from 0 to 5%) to give 1-(3-chlorophenyl)ethyl (1-((6-cyclopropylimidazo[1,2-a]pyridin-2-yl)methyl)-1H-imidazo[4,5-c]pyridin-6-yl)carbamate (35 mg, 45%) as a white solid. ESI-MS [M+H]+: 487.2. ¹H NMR (400 MHz, DMSO) δ 10.19 (s, 1H), 8.64 (d, J=0.9 Hz, 1H), 8.38 (s, 1H), 8.31 (s, 1H), 7.93 (d, J=0.9 Hz, 1H), 7.70 (s, 1H), 7.52 (s, 1H), 7.43-7.32 (m, 4H), 7.00-6.95 (m, 1H), 5.83-5.78 (m, 1H), 5.52 (s, 2H), 1.95-1.84 (m, 1H), 1.51 (d, J=6.6 Hz, 3H), 0.92-0.87 (m, 2H), 0.67-0.62 (m, 2H).

### Examples 18 and 19

### Synthesis of 3-chlorobenzyl (1-((6-cyclopropylimidazo[1,2-a]pyridin-2-yl)methyl)-1H-pyrazolo[4,3-c]pyridin-4-yl)carbamate (I-18) and 3-chlorobenzyl (2-((6-cyclopropylimidazo[1,2-a]pyridin-2-yl)methyl)-1H-pyrazolo[4,3-c]pyridin-4-yl)carbamate (1-19)

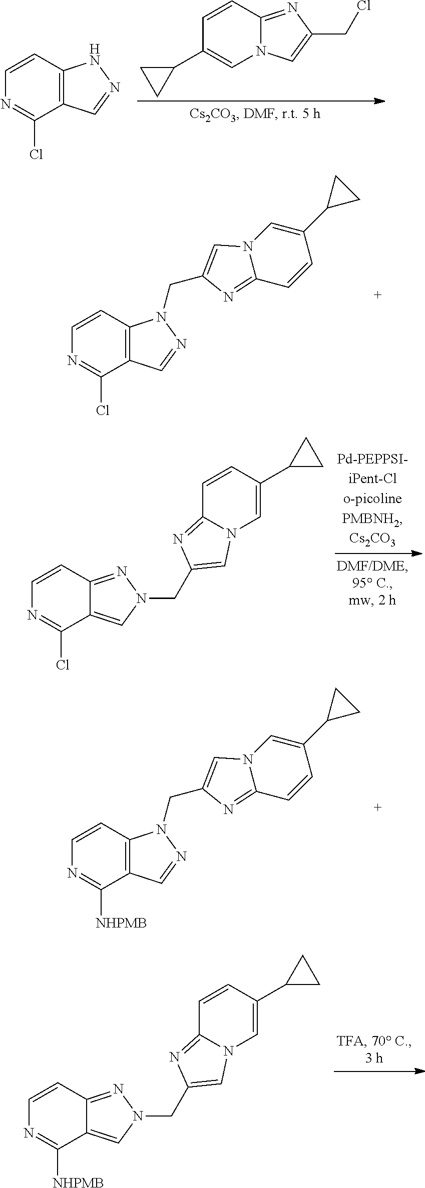

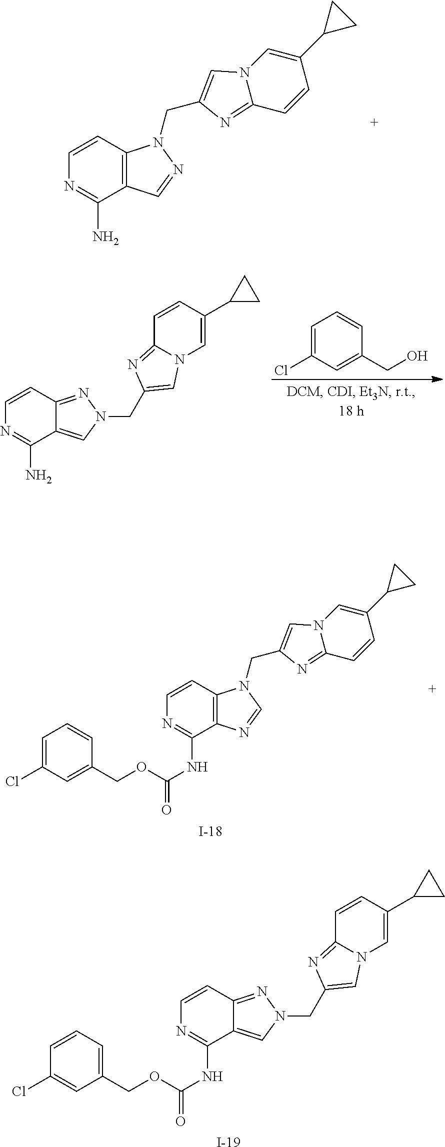

### Synthesis of 4-chloro-1-((6-cyclopropylimidazo[1,2-a]pyridin-2-yl)methyl)-1H-pyrazolo[4,3-c]pyridine

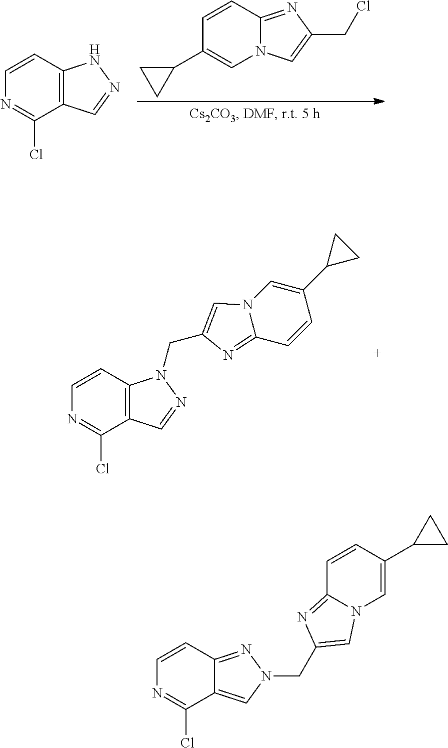

To a solution of 2-(chloromethyl)-6-cyclopropylimidazo[1,2-a]pyridine (673 mg, 3.26 mmol) in DMF(30 mL) was added 4-chloro-1H-pyrazolo[4,3-c]pyridine (500 mg, 3.26 mmol) and Cs₂CO₃ (2.13 g, 6.52 mmol), then the mixture was stirred at room temperature for 5 h. Water (100 mL) was added, and the mixture was extracted with EtOAc (3×100 mL). The combined organic layers were washed with brine (100 mL), dried over anhydrous Na₂SO₄, and concentrated in vacuo to give the crude, which was purified by Prep-TLC (DCM/MeOH=50/1) to give 4-chloro-1-((6-cyclopropylimidazo[1,2-a]pyridin-2-yl)methyl)-1H-pyrazolo[4,3-c]pyridine and 4-chloro-2-((6-cyclopropylimidazo[1,2-a]pyridin-2-yl)methyl)-1H-pyrazolo[4,3-c]pyridine (900 mg, 85%, mixture) as a yellow solid. ESI-MS [M+H]+: 324.1.

### Synthesis of 1-((6-cyclopropylimidazo[1,2-a]pyridin-2-yl)methyl)-N-(4-methoxybenzyl)-1H-pyrazolo[4,3-c]pyridin-4-amine

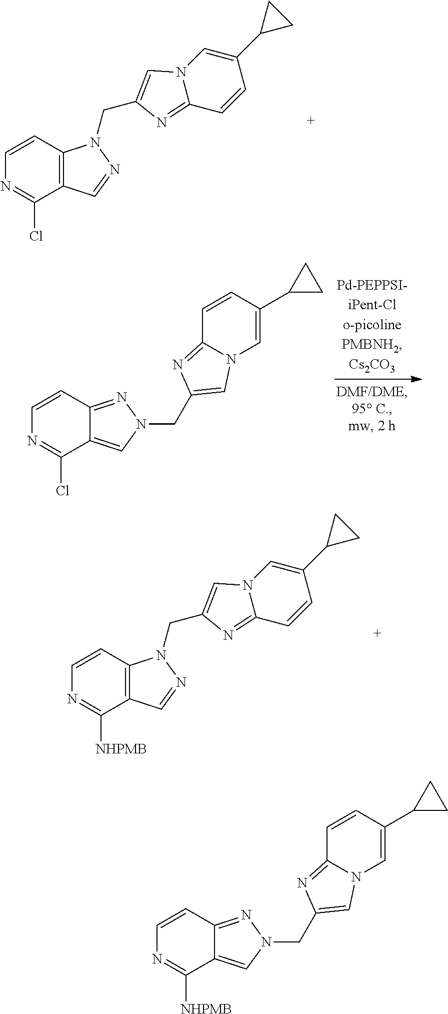

To a solution of 4-chloro-1-((6-cyclopropylimidazo[1,2-a]pyridin-2-yl)methyl)-1H-pyrazolo[4,3-c]pyridine (350 mg, 1.08 mmol, mixture) in DMF (1 mL) and DME (1.6 mL) was added (4-methoxyphenyl) methanamine (222 mg, 1.62 mmol), Pd-PEPPSI-iPent-Cl o-picoline (42 mg, 0.05 mmol), and Cs₂CO₃ (1.05 g, 3.24 mmol). After degassing with N₂ for 1 min, the reaction was irradiated in a microwave reactor at 95° C. for 2 h. The mixture was quenched with water (20 mL) and extracted with EtOAc (20 mL×4). The combined organic layers were washed with brine (20 mL), dried over Na₂SO₄, and concentrated in vacuo. The residue was purified by column chromatography (eluent: DCM:MeOH=0-10%) to give 1-((6-cyclopropylimidazo[1,2-a]pyridin-2-yl)methyl)-N-(4-methoxybenzyl)-1H-pyrazolo[4,3-c]pyridin-4-amine and 2-((6-cyclopropylimidazo[1,2-a]pyridin-2-yl)methyl)-N-(4-methoxybenzyl)-2H-pyrazolo[4,3-c]pyridin-4-amine (300 mg, 66%, mixture) as a yellow oil. ESI-MS [M+H]⁺: 425.2.

### Synthesis of 1-((6-cyclopropylimidazo[1,2-a]pyridin-2-yl)methyl)-1H-pyrazolo[4,3-c]pyridin-4-amine

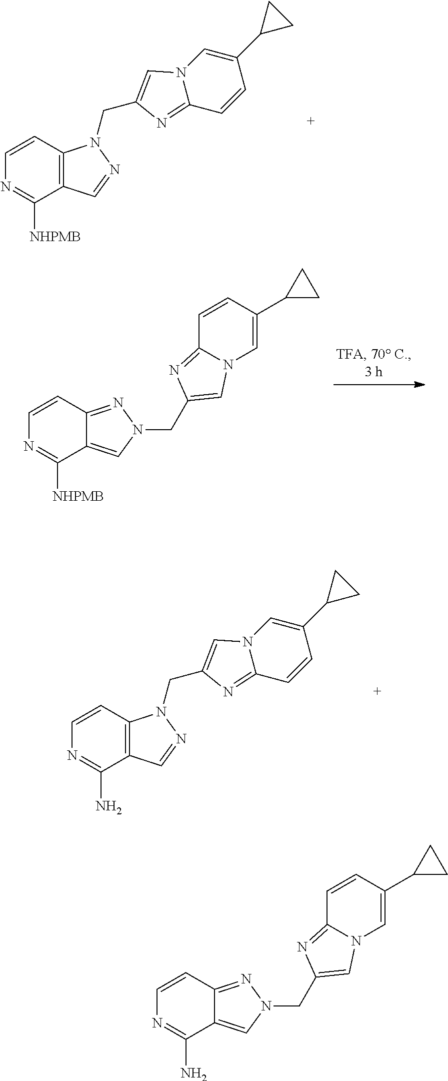

A solution of 1-((6-cyclopropylimidazo[1,2-a]pyridin-2-yl)methyl)-N-(4-methoxybenzyl)-1H-pyrazolo[4,3-c]pyridin-4-amine (300 mg, 0.71 mmol, mixture) in trifluoroacetic acid (10 mL) was stirred at 70° C. for 3 h. The mixture was quenched with NaHCO₃ (sat. aq., 100 mL) and extracted with EtOAc (30 mL×4). The combined organic layers were washed with brine (20 mL), dried over Na₂SO₄, and concentrated in vacuo to give 1-((6-cyclopropylimidazo[1,2-a]pyridin-2-yl)methyl)-1H-pyrazolo[4,3-c]pyridin-4-amine and 2-((6-cyclopropylimidazo[1,2-a]pyridin-2-yl)methyl)-1H-pyrazolo[4,3-c]pyridin-4-amine (180 mg, 83% yield, mixture) as a yellow oil which was used without purification. ESI-MS [M+H]⁺: 305.1.

### Synthesis of 3-chlorobenzyl (1-((6-cyclopropylimidazo[1,2-a]pyridin-2-yl)methyl)-1H-pyrazolo[4,3-c]pyridin-4-yl)carbamate

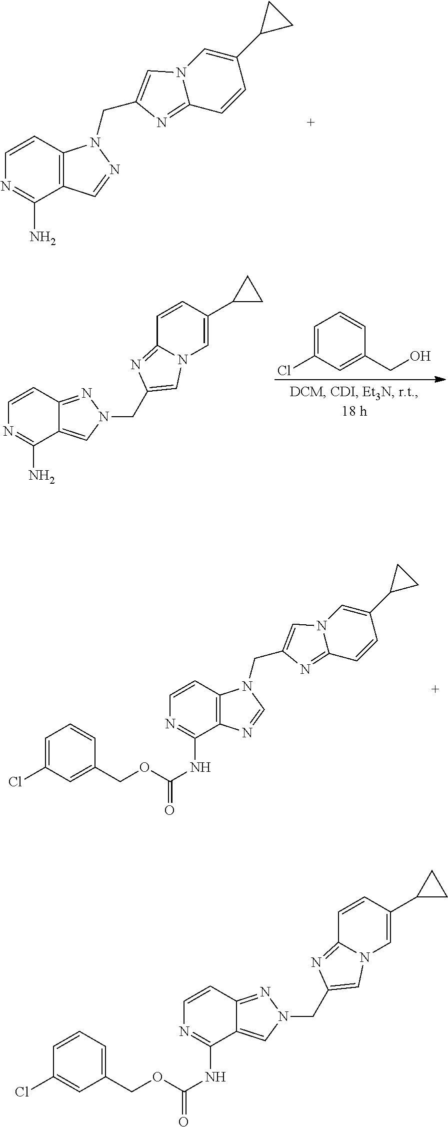 [SMILES: Nc1nccc2c1cnn2Cc1cn2cc(C3CC3)ccc2n1 ; Nc1nccc2nn(Cc3cn4cc(C5CC5)ccc4n3)cc12 ; OCc1cccc(Cl)c1 ; O=C(Nc1nccc2c1ncn2Cc1cn2cc(C3CC3)ccc2n1)OCc1cccc(Cl)c1 ; O=C(Nc1nccc2nn(Cc3cn4cc(C5CC5)ccc4n3)cc12)OCc1cccc(Cl)c1]

To a mixture of 1-((6-cyclopropylimidazo[1,2-a]pyridin-2-yl)methyl)-1H-pyrazolo[4,3-c]pyridin-4-amine (180 mg, 0.71 mmol, mixture) in DCM (8 mL) was added CDI (170 mg, 1.05 mmol) and Et₃N (266 mg, 2.63 mmol) at room temperature, and the mixture was stirred at room temperature for 2 h. (3-chlorophenyl) methanol (150 mg, 1.05 mmol) was added and the mixture was stirred at room temperature for 16 h. The reaction was quenched with water (30 mL) then extracted with EtOAc (30 mL×3). The organic layers were washed with brine (30 mL), dried over Na₂SO₄, and concentrated in vacuo. The residue was purified by Prep-TLC (DCM:MeOH=25:1) to give 3-chlorobenzyl (1-((6-cyclopropylimidazo[1,2-a]pyridin-2-yl)methyl)-1H-pyrazolo[4,3-c]pyridin-4-yl)carbamate (62.8 mg, 19% yield) as a white solid AND 3-chlorobenzyl (2-((6-cyclopropylimidazo[1,2-a]pyridin-2-yl)methyl)-2H-pyrazolo[4,3-c]pyridin-4-yl)carbamate (16.7 mg, 6%) as a white solid.

3-chlorobenzyl (1-((6-cyclopropylimidazo[1,2-a]pyridin-2-yl)methyl)-1H-pyrazolo[4,3-c]pyridin-4-yl)carbamate: ESI-MS [M+H]+: 473.1, ¹H NMR (400 MHz, DMSO) δ 10.54 (s, 1H), 8.28 (s, 1H), 8.25 (s, 1H), 8.04-7.97 (m, 1H), 7.62 (s, 1H), 7.53 (s, 1H), 7.49-7.43 (m, 1H), 7.41-7.37 (m, 3H), 7.32 (d, J=9.3 Hz, 1H), 6.97-6.90 (m, 1H), 5.67 (s, 2H), 5.20 (s, 2H), 1.90-1.83 (m, 1H), 0.89-0.84 (m, 2H), 0.63-0.59 (m, 2H).
3-chlorobenzyl (2-((6-cyclopropylimidazo[1,2-a]pyridin-2-yl)methyl)-2H-pyrazolo[4,3-c]pyridin-4-yl)carbamate:
ESI-MS [M+H]⁺: 473.1. ¹H NMR (400 MHz, DMSO) δ 10.52 (s, 1H), 8.72 (s, 1H), 8.32 (s, 1H), 7.80 (s, 1H), 7.73-7.65 (m, 1H), 7.48 (s, 1H), 7.41-7.35 (m, 4H), 7.11-7.05 (m, 1H), 6.97 (dd, J=9.4, 1.7 Hz, 1H), 5.68 (s, 2H), 5.15 (s, 2H), 1.92-1.87 m, 1H), 0.90-0.86 (m, 2H), 0.65-0.61 (m, 2H).

### Example 20

### Synthesis of 3-chlorobenzyl (1-((6-cyclopropylimidazo[1,2-a]pyridin-2-yl)methyl)-1H-[1,2,3]triazolo[4,5-c]pyridin-6-yl)carbamate (I-20)

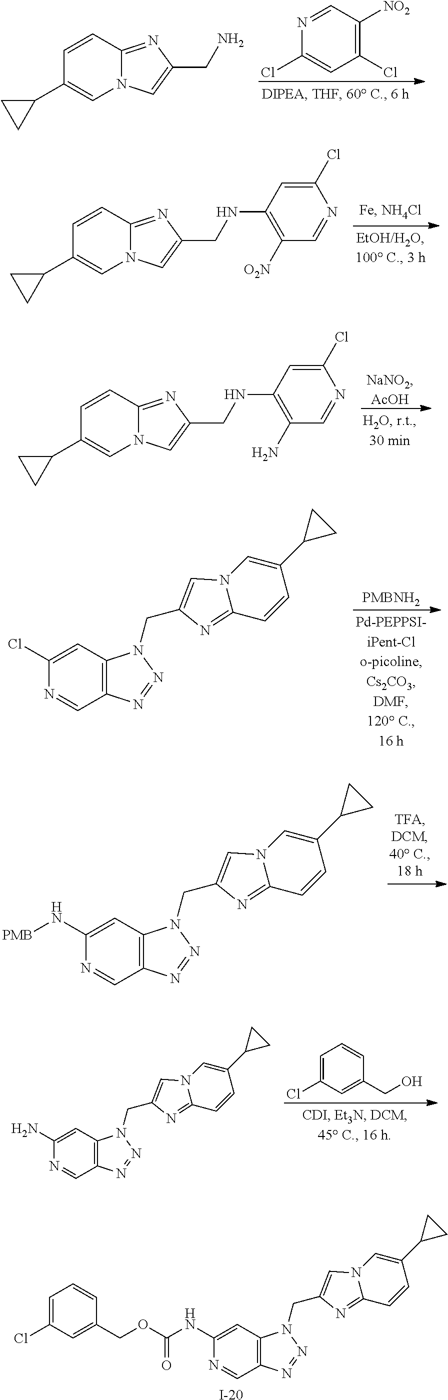

### Synthesis of 2-chloro-N-((6-cyclopropylimidazo[1,2-a]pyridin-2-yl)methyl)-5-nitropyridin-4-amine

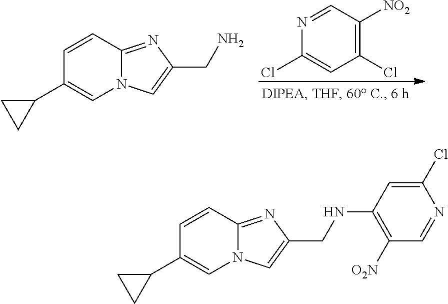 [SMILES: NCc1cn2cc(C3CC3)ccc2n1 ; O=[N+]([O-])c1cnc(Cl)cc1Cl ; O=[N+]([O-])c1cnc(Cl)cc1NCc1cn2cc(C3CC3)ccc2n1]

A mixture of 2,4-dichloro-5-nitropyridine (1 g, 5.18 mmol), (6-cyclopropylimidazo[1,2-a]pyridin-2-yl)methanamine (1.1 g, 6.22 mmol), and DIPEA (2 g, 15.54 mmol) in THF (20 mL) was stirred at 60° C. for 6 h. The mixture was quenched with NaHCO₃ (sat. aq., 50 mL) and extracted with EtOAc (50 mL×3). The combined organic layers were washed with brine (50 mL), dried over Na₂SO₄, and concentrated in vacuo. The residue was purified by column chromatography (eluent: DCM/MeOH=0-6%) to give 2-chloro-N-((6-cyclopropylimidazo[1,2-a]pyridin-2-yl)methyl)-5-nitropyridin-4-amine (1.5 g, 84%) as a white solid. ESI-MS [M+H]+: 344.2.

### Synthesis of 6-chloro-N⁴-((6-cyclopropylimidazo[1,2-a]pyridin-2-yl)methyl)pyridine-3,4-diamine

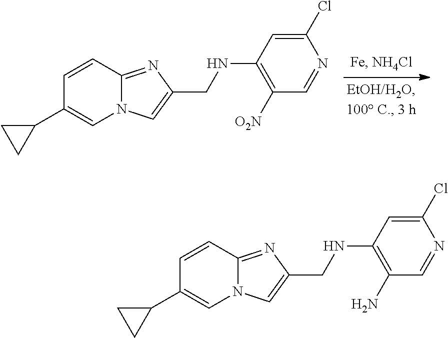 [SMILES: O=[N+]([O-])c1cnc(Cl)cc1NCc1cn2cc(C3CC3)ccc2n1 ; Nc1cnc(Cl)cc1NCc1cn2cc(C3CC3)ccc2n1]

To a mixture of 2-chloro-N-((6-cyclopropylimidazo[1,2-a]pyridin-2-yl)methyl)-5-nitropyridin-4-amine (1.5 g, 4.37 mmol) and NH₄Cl (2.3 g, 43.7 mmol) in EtOH/water (20 ml/5 mL) was added Fe (2.4 g, 43.7 mmol). The resulting reaction mixture was stirred at 100° C. for 3 h under N₂. After cooling to room temperature, the mixture was quenched with water (50 mL) and extracted with DCM (50 mL×5). The combined organic layers were washed with brine (50 mL), dried over Na₂SO₄ and concentrated. The residue was purified by column chromatography (eluent: DCM/MeOH=0˜ 5%) to give 6-chloro-N⁴-((6-cyclopropylimidazo[1,2-a]pyridin-2-yl)methyl)pyridine-3,4-diamine (1.2 g, 88%) as a yellow solid. ESI-MS [M+H]⁺: 314.2.

### Synthesis of 6-chloro-1-((6-cyclopropylimidazo[1,2-a]pyridin-2-yl)methyl)-1H-[1,2,3]triazolo[4,5-c]pyridine

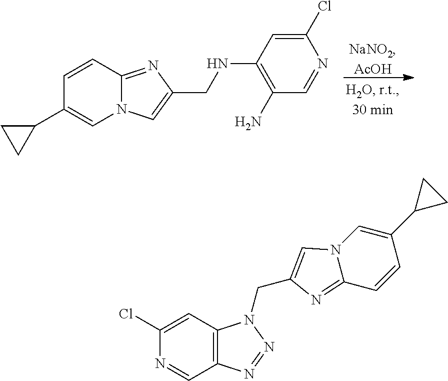

To a solution of of 6-chloro-N⁴-((6-cyclopropylimidazo[1,2-a]pyridin-2-yl)methyl)pyridine-3,4-diamine (500 mg, 1.59 mmol) in AcOH(15 ml) was added a solution of NaNO₂(165 mg, 2.38 mmol) in H₂O (15 ml) at room temperature. The resulting solution was stirred at this temperature for 30 min, then extracted with ethyl acetate (30 ml×3). The combined organic layers were evaporated in vacuo, and the residue was purified by chromatography on silica gel (MeOH:DCM=1:20) to give 6-chloro-1-((6-cyclopropylimidazo[1,2-a]pyridin-2-yl)methyl)-1H-[1,2,3]triazolo[4,5-c]pyridine (400 mg, 77%) as a yellow solid. ESI-MS [M+H]⁺: 325.1.

### Synthesis of 1-((6-cyclopropylimidazo[1,2-a]pyridin-2-yl)methyl)-N-(4-methoxybenzyl)-1H-[1-1,2,3]triazolo[4,5-c]pyridin-6-amine

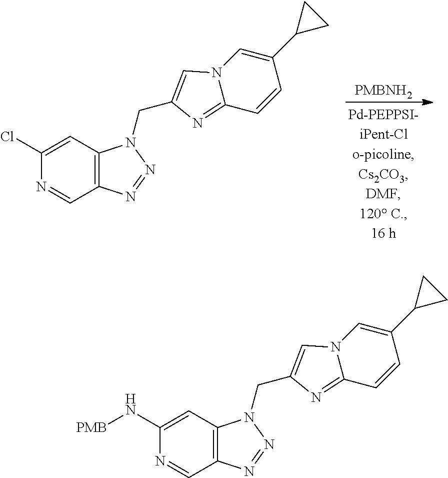

To a mixture of 6-chloro-1-((6-cyclopropylimidazo[1,2-a]pyridin-2-yl)methyl)-1H-[1,2,3]triazolo[4,5-c]pyridine (400 mg, 1.23 mmol), PMBNH₂ (342 mg, 2. mmol), and Cs₂CO₃ (1.46 g, 4.5 mmol) in DMF (10 mL) was added Pd-PEPPSI-iPent-Cl-o-picoline (100 mg, 0.12 mmol). The reaction mixture was stirred at 85° C. for 16 h under N₂. Water (100 mL) was added, and the mixture was extracted with EtOAc (100 mL×3). The combined organic layers were washed with brine (50 mL), dried over Na₂SO₄, and concentrated in vacuo to give the crude, which was purified by column chromatography (eluent: PE/EtOAc=10/1˜ 2/1) to give 1-((6-cyclopropylimidazo[1,2-a]pyridin-2-yl)methyl)-N-(4-methoxybenzyl)-1H-[1,2,3]triazolo[4,5-c]pyridin-6-amine (300 mg, 56%) as a yellow solid. ESI-MS [M+H]+: 426.2.

### Synthesis of 1-((6-cyclopropylimidazo[1,2-a]pyridin-2-yl)methyl)-1H-[1,2,3]triazolo[4,5-c]pyridin-6-amine

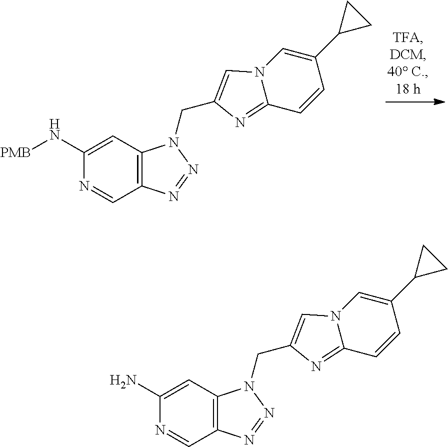

A solution of 1-((6-cyclopropylimidazo[1,2-a]pyridin-2-yl)methyl)-N-(4-methoxybenzyl)-1H-[1,2,3]triazolo[4,5-c]pyridin-6-amine (300 mg, 0.71 mmol) in TFA (4 mL) was stirred at room temperature for 12 h. The mixture was concentrated, and the residue was diluted with NaHCO₃ (sat. aq., 50 mL) and extracted with DCM (50 mL×3). The combined organic layers were washed with brine (30 mL), dried over Na₂SO₄, and concentrated in vacuo. The residue was purified by Prep-TLC (DCM/MeOH=20/1) to give 1-((6-cyclopropylimidazo[1,2-a]pyridin-2-yl)methyl)-1H-[1,2,3]triazolo[4,5-c]pyridin-6-amine (140 mg, 65%) as a white solid. ESI-MS [M+H]⁺: 306.2.

### Synthesis of 3-chlorobenzyl (1-((6-cyclopropylimidazo[1,2-a]pyridin-2-yl)methyl)-1H-[1,2,3]triazolo[4,5-c]pyridin-6-yl)carbamate

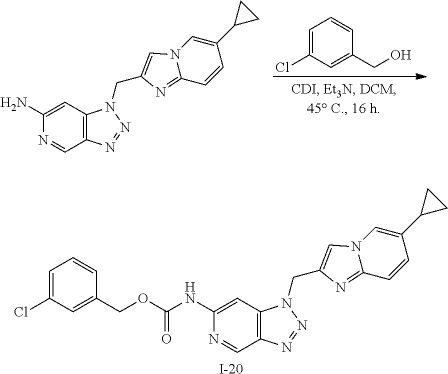

A mixture of 1-((6-cyclopropylimidazo[1,2-a]pyridin-2-yl)methyl)-1H-[1,2,3]triazolo[4,5-c]pyridin-6-amine (35 mg, 0.11 mmol), CDI (37 mg, 0.22 mmol), and Et₃N (364 mg, 3.6 mmol) in DCM (2 mL) was a stirred at 45° C. for 16 h. (3-chlorophenyl)methanol (196 mg, 1.38 mmol) was added and the mixture was stirred at 45° C. for another 2 h. The reaction was quenched with water (30 mL) and extracted with DCM (10 mL×3). The combined organic layers were washed with water (30 mL), dried over anhydrous Na₂SO₄, and concentrated to give the crude, which was purified by Prep-TLC (eluent: DCM/MeOH=10/1) to give 3-chlorobenzyl (1-((6-cyclopropylimidazo[1,2-a] pyridin-2-yl) methyl)-1H-[1,2,3] triazolo[4,5-c]pyridin-6-yl)carbamate (14.3 mg, 28%) as a white solid. ESI-MS [M+H]+: 474.1. ¹H NMR (400 MHz, DMSO) δ 10.64 (s, 1H), 9.17 (s, 1H), 8.31 (s, 1H), 8.08 (s, 1H), 7.80 (s, 1H), 7.50 (s, 1H), 7.42-7.31 (m, 4H), 6.95 (d, J=8.5 Hz, 1H), 5.98 (s, 2H), 5.17 (s, 2H), 1.91-1.85 (m, 1H), 0.88-0.85 (m, 2H), 0.65-0.61 (m, 2H).

### Example 21

### Synthesis of 1-(5-chloro-2-(1H-tetrazol-1-yl)phenyl)ethyl (4-(((6-cyclopropylimidazo[1,2-a]pyridin-2-yl)methyl)amino)pyridin-2-yl)carbamate (I-21)

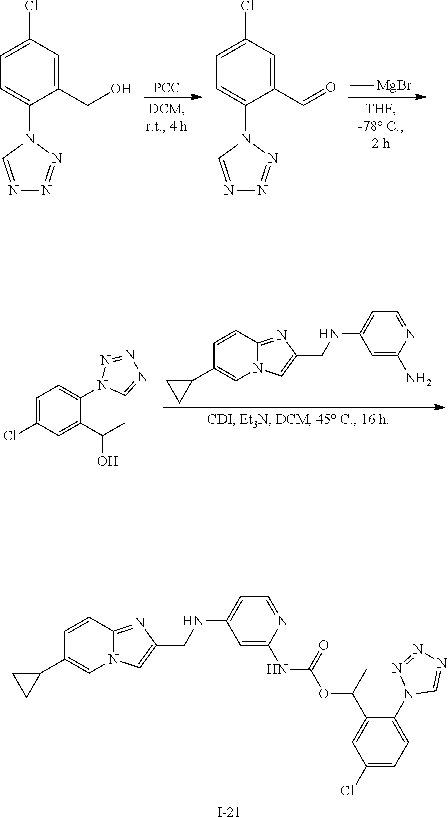

### Synthesis of 5-chloro-2-(1H-tetrazol-1-yl)benzaldehyde

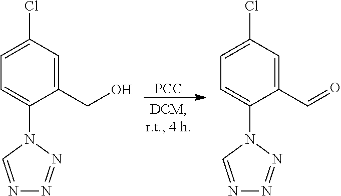

To a solution of (5-chloro-2-(1H-tetrazol-1-yl)phenyl)methanol (500 mg, 2.37 mmol) in DCM (20 mL) was added PCC (1.023 g, 4.75 mmol) at room temperature. The mixture was stirred at room temperature for 4 h. The reaction was concentrated to give the crude, which was purified by silica gel chromatography (eluent: DCM/MeOH=20/1) to give 5-chloro-2-(1H-tetrazol-1-yl)benzaldehyde (423 mg, 86%) as a yellow oil. ESI-MS [M+H]⁺: 209.0.

### Synthesis of 1-(5-chloro-2-(1H-tetrazol-1-yl)phenyl)ethan-1-ol

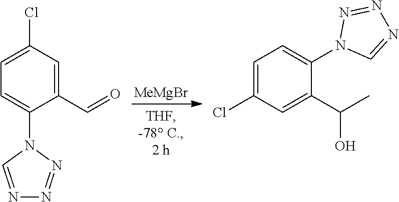

To a solution of 5-chloro-2-(1H-tetrazol-1-yl)benzaldehyde (100 mg, 0.48 mmol) in THF (0.3 mL) was added methylmagnesium bromide (3M in THF, 0.32 mL, 0.96 mmol,) at −78° C. under N₂. The mixture was stirred at −78° C. for 2 h. The reaction was quenched with saturated aqueous NH₄Cl (20 mL) and extracted with EtOAc (30 mL×3). The combined organic layers were washed with brine (30 mL), dried over anhydrous Na₂SO₄, and concentrated in vacuo to give the crude, which was purified by Prep-TLC (eluent: DCM/MeOH=20/1) to give 1-(5-chloro-2-(1H-tetrazol-1-yl)phenyl)ethan-1-ol (32 mg, 30%) as a white solid. ESI-MS [M+H]⁺: 225.0.

### Synthesis of 1-(5-chloro-2-(1H-tetrazol-1-yl)phenyl)ethyl (4-(((6-cyclopropylimidazo[1,2-a]pyridin-2-yl)methyl)amino)pyridin-2-yl)carbamate

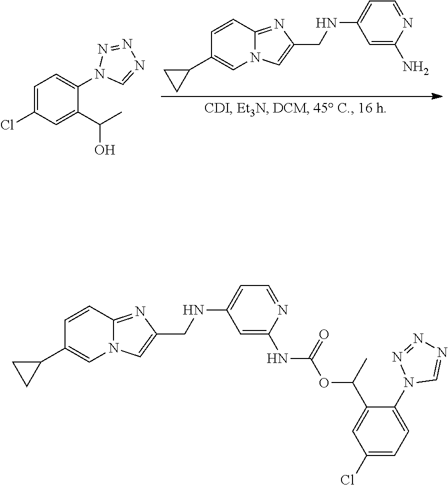

To a solution of N⁴-((6-cyclopropylimidazo[1,2-a]pyridin-2-yl)methyl)pyridine-2,4-diamine (20 mg, 0.071 mmol) in DCM (1.5 mL) was added CDI (23 mg, 0.142 mmol) and Et₃N (0.05 mL, 36 mg, 0.355 mmol) at room temperature. The mixture was stirred at 45° C. for 2 h. Then, 1-(5-chloro-2-(1H-tetrazol-1-yl)phenyl)ethan-1-ol (32 mg, 0.14 mmol) was added and the reaction mixture was stirred at 45° C. for 16 h. The reaction mixture was concentrated in vacuo to give the crude, which was purified by silica gel chromatography (eluent: DCM/MeOH=20/1) to give 1-(5-chloro-2-(1H-tetrazol-1-yl)phenyl)ethyl (4-(((6-cyclopropylimidazo [1,2-a]pyridin-2-yl)methyl)amino)pyridin-2-yl)carbamate (5.9 mg, 16%) as a white solid. ESI-MS [M+H]⁺: 530.2. ¹H NMR (400 MHz, DMSO) δ 9.91 (s, 1H), 9.78 (s, 1H), 8.29 (s, 1H), 7.87 (d, J=2.1 Hz, 1H), 7.70-7.66 (m, 3H), 7.60 (s, 1H), 7.37 (d, J=9.3 Hz, 1H), 7.10 (t, J=5.5 Hz, 1H), 6.97-6.94 (m, 2H), 6.27 (dd, J=5.8, 2.0 Hz, 1H), 5.39 (q, J=6.5 Hz, 1H), 4.31 (d, J=5.7 Hz, 2H), 1.92-1.87 (m, 1H), 1.43 (d, J=6.6 Hz, 3H), 0.92-0.88 (m, 2H), 0.68-0.64 (m, 2H).

### Example 22

### Synthesis of 5-chloro-2-(1H-tetrazol-1-yl)benzyl (2-((6-cyclopropylimidazo[1,2-a]pyridin-2-yl)methyl)-2H-pyrazolo[4,3-c]pyridin-4-yl)carbamate (1-22)

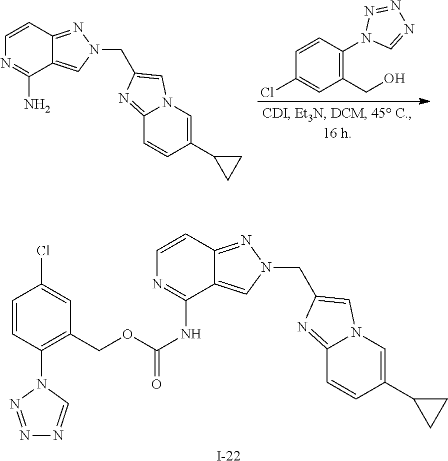

### Synthesis of 5-chloro-2-(1H-tetrazol-1-yl)benzyl (2-((6-cyclopropylimidazo[1,2-a]pyridin-2-yl)methyl)-2H-pyrazolo[4,3-c]pyridin-4-yl)carbamate

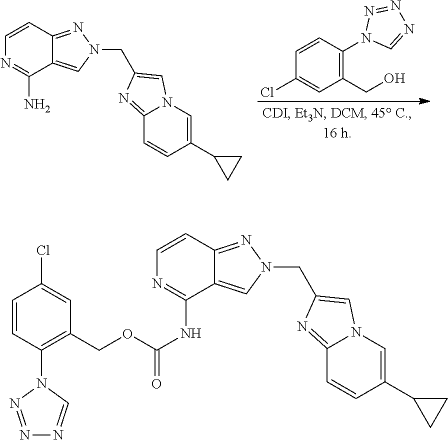 [SMILES: Nc1nccc2nn(Cc3cn4cc(C5CC5)ccc4n3)cc12 ; OCc1cc(Cl)ccc1-n1cnnn1 ; O=C(Nc1nccc2nn(Cc3cn4cc(C5CC5)ccc4n3)cc12)OCc1cc(Cl)ccc1-n1cnnn1]

A mixture of 2-((6-cyclopropylimidazo[1,2-a]pyridin-2-yl)methyl)-2H-pyrazolo[4,3-c]pyridin-4-amine (10 mg, 0.033 mmol), CDI (16 mg, 0.1 mmol), and Et₃N (10 mg, 0.1 mmol) in DCM (2 mL) was stirred at 45° C. for 16 h. (5-chloro-2-(1H-tetrazol-1-yl)phenyl)methanol (35 mg, 0.17 mmol) was added. The mixture was stirred at 45° C. for another 2 h. The reaction was quenched with water (30 mL) and extracted with DCM (10 mL×3). The combined organic layers were washed with water (30 mL), dried over anhydrous Na₂SO₄, and concentrated in vacuo to give the crude, which was purified by Prep-TLC (eluent: DCM/MeOH=10/1) to give 5-chloro-2-(1H-tetrazol-1-yl)benzyl (2-((6-cyclopropylimidazo[1,2-a]pyridin-2-yl)methyl)-2H-pyrazolo[4,3-c]pyridin-4-yl)carbamate (1.6 mg, 9%) as a white solid. ESI-MS [M+H]⁺: 541.1. ¹H NMR (400 MHz, DMSO) δ 10.57 (s, 1H), 9.91 (s, 1H), 8.73 (s, 1H), 8.34 (s, 1H), 7.91-7.73 (m, 5H), 7.39 (d, J=9.3 Hz, 1H), 7.30-6.99 (m, 2H), 5.71 (s, 2H), 5.08 (s, 2H), 1.95-1.89 (m, 1H), 0.94-0.89 (m, 2H), 0.69-0.65 (m, 2H).

### Example 23

### Synthesis of 5-chloro-2-(1H-tetrazol-1-yl)benzyl (1-((6-cyclopropylimidazo[1,2-a]pyridin-2-yl)methyl)-1H-imidazo[4,5-c]pyridin-6-yl)carbamate (I-23)

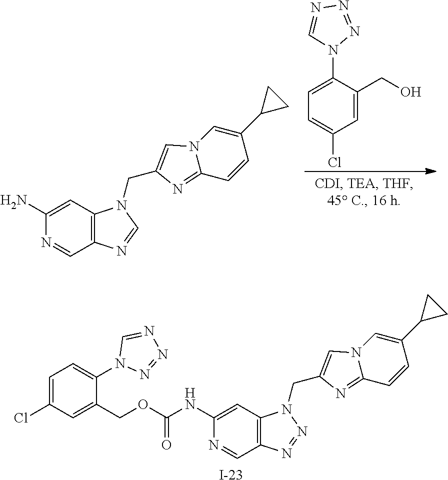

Synthesis of 5-chloro-2-(1H-tetrazol-1-yl)benzyl (1-((6-cyclopropylimidazo[1,2-a]pyridin-2-yl)methyl)-1H-imidazo[4,5-c]pyridin-6-yl)carbamate. A mixture of 1-((6-cyclopropylimidazo[1,2-a]pyridin-2-yl)methyl)-1H-imidazo[4,5-c]pyridin-6-amine (49 mg, 0.16 mmol), CDI (162 mg, 1 mmol), and TEA (101 mg, 1 mmol) in THF (5 mL) was stirred at 45° C. for 16 h. (5-chloro-2-(1H-tetrazol-1-yl)phenyl)methanol (210 mg, 1 mmol) was added and the mixture was stirred at 45° C. for another 2 h. The reaction was quenched with water (30 mL) and extracted with EtOAc (10 mL×3). The combined organic layers were washed with brine (30 mL), dried over anhydrous Na₂SO₄, and concentrated in vacuo to give the crude, which was purified by Prep-HPLC to give 5-chloro-2-(1H-tetrazol-1-yl)benzyl (1-((6-cyclopropylimidazo[1,2-a]pyridin-2-yl)methyl)-1H-imidazo[4,5-c]pyridin-6-yl)carbamate (10 mg, 12%) as a white solid. ESI-MS [M+H]+: 541.2. ¹H NMR (400 MHz, DMSO) δ=10.30 (s, 1H), 9.92 (s, 1H), 8.65 (s, 1H), 8.40 (s, 1H), 8.33 (s, 1H), 7.91 (s, 2H), 7.73 (s, 3H), 7.36 (d, J=9.4, 1H), 6.99-6.97 (m, 1H), 5.53 (s, 2H), 5.05 (s, 2H), 1.94-1.85 (m, 1H), 0.93-0.87 (m, 2H), 0.67-0.62 (m, 2H).

### Example 24

### Synthesis of 1-(3-chlorobenzyl)-3-(6-(((6-cyclopropylimidazo[1,2-a]pyridin-2-yl)methyl)amino)pyrimidin-4-yl)urea (I-24)

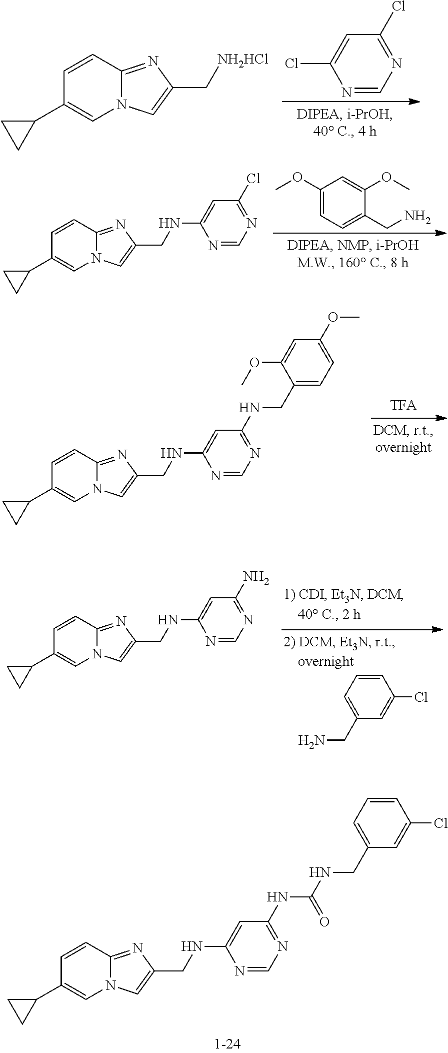

### Synthesis of 6-chloro-N-((6-cyclopropylimidazo[1,2-a]pyridin-2-yl)methyl)pyrimidin-4-amine

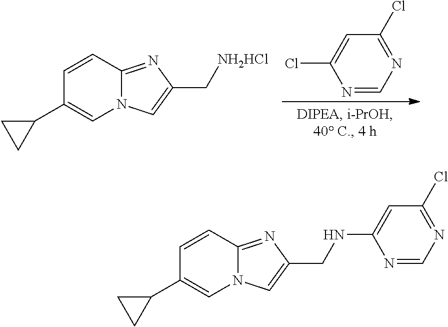

To a solution of (6-cyclopropylimidazo[1,2-a]pyridin-2-yl)methanamine hydrochloride (4.6 g, 21 mmol) and 4,6-dichloropyrimidine (4.6 g, 31 mmol) in iPrOH (48 mL) was added DIPEA (8.0 g, 62 mmol). The mixture was stirred at 40° C. for 4 h. The reaction mixture was concentrated in vacuo and the residue was purified by silica gel column (eluting with 0˜-5% MeOH/DCM) to afford 6-chloro-N-((6-cyclopropylimidazo[1,2-a]pyridin-2-yl)methyl)pyrimidin-4-amine (4.2 g, 67%) as a pale solid. ESI-MS [M+H]⁺: 300.2.

### Synthesis of N⁴-((6-cyclopropylimidazo[1,2-a]pyridin-2-yl)methyl)-N6-(2,4-dimethoxybenzyl) pyrimidine-4,6-diamine

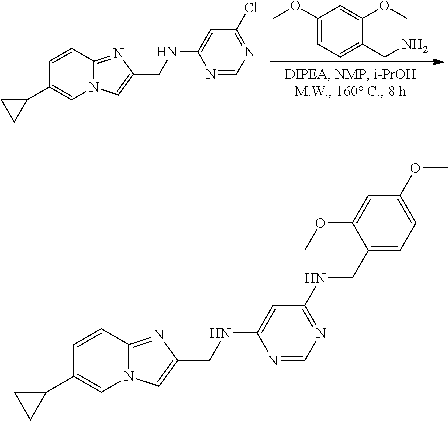

A solution of 6-chloro-N-((6-cyclopropylimidazo[1,2-a]pyridin-2-yl)methyl)pyrimidin-4-amine (1.4 g, 4.7 mmol), (2,4-dimethoxyphenyl)methanamine (1.2 g, 7.2 mmol), and DIPEA (1.8 g, 14 mmol) in i-PrOH (5.0 mL) and 1-methylpyrrolidin-2-one (5.0 mL) was stirred in a sealed tube. After degassing with N₂ for 1 min, the resulting mixture was irradiated in a microwave reactor at 160° C. for 8 h. The reaction mixture was then concentrated in vacuo to give the crude, which was purified by column chromatography on silica gel, eluting with 0˜10% MeOH/DCM to afford N⁴-((6-cyclopropylimidazo[1,2-a]pyridin-2-yl)methyl)-N6-(2,4-dimethoxybenzyl)pyrimidine-4,6-diamine (0.79 g, 39%) as a yellow soild. ESI-MS [M+H]⁺: 431.2.

### Synthesis of N⁴-((6-cyclopropylimidazo[1,2-a]pyridin-2-yl)methyl)pyrimidine-4,6-diamine

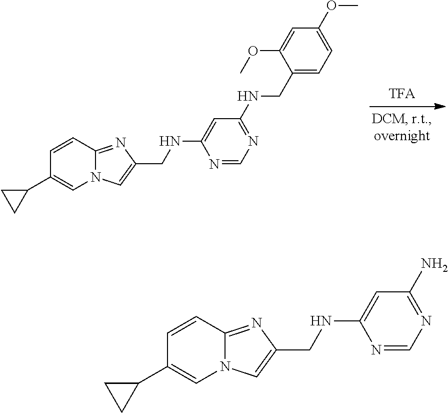 [SMILES: COc1ccc(CNc2cc(NCc3cn4cc(C5CC5)ccc4n3)ncn2)c(OC)c1 ; Nc1cc(NCc2cn3cc(C4CC4)ccc3n2)ncn1 ; "TFA"]

To a mixture of N⁴-((6-cyclopropylimidazo[1,2-a]pyridin-2-yl)methyl)-N6-(2,4-dimethoxybenz yl)pyrimidine-4,6-diamine (0.79 g, 1.8 mmol) in DCM (40 mL) was added TFA (8.2 mL, 0.11 mol). After stirring at room temperature overnight, the resulting mixture was concentrated in vacuo to give the crude. NH₃ (7.0 N in MeOH) was added to adjust the pH to 12. The resulting mixture was concentrated in vacuo and the residue was purified by silica gel column chromatography, eluting with 0˜-10% MeOH/DCM to give N⁴-((6-cyclopropylimidazo[1,2-a]pyridin-2-yl)methyl)pyrimidine-4,6-diamine (0.35 g, 70%) as a pale solid. ESI-MS [M+H]⁺: 281.1.

### Synthesis of 1-(3-chlorobenzyl)-3-(6-(((6-cyclopropylimidazo[1,2-a]pyridin-2-yl)methyl)amino)pyrimidin-4-yl)urea

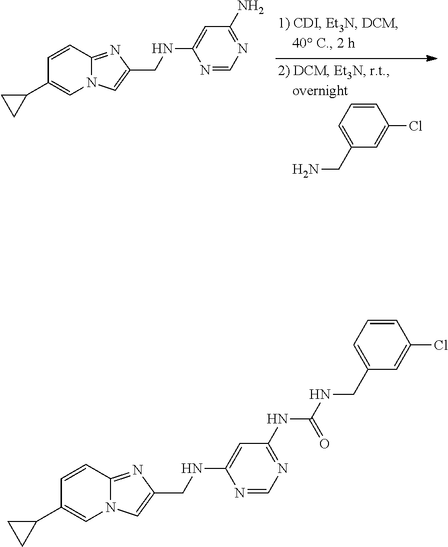 [SMILES: Nc1cc(NCc2cn3cc(C4CC4)ccc3n2)ncn1 ; NCc1cccc(Cl)c1 ; O=C(NCc1cccc(Cl)c1)Nc1cc(NCc2cn3cc(C4CC4)ccc3n2)ncn1]

To a solution of N⁴-((6-cyclopropylimidazo[1,2-a]pyridin-2-yl)methyl)pyrimidine-4,6-diamine (0.10 g, 0.36 mmol) and CDI (0.29 g, 1.8 mmol) in DCM (3.0 mL) was added Et₃N (0.36 g, 3.6 mmol) under nitrogen. After stirring at 40° C. for 2 h, a solution of (3-chlorophenyl)methanamine (0.25 g, 1.8 mmol) in DCM (3.0 mL) was added. The resulting mixture was stirred at room temperature overnight. The mixture was concentrated in vacuo, and the residue was purified by Prep-TLC (DCM/MeOH=10/1) to afford 1-(3-chlorobenzyl)-3-(6-(((6-cyclopropylimidazo[1,2-a]pyridin-2-yl)methyl)amino)pyrimidin-4-yl)urea (11 mg, 6.8%) as a pale solid. ESI-MS [M+H]⁺: 448.1, ¹H NMR (400 MHz, DMSO) δ 9.13 (s, 1H), 8.28-8.21 (m, 2H), 8.13 (s, 1H), 7.72 (s, 1H), 7.59 (s, 1H), 7.34-7.24 (m, 5H), 6.95 (d, J=10.9 Hz, 1H), 6.57 (s, 1H), 4.53 (d, J=9.4 Hz, 2H), 4.35 (d, J=6.0 Hz, 2H), 1.93-1.87 (m, 1H), 0.92-0.88 (m, 2H), 0.67-0.63 (m, 2H).

### Example 25

### Synthesis of 3-(3-chlorophenyl)-N-(6-(((6-cyclopropylimidazo[1,2-a]pyridin-2-yl)methyl)amino)pyrimidin-4-yl)azetidine-1-carboxamide (1-25)

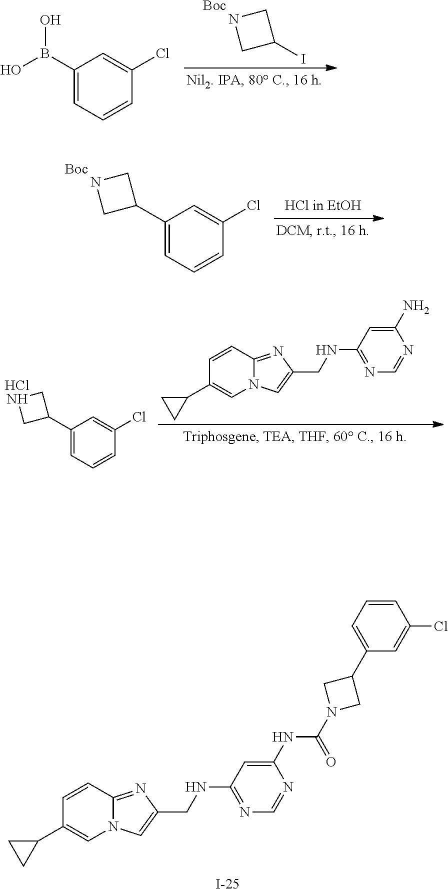

### Synthesis of tert-butyl 3-(3-chlorophenyl)azetidine-1-carboxylate

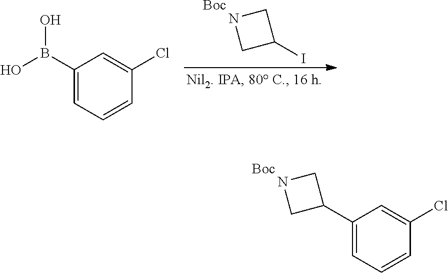 [SMILES: OB(O)c1cccc(Cl)c1 ; IC1CNC1 ; Clc1cccc(C2CNC2)c1 ; "Boc" ; "Boc"]

To a solution of 3-chlorophenyl)boronic acid (936 mg, 6.0 mmol) and tert-butyl 3-iodoazetidine-1-carboxylate (1.7 g, 6.0 mmol) in IPA (20 mL) was added NiI₂ (373 mg, 1.2 mmol). The reaction mixture was stirred at 80° C. for 16 h. The reaction was diluted with water (50 mL) and extracted with EtOAc (30 mL×3). The combined organic layers were washed with brine (30 mL), dried over anhydrous Na₂SO₄, and concentrated in vacuo to give tert-butyl 3-(3-chlorophenyl)azetidine-1-carboxylate (360 mg) as a light yellow solid which was used without further purification. ESI-MS [M+H]+: 268.1.

### Synthesis of 3-(3-chlorophenyl)azetidine

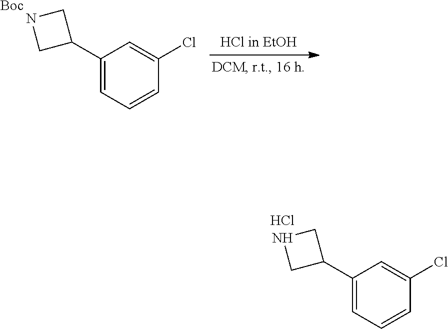 [SMILES: Clc1cccc(C2CNC2)c1 ; Cl ; Clc1cccc(C2CNC2)c1 ; "Boc"]

To a solution of tert-butyl 3-(3-chlorophenyl)azetidine-1-carboxylate (360 mg) in DCM (10 mL) was added HCl (2.0 M solution in EtOH, 2 mL). The resulting mixture was stirred at room temperature for 16 h. The reaction was concentrated in vacuo to give 3-(3-chlorophenyl)azetidine as the hydrochloric acid salt (250 mg) as a white solid, which was used without further purification. ESI-MS [M+H]⁺: 168.2

### Synthesis of 3-(3-chlorophenyl)-N-(6-(((6-cyclopropylimidazo[1,2-a]pyridin-2-yl)methyl)amino)pyrimidin-4-yl)azetidine-1-carboxamide

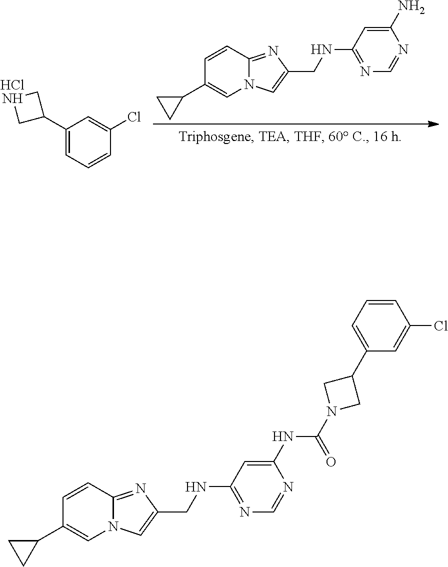

To a solution of 3-(3-chlorophenyl)azetidine hydrochloride (50 mg, crude), N⁴-((6-cyclopropylimidazo[1,2-a]pyridin-2-yl)methyl)pyrimidine-4,6-diamine (50 mg, 0.18 mmol), and TEA (55 mg, 0.54 mmol) in THF (10 mL) was added triphosgene (35 mg, 0.12 mmol). The reaction mixture was stirred at 60° C. for 16 h. After cooling to 25° C., water (50 mL) was added and the mixture was extracted with EtOAc (30 mL×3). The combined organic layers were washed with brine (60 mL), dried over anhydrous Na₂SO₄, and concentrated in vacuo to give the crude, which was purified by column chromatography (eluent: DCM/MeOH=20/1) to give 3-(3-chlorophenyl)-N-(6-(((6-cyclopropylimidazo[1,2-a]pyridin-2-yl)methyl)amino)pyrimidin-4-yl)azetidine-1-carboxamide (15 mg, yield: 18%) as a white solid. ESI-MS [M+H]⁺: 474.2. ¹H NMR (400 MHz, DMSO) δ 8.42 (s, 1H), 8.30 (s, 1H), 7.80 (s, 1H), 7.53 (s, 1H), 7.42-7.18 (t, J=16.2 Hz, 5H), 6.36 (s, 1H), 4.94 (s, 2H), 4.20 (s, 2H), 3.77 (s, 3H), 2.05-1.94 (m, 1H), 0.99-0.93 (m, 2H), 0.75-0.65 (m, 2H).

### Example 26

### Synthesis of (4-chloropyridin-2-yl)methyl (6-(((6-cyclopropylimidazo[1,2-a]pyridin-2-yl)methyl)amino)pyrimidin-4-yl)carbamate (I-26)

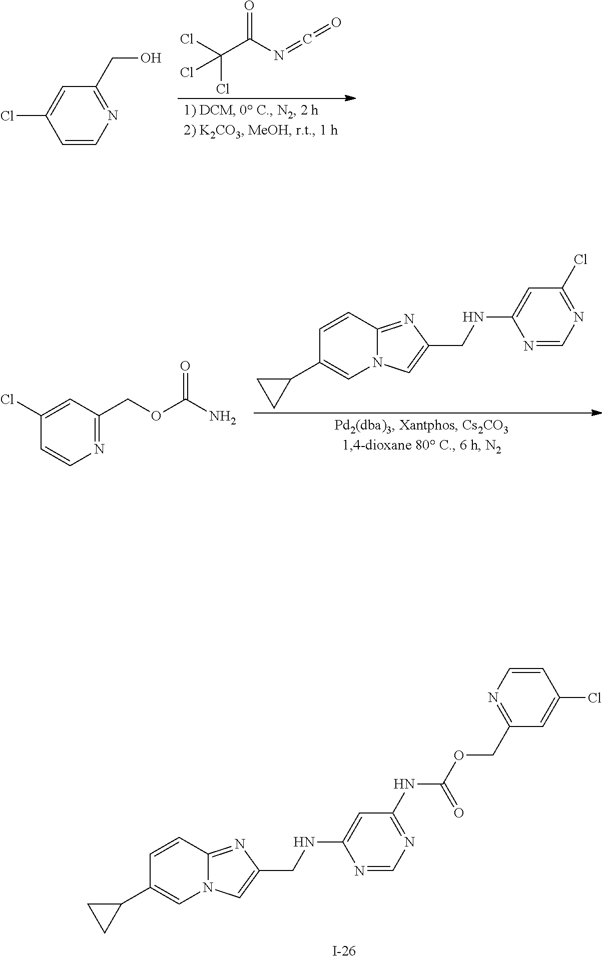

### Synthesis of (4-chloropyridin-2-yl)methyl carbamate

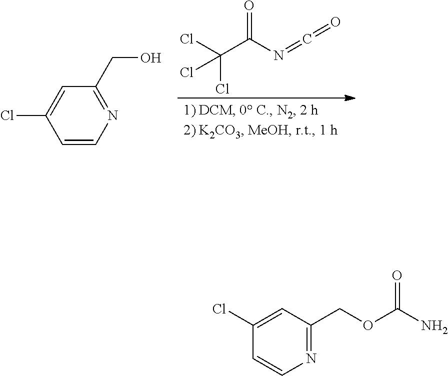 [SMILES: OCc1cc(Cl)ccn1 ; O=C=NC(=O)C(Cl)(Cl)Cl ; NC(=O)OCc1cc(Cl)ccn1]

To a mixture of (4-chloropyridin-2-yl)methanol (0.5 g, 3.5 mmol) in DCM (10 mL) was added 2,2,2-trichloroacetyl isocyanate (0.79 g, 4.2 mmol) at 0° C. The mixture was stirred at 0° C. for 2 h. It was then was concentrated in vacuo, and the residue was re-dissolved in MeOH (10 mL). K₂CO₃ (1.4 g, 10.5 mmol) was added. The mixture was stirred at room temperature for 1 h. The mixture was diluted with water (50 mL) and extracted with EtOAc (30 mL×3). The combined organic layers were washed with brine (30 mL), dried over Na₂SO₄, and concentrated in vacuo to give the crude product. The crude product was purified by column chromatography on silica gel (eluent: EtOAc/PE=0-50%) to give (4-methylpyrimidin-2-yl)methyl carbamate (0.46 g, yield 71%) as a white solid. ESI-MS [M+H]+: 187.1.

### Synthesis of (4-chloropyridin-2-yl)methyl (6-(((6-cyclopropylimidazo[1,2-a]pyridin-2-yl)methyl)amino)pyrimidin-4-yl)carbamate

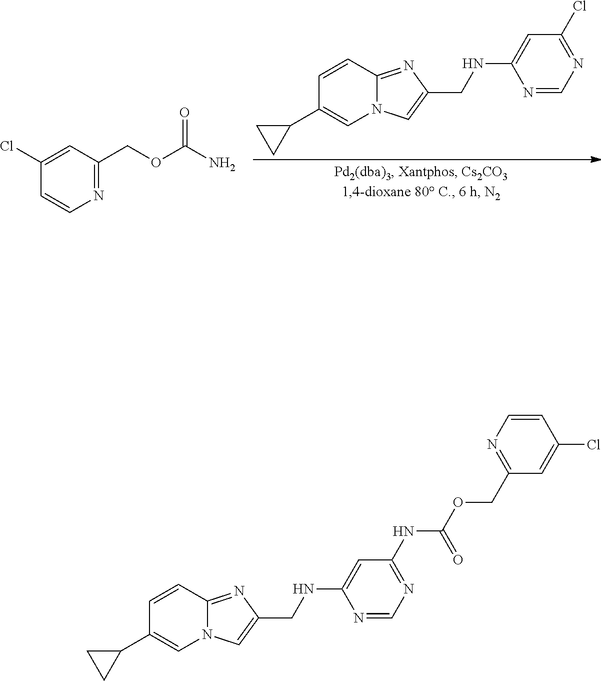 [SMILES: NC(=O)OCc1cc(Cl)ccn1 ; Clc1cc(NCc2cn3cc(C4CC4)ccc3n2)ncn1 ; O=C(Nc1cc(NCc2cn3cc(C4CC4)ccc3n2)ncn1)OCc1cc(Cl)ccn1]

A mixture of (4-chloropyridin-2-yl)methyl carbamate (75 mg, 0.40 mmol), 6-chloro-N-((6-cyclopropylimidazo[1,2-a]pyridin-2-yl)methyl)pyrimidin-4-amine (0.10 g, 0.33 mmol), Pd₂(dba)₃ (45 mg, 0.050 mmol), Xantphos (29 mg, 0.050 mmol), and Cs₂CO₃ (0.14 g, 1.0 mmol) in 1,4-dioxane (5 mL) was stirred at 80° C. for 6 h under N₂. After cooling to room temperature, the mixture was filtered through Celite® and the filter cake was washed with DCM/MeOH (10/1, 50 mL). The filtrate was concentrated in vacuo to give the crude, which was purified by Prep-TLC (eluent: DCM/MeOH=8/1) to afford (4-chloropyridin-2-yl)methyl (6-(((6-cyclopropylimidazo[1,2-a]pyridin-2-yl)methyl)amino)pyrimidin-4-yl)carbamate (4.3 mg, yield 2.4%) as pale-yellow solid. ESI-MS [M+H]+: 450.2, ¹H NMR (400 MHz, DMSO) δ 10.30 (s, 1H), 8.54 (d, J=5.3 Hz, 1H), 8.29 (s, 1H), 8.18 (s, 1H), 7.85 (s, 1H), 7.65 (s, 1H), 7.61 (s, 1H), 7.50 (dd, J=5.3, 1.9 Hz, 1H), 7.36 (d, J=9.3 Hz, 1H), 7.01 (s, 1H), 6.95 (dd, J=9.3, 1.6 Hz, 1H), 5.21 (s, 2H), 4.56 (s, 2H), 1.94-1.87 (m, 1H), 0.93-0.87 (m, 2H), 0.67-0.64 (m, 2H).

### Example 27

### Synthesis of 1-((4-chloropyridin-2-yl)methyl)-3-(6-(((6-cyclopropylimidazo[1,2-a]pyridin-2-yl)methyl)amino)pyrimidin-4-yl)urea (I-27)

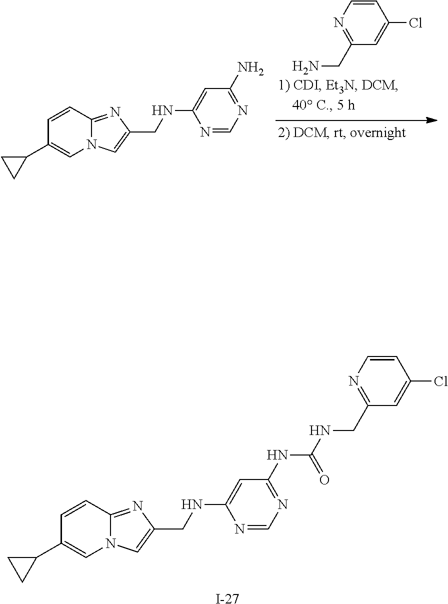

Synthesis of 1-((4-chloropyridin-2-yl)methyl)-3-(6-(((6-cyclopropylimidazo[1,2-a]pyridin-2-yl)methyl)amino)pyrimidin-4-yl)urea. To a solution of N⁴-((6-cyclopropylimidazo[1,2-a]pyridin-2-yl)methyl)pyrimidine-4,6-diamine (50 mg, 0.18 mmol) in DCM (3.0 mL) was added di(1H-imidazol-1-yl)methanone (87 mg, 0.54 mmol) and Et₃N (0.11 g, 1.1 mmol). After stirring at 40° C. for 5 h, a solution of (4-chloropyridin-2-yl)methanamine (76 mg, 0.53 mmol) in DCM (1.0 mL) was added. The resulting mixture was stirred at room temperature overnight. The mixture was concentrated in vacuo and the residue was purified by Prep-HPLC to give 1-((4-chloropyridin-2-yl)methyl)-3-(6-(((6-cyclopropylimidazo[1,2-a]pyridin-2-yl)methyl)amino)pyrimidin-4-yl)urea (4.2 mg, 5.2%) as a white solid. ESI-MS [M+H]+: 449.2. ¹H NMR (400 MHz, DMSO) δ=9.22 (s, 1H), 8.50 (d, J=5.1 Hz, 1H), 8.40 (s, 1H), 8.29 (s, 1H), 8.14 (s, 1H), 7.72 (s, 1H), 7.60 (s, 1H), 7.48-7.39 (m, 2H), 7.37-7.35 (m, 1H), 6.95 (d, J=10.0 Hz, 1H), 6.58 (s, 1H), 4.53 (s, 2H), 4.45 (d, J=5.6 Hz, 2H), 1.94-1.87 (m, 1H), 0.93-0.84 (m, 2H), 0.69-0.60 (m, 2H).

### Example 28

### Synthesis of (4-chloropyridin-2-yl)methyl (6-((2R,4S)-2-(6-cyclopropylimidazo[1,2-a]pyridin-2-yl)-4-hydroxypyrrolidin-1-yl)pyrimidin-4-yl)carbamate (I-28)

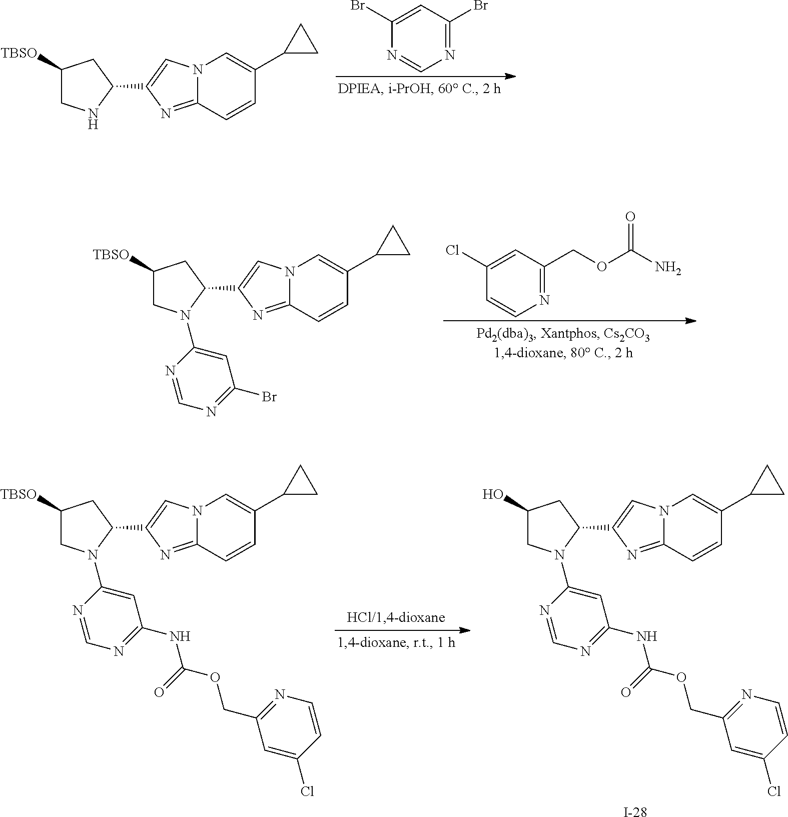

### Synthesis of 2-((2R,4S)-1-(6-bromopyrimidin-4-yl)-4-((tert-butyldimethylsilyl)oxy)pyrrolidin-2-yl)-6-cyclopropylimidazo[1,2-a]pyridine

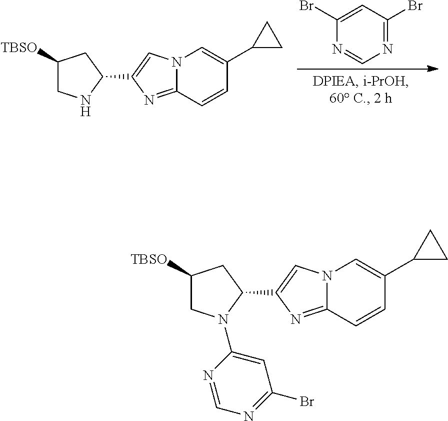 [SMILES: CC(C)(C)[Si](C)(C)O[C@@H]1CN[C@@H](c2cn3cc(C4CC4)ccc3n2)C1 ; Brc1cc(Br)ncn1 ; CC(C)(C)[Si](C)(C)O[C@H]1C[C@H](c2cn3cc(C4CC4)ccc3n2)N(c2cc(Br)ncn2)C1]

To a solution of 2-((2R,4S)-4-((tert-butyldimethylsilyl)oxy)pyrrolidin-2-yl)-6-cyclopropylimidazo[1,2-a]pyridine (714 mg, 2 mmol) and 4,6-dibromopyrimidine (569 mg, 2.4 mmol) in iPrOH (10 mL) was added DIPEA (775 mg, 6 mmol). The mixture was stirred at 60° C. for 2 h. The reaction mixture was concentrated in vacuo and the residue was purified by column purification on silica gel (eluting with 0˜-5% MeOH/DCM) to afford 2-((2R,4S)-1-(6-bromopyrimidin-4-yl)-4-((tert-butyldimethylsilyl)oxy)pyrrolidin-2-yl)-6-cyclopropylimidazo[1,2-a]pyridine (617 mg, 60%) as a yellow solid. ESI-MS [M+H]⁺: 515.2.

### Synthesis of (4-chloropyridin-2-yl)methyl (6-((2R,4S)-4-((tert-butyldimethylsilyl)oxy)-2-(6-cyclopropylimidazo[1,2-a]pyridin-2-ylpyrrolidin-1-yl)pyrimidin-4-yl)carbamate

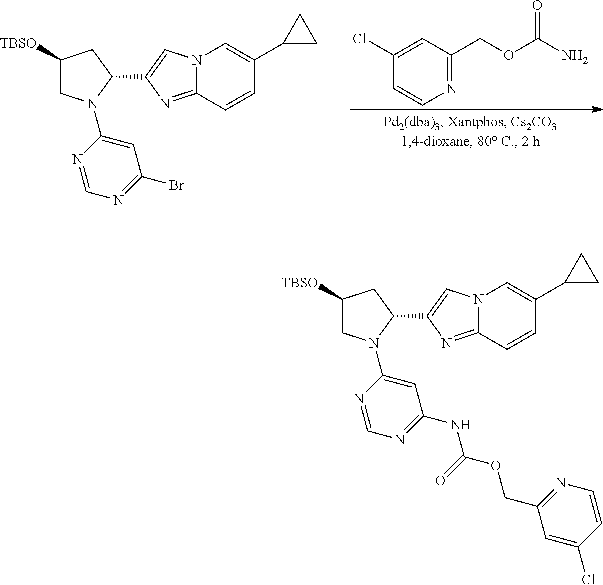 [SMILES: CC(C)(C)[Si](C)(C)O[C@H]1C[C@H](c2cn3cc(C4CC4)ccc3n2)N(c2cc(Br)ncn2)C1 ; NC(=O)OCc1cc(Cl)ccn1 ; CC(C)(C)[Si](C)(C)O[C@H]1C[C@H](c2cn3cc(C4CC4)ccc3n2)N(c2cc(NC(=O)OCc3cc(Cl)ccn3)ncn2)C1]

To a solution of 2-((2R,4S)-1-(6-bromopyrimidin-4-yl)-4-((tert-butyldimethylsilyl)oxy)pyrrolidin-2-yl)-6-cyclopropylimidazo[1,2-a]pyridine (0.15 g, 0.29 mmol), (4-chloropyridin-2-yl)methyl carbamate (65 mg, 0.35 mmol), and Cs₂CO₃ (0.28 g, 0.86 mmol) in 1,4-dioxane (2.0 mL) was added Pd₂(dba)₃ (26 mg, 0.028 mmol) and Xantphos (16 mg, 0.028 mmol). The reaction mixture was stirred at 80° C. for 2 h under nitrogen. The reaction mixture was filtered through Celite® and the filter cake was washed with DCM/MeOH (10/1, 20 mL). The filtrate was concentrated in vacuo to give the crude, which was purified by column chromatography on silica gel, eluting with 20-100% EA/PE to afford (4-chloropyridin-2-yl)methyl (6-((2R, 4S)-4-((tert-butyldimethylsilyl)oxy)-2-(6-cyclopropylimidazo[1,2-a]pyridin-2-yl)pyrrolidin-1-yl)pyrimidin-4-yl)carbamate (80 mg, 44%) as a yellow solid. ESI-MS [M+H]⁺: 620.2.

### Synthesis of (4-chloropyridin-2-yl)methyl (6-((2R,4S)-2-(6-cyclopropylimidazo[1,2-a]pyridin-2-yl)-4-hydroxypyrrolidin-1-yl)pyrimidin-4-yl)carbamate

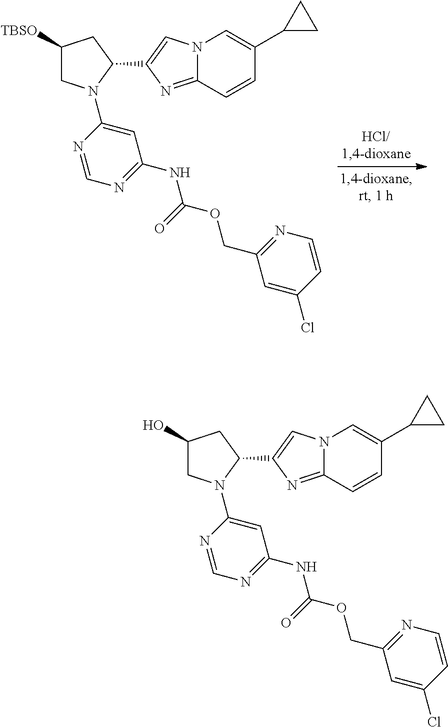 [SMILES: CC(C)(C)[Si](C)(C)O[C@H]1C[C@H](c2cn3cc(C4CC4)ccc3n2)N(c2cc(NC(=O)OCc3cc(Cl)ccn3)ncn2)C1 ; O=C(Nc1cc(N2C[C@@H](O)C[C@@H]2c2cn3cc(C4CC4)ccc3n2)ncn1)OCc1cc(Cl)ccn1]

To a mixture of (4-chloropyridin-2-yl)methyl (6-((2R, 4S)-4-((tert-butyldimethylsilyl)oxy)-2-(6-cyclopropylimidazo[1,2-a]pyridin-2-yl)pyrrolidin-1-yl)pyrimidin-4-yl)carbamate (0.25 g, 0.40 mmol) in 1,4-dioxane (1.0 mL) was added HCl (1.0 mL, 4 N in 1,4-dioxane) at room temperature. After stirring at room temperature for 1 h, the resulting mixture was concentrated in vacuo and purified by Prep-HPLC to afford (4-chloropyridin-2-yl)methyl (6-((2R,4S)-2-(6-cyclopropylimidazo[1,2-a]pyridin-2-yl)-4-hydroxypyrrolidin-1-yl)pyrimidin-4-yl)carbamate (30 mg, yield:15%) as a pale solid. ESI-MS [M+H]⁺: 506.1, ¹H NMR (400 MHz, CD₃OD) δ 8.45 (d, J=5.3 Hz, 1H), 8.24-8.10 (m, 2H), 7.63 (s, 1H), 7.51 (s, 1H), 7.42-7.35 (m, 2H), 7.08 (d, J=8.4 Hz, 1H), 6.99 (s, 1H), 5.21 (s, 3H), 4.61-4.57 (m, 1H), 3.99-3.95 (m, 2H), 2.46-2.36 (m, 2H), 1.95-1.88 (m, 1H), 0.98-0.94 (m, 2H), 0.71-0.67 (m, 2H).

### Example 29

### Synthesis of 1-((4-chloropyrimidin-2-yl)methyl)-3-(6-(((6-cyclopropylimidazo[1,2-a]pyridin-2-yl)methyl)amino)-pyrimidin-4-yl)urea (I-29)

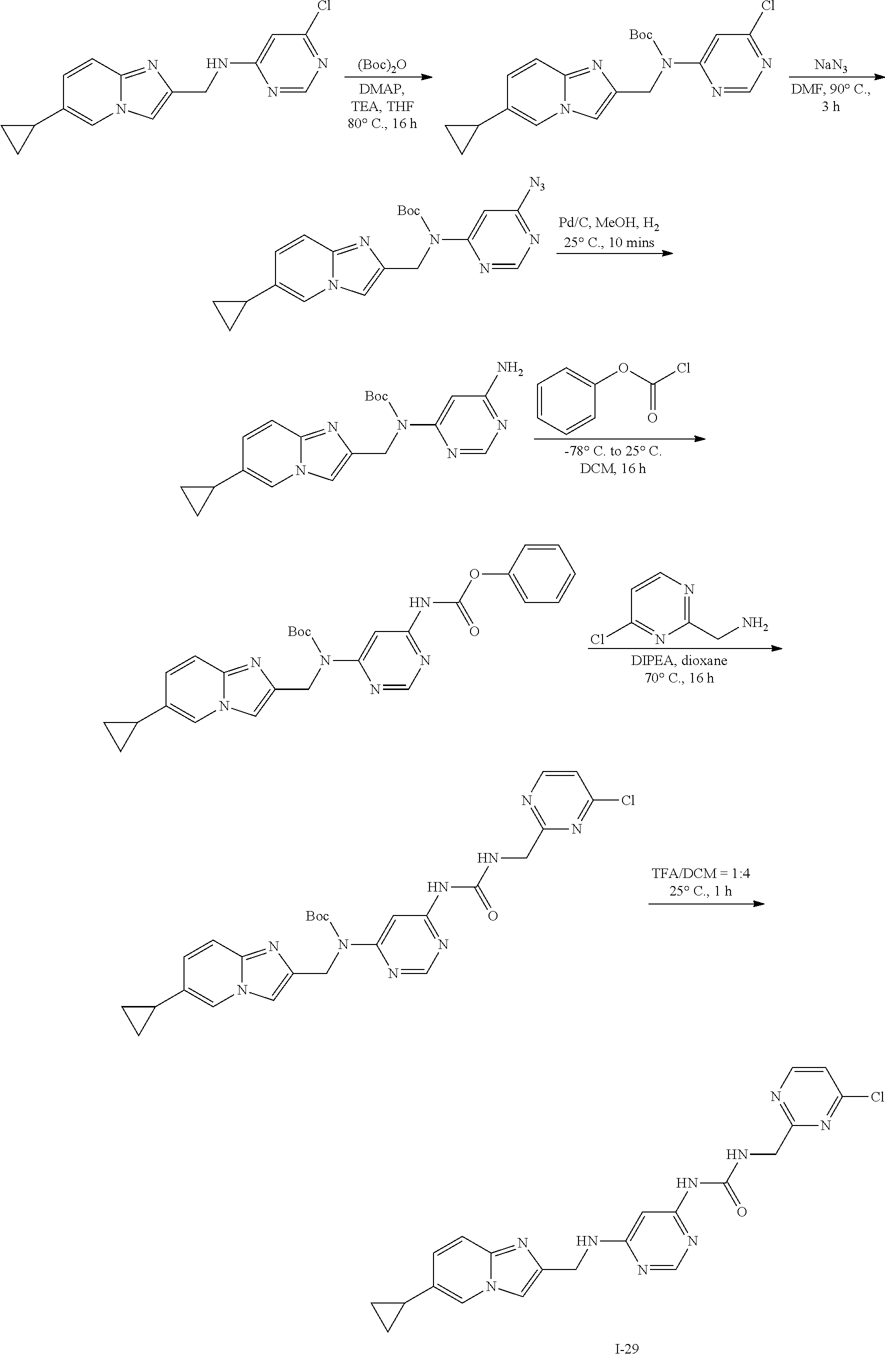

### Synthesis of tert-butyl (6-chloropyrimidin-4-yl)((6-cyclopropylimidazo[1,2-a]pyridin-2-yl)methyl)carbamate

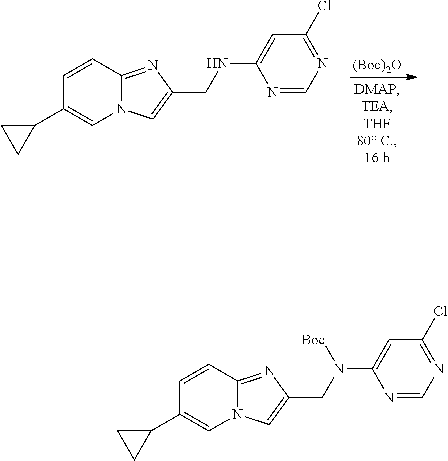 [SMILES: "(Boc)2O" ; Clc1cc(NCc2cn3cc(C4CC4)ccc3n2)ncn1 ; CC(C)(C)OC(=O)N(Cc1cn2cc(C3CC3)ccc2n1)c1cc(Cl)ncn1]

A mixture of 6-chloro-N-((6-cyclopropylimidazo[1,2-a]pyridin-2-yl)methyl)pyrimidin-4-amine (1 g, 3.36 mmol), (Boc)₂O (870 mg, 4 mmol), DMAP (81 mg, 0.66 mmol) and TEA (1.01 g, 10 mmol) in THF (30 mL) was stirred at 80° C. for 16 h. The mixture was concentrated in vacuo to give the crude product, which was purified by silica gel column chromatography (DCM:MeOH=10:1) to give the product (1 g, 74.4% yield) as a white solid. ESI-MS [M+H]⁺: 400.3.

### Synthesis of tert-butyl (6-azidopyrimidin-4-yl)((6-cyclopropylimidazo[1,2-a]pyridin-2-yl)methyl)carbamate

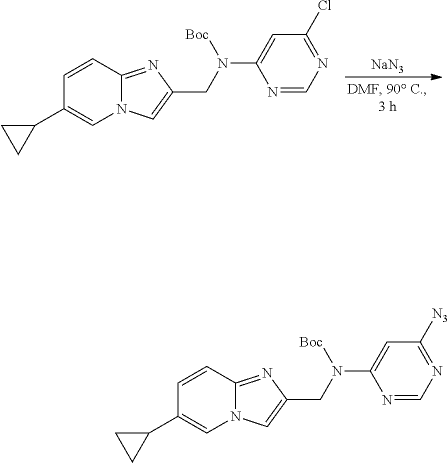 [SMILES: CC(C)(C)OC(=O)N(Cc1cn2cc(C3CC3)ccc2n1)c1cc(Cl)ncn1 ; CC(C)(C)OC(=O)N(Cc1cn2cc(C3CC3)ccc2n1)c1cc(N=[N+]=[N-])ncn1 ; "NaN3"]

A mixture of tert-butyl (6-chloropyrimidin-4-yl)((6-cyclopropylimidazo[1,2-a]pyridin-2-yl)methyl)carbamate (800 mg, 2.0 mmol) and NaN₃ (390 mg, 6.0 mmol) in DMF (30 mL) was stirred at 90° C. for 3 h. The mixture was diluted with H₂O (60 mL), then extracted with EtOAc (50 mL×3). The combined organic layers were washed with brine (50 mL), dried over Na₂SO₄, then concentrated to give the crude product (700 mg, crude), which was used in the next step without further purification. ESI-MS [M+H]+: 407.2.

### Synthesis of tert-butyl (6-aminopyrimidin-4-yl)((6-cyclopropylimidazo[1,2-a]pyridin-2-yl)methyl)carbamate

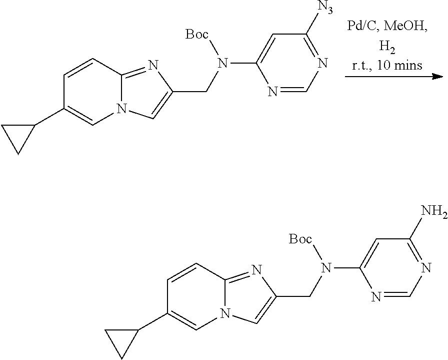 [SMILES: CC(C)(C)OC(=O)N(Cc1cn2cc(C3CC3)ccc2n1)c1cc(N=[N+]=[N-])ncn1 ; "Pd/C, MeOH,H2r.t., 10 mins" ; CC(C)(C)OC(=O)N(Cc1cn2cc(C3CC3)ccc2n1)c1cc(N)ncn1]

A mixture of tert-butyl (6-azidopyrimidin-4-yl)((6-cyclopropylimidazo[1,2-a]pyridin-2-yl)methyl)carbamate (700 mg, crude) and Pd/C (100 mg, 10%) in MeOH (10 mL) was stirred at room temperature for 10 min under H₂. The mixture was filtered and concentrated in vacuo to give the crude product (600 mg, crude) as a white solid, which was used in the next step without further purification. ESI-MS [M+H]+: 381.2.

### Synthesis of tert-butyl ((6-cyclopropylimidazo[1,2-a]pyridin-2-yl)methyl)(6-((phenoxycarbonyl)amino)pyrimidin-4-yl)carbamate

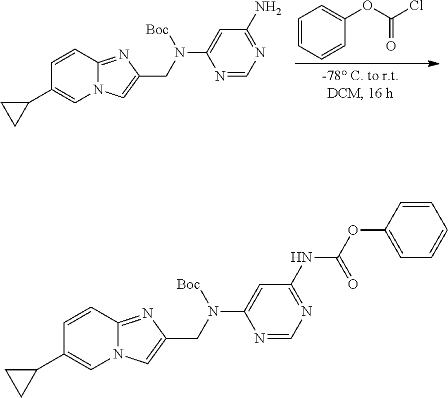

To a mixture of tert-butyl (6-aminopyrimidin-4-yl)((6-cyclopropylimidazo[1,2-a]pyridin-2-yl)methyl)carbamate (100 mg, crude) and DIPEA (170 mg, 1.31 mmol) in DCM (10 mL) was added phenyl carbonochloridate (82 mg, 0.525 mol) at −78° C. The mixture was warmed to room temperature and stirred for 16 h. The mixture was concentrated in vacuo to give the crude residue, which was purified by Prep-TLC (PE:EtOAc=1:1) to give the product (100 mg, 60% for 3 steps) as a white solid.

### Synthesis of tert-butyl (6-(3-((4-chloropyrimidin-2-yl)methyl)ureido)pyrimidin-4-yl)((6-cyclopropylimidazo[1,2-a]pyridin-2-yl)methyl)carbamate

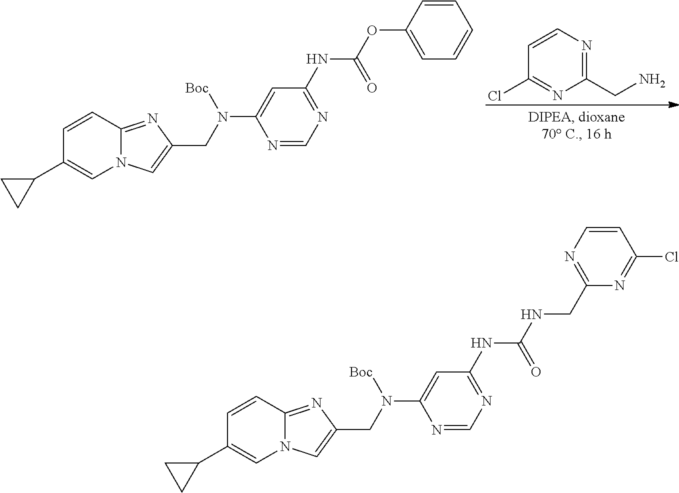 [SMILES: CC(C)(C)OC(=O)N(Cc1cn2cc(C3CC3)ccc2n1)c1cc(NC(=O)Oc2ccccc2)ncn1 ; NCc1nccc(Cl)n1 ; CC(C)(C)OC(=O)N(Cc1cn2cc(C3CC3)ccc2n1)c1cc(NC(=O)NCc2nccc(Cl)n2)ncn1]

To a mixture of tert-butyl ((6-cyclopropylimidazo[1,2-a]pyridin-2-yl)methyl)(6-((phenoxycarbonyl)amino)pyrimidin-4-yl)carbamate (50 mg, 0.1 mmol) and DIPEA (28.7 mg, 0.77 mmol) in 1,4-dioxane (5 mL) was added (4-chloropyrimidin-2-yl)methanamine (28.7 mg, 0.2 mmol). The mixture was stirred at 70° C. for 16 h. The mixture was concentrated in vacuo to give the crude product, which was purified by Prep-TLC (DCM:MeOH=10:1) to give the product (30 mg, 54.5%) as a white solid. ESI-MS [M+H]+: 550.2.

### Synthesis of 1-((4-chloropyrimidin-2-yl)methyl)-3-(6-(((6-cyclopropylimidazo[1,2-a]pyridin-2-yl)methyl)amino)pyrimidin-4-yl)urea

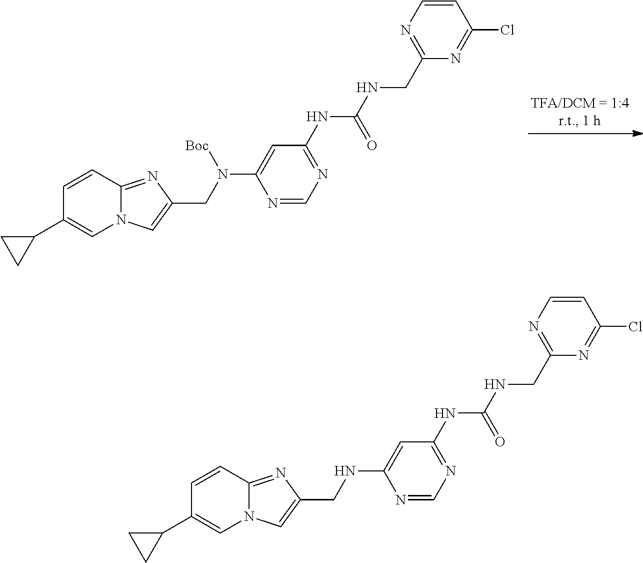 [SMILES: CC(C)(C)OC(=O)N(Cc1cn2cc(C3CC3)ccc2n1)c1cc(NC(=O)NCc2nccc(Cl)n2)ncn1 ; O=C(NCc1nccc(Cl)n1)Nc1cc(NCc2cn3cc(C4CC4)ccc3n2)ncn1]

To a solution of tert-butyl (6-(3-((4-chloropyrimidin-2-yl)methyl)ureido)pyrimidin-4-yl)((6-cyclopropylimidazo[1,2-a]pyridin-2-yl)methyl)carbamate (25 mg, 0.045 mmol) in DCM (4 mL) was added TFA (1 mL). The resulting mixture was stirred at room temperature for 1 h, then concentrated in vacuo. The residue was purified by Prep-TLC (DCM:MeOH=10:1) to give the product (12.3 mg, 60% yield) as a white solid. ESI-MS [M+H]⁺: 450.1. ¹H NMR (400 MHz, DMSO) δ 9.45-9.39 (m, 1H), 8.77 (d, J=5.4 Hz, 1H), 8.64 (s, 1H), 8.21 (s, 2H), 7.99 (s, 2H), 7.79-7.73 (m, 1H), 7.64-7.62 (m, 2H), 6.73 (s, 1H), 4.71 (s, 2H), 4.56 (d, J=5.7 Hz, 2H), 2.13-2.02 (m, 1H), 1.09-0.97 (m, 2H), 0.81-0.72 (m, 2H).

### Example 30

### Synthesis of 1-(6-(((6-cyclopropylimidazo[1,2-a]pyridin-2-yl)methyl)amino)pyrimidin-4-yl)-3-((4-methoxypyrimidin-2-yl)methyl)urea (I-30)

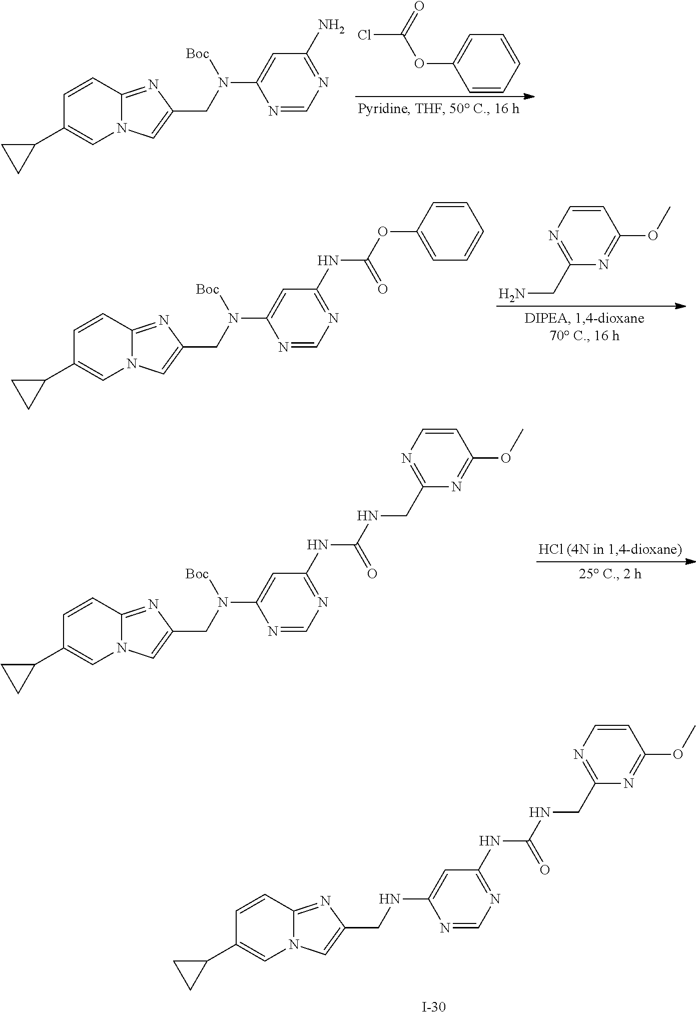

### Synthesis of tert-butyl ((6-cyclopropylimidazo[1,2-a]pyridin-2-yl)methyl)(6-((phenoxycarbonyl)amino)pyrimidin-4-yl)carbamate

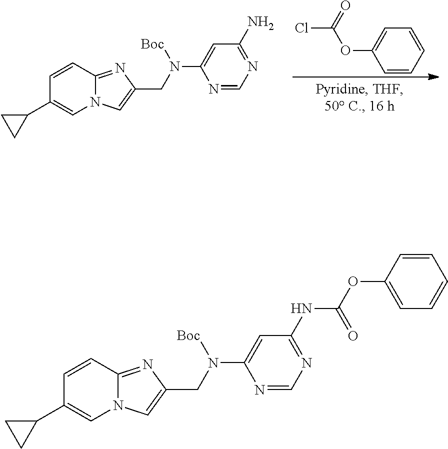 [SMILES: CC(C)(C)OC(=O)N(Cc1cn2cc(C3CC3)ccc2n1)c1cc(N)ncn1 ; O=C(Cl)Oc1ccccc1 ; CC(C)(C)OC(=O)N(Cc1cn2cc(C3CC3)ccc2n1)c1cc(NC(=O)Oc2ccccc2)ncn1]

A mixture of tert-butyl (6-aminopyrimidin-4-yl)((6-cyclopropylimidazo[1,2-a]pyridin-2-yl)methyl)carbamate (20 mg, 0.05 mmol), phenyl carbonochloridate (15 mg, 0.10 mmol), and pyridine (20 mg, 0.25 mmol) in THF (10 mL) was stirred at 50° C. for 16 h. The mixture was concentrated in vacuo to give the crude tert-butyl ((6-cyclopropylimidazo[1,2-a]pyridin-2-yl)methyl)(6-((phenoxycarbonyl)amino) pyrimidin-4-yl)carbamate (60 mg, crude) as a colorless oil, which was used for without further purification. ESI-MS [M+H]⁺: 501.2

### Synthesis of tert-butyl ((6-cyclopropylimidazo[1,2-a]pyridin-2-yl)methyl)(6-(3-((4-methoxypyrimidin-2-yl)methyl)ureido)pyrimidin-4-yl)carbamate

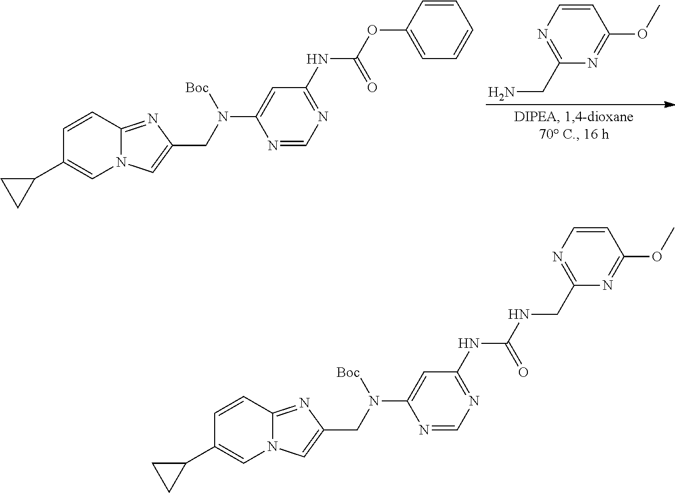

To a solution of tert-butyl ((6-cyclopropylimidazo[1,2-a]pyridin-2-yl)methyl)(6-((phenoxycarbonyl) amino)pyrimidin-4-yl)carbamate (60 mg, crude from previous step) in 1,4-dioxane (4.0 mL) was added a solution of (4-methoxypyrimidin-2-yl)methanamine (14 mg, 0.10 mmol) and DIPEA (0.13 g, 1.0 mmol) in 1,4-dioxane (2.0 mL). After stirring at 70° C. for 16 h, the resulting mixture was concentrated in vacuo to give the crude product, which was purified by Prep-TLC (DCM/MeOH=10/1) to give tert-butyl ((6-cyclopropylimidazo[1,2-a]pyridin-2-yl)methyl)(6-(3-((4-methoxypyrimidin-2-yl)methyl)ureido)pyrimidin-4-yl)carbamate (10 mg, 37%, 2 steps) as a white solid. ESI-MS [M+H]⁺: 546.2.

### Synthesis of 1-(6-(((6-cyclopropylimidazo[1,2-a]pyridin-2-yl)methyl)amino)pyrimidin-4-yl)-3-((4-methoxypyrimidin-2-yl)methyl)urea

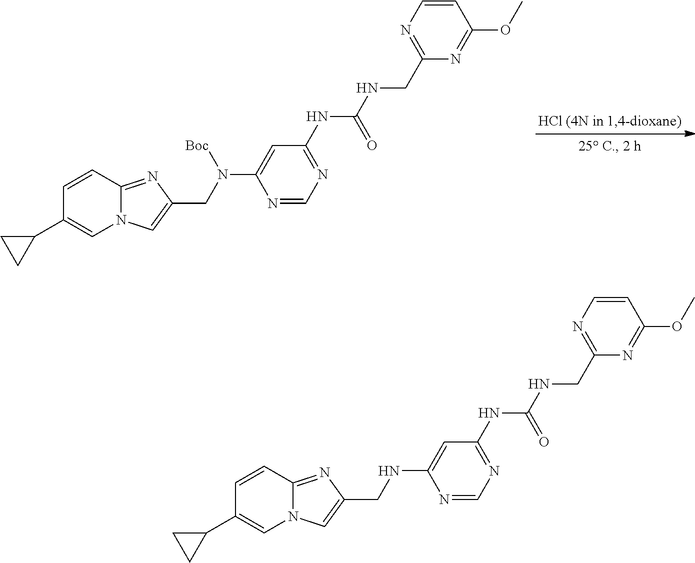 [SMILES: COc1ccnc(CNC(=O)Nc2cc(N(Cc3cn4cc(C5CC5)ccc4n3)C(=O)OC(C)(C)C)ncn2)n1 ; COc1ccnc(CNC(=O)Nc2cc(NCc3cn4cc(C5CC5)ccc4n3)ncn2)n1]

To a mixture of tert-butyl ((6-cyclopropylimidazo[1,2-a]pyridin-2-yl)methyl)(6-(3-((4-methoxypyrimidin-2-yl)methyl)ureido)pyrimidin-4-yl)carbamate (10 mg, 0.018 mmol) in 1,4-dioxane (4.0 mL) was added HCl (1.0 mL, 4 N in 1,4-dioxane) at 0° C. After stirring at 25° C. for 2 h, the resulting mixture was concentrated in vacuo to give the crude, which was purified by Prep-TLC (DCM/MeOH=10/1) to give 1-(6-(((6-cyclopropylimidazo[1,2-a]pyridin-2-yl)methyl)amino)pyrimidin-4-yl)-3-((4-methoxypyrimidin-2-yl) methyl)urea (1.3 mg, yield:16%) as a white solid. ESI-MS [M+H]+: 446.2. ¹H NMR (400 MHz, DMSO) δ 9.30 (s, 1H), 8.46 (d, J=5.8 Hz, 1H), 8.28 (s, 1H), 8.13 (s, 1H), 7.75-7.66 (m, 1H), 7.58 (s, 1H), 7.35 (d, J=9.3 Hz, 1H), 6.94 (dd, J=9.3, 1.6 Hz, 1H), 6.81 (d, J=5.8 Hz, 1H), 6.56-6.48 (m, 1H), 4.56-4.48 (m, 2H), 4.45 (d, J=5.4 Hz, 2H), 3.92 (s, 3H), 1.94-1.85 (m, 1H), 0.93-0.86 (m, 2H), 0.68-0.62 (m, 2H).

### Example 31

### Synthesis of N-(6-((2R,4S)-2-(6-cyclopropylimidazo[1,2-a]pyridin-2-yl)-4-hydroxypyrrolidin-1-yl)pyrimidin-4-yl)-3-phenylazetidine-1-carboxamide (I-31)

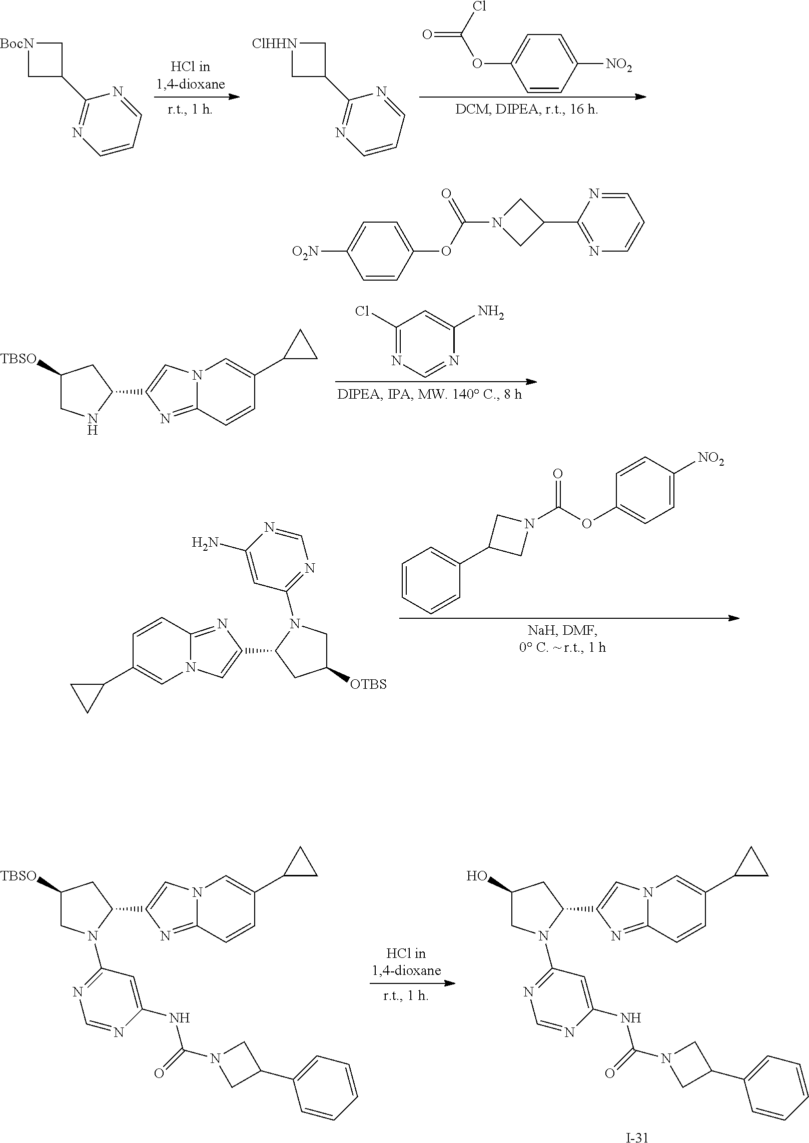

### Synthesis of 2-(azetidin-3-yl)pyrimidine hydrochloride

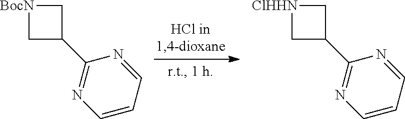

A mixture of tert-butyl 3-(pyrimidin-2-yl)azetidine-1-carboxylate (300 mg, 1.28 mmol) in HCl (4 M solution in 1,4-dioxane, 3 mL) was stirred at room temperature for 1 h. The reaction mixture was concentrated in vacuo to give 2-(azetidin-3-yl)pyrimidine hydrochloride (150 mg, crude) as a white solid. ESI-MS [M+H]⁺: 136.2.

### Synthesis of 4-nitrophenyl 3-(pyrimidin-2-yl)azetidine-1-carboxylate

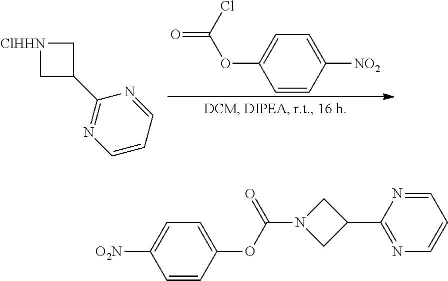

A mixture of 2-(azetidin-3-yl)pyrimidine hydrochloride (150 mg, crude), 4-nitrophenyl carbonochloridate (177 mg, 0.88 mmol), and DIPEA (568 mg, 4.4 mmol) in DCM (5 mL) was stirred at room temperature for 16 h. The reaction was concentrated in vacuo to give the crude product, which was purified by Prep-TLC (eluent: DCM/MeOH=50/1) to give 4-nitrophenyl 3-(pyrimidin-2-yl)azetidine-1-carboxylate (200 mg, 76%) as a colourless oil. ESI-MS [M+H]⁺: 301.2.

### Synthesis of 6-((2R,4S)-4-((tert-butyldimethylsilyl)oxy)-2-(6-cyclopropylimidazo[1,2-a]pyridin-2-yl)pyrrolidin-1-yl)pyrimidin-4-amine

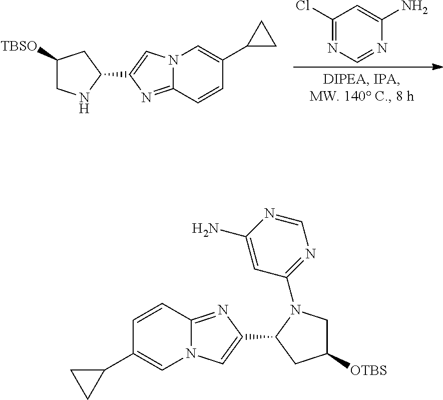 [SMILES: CC(C)(C)[Si](C)(C)O[C@@H]1CN[C@@H](c2cn3cc(C4CC4)ccc3n2)C1 ; Nc1cc(Cl)ncn1 ; CC(C)(C)[Si](C)(C)O[C@H]1C[C@H](c2cn3cc(C4CC4)ccc3n2)N(c2cc(N)ncn2)C1]

A mixture of 2-((2R, 4S)-4-((tert-butyldimethylsilyl)oxy)pyrrolidin-2-yl)-6-cyclopropylimidazo[1,2-a]pyridine (200 mg, 0.56 mmol), 6-chloropyrimidin-4-amine (72 mg, 0.56 mmol), and DIPEA (361 mg, 2.8 mmol) in i-PrOH (5 mL) was stirred in a sealed tube. After degassing with N₂ for 1 min, the reaction was irradiated in a microwave reactor at 140° C. for 8 h. The reaction was concentrated in vacuo to give the crude product, which was purified by Prep-TLC (eluent: DCM/MeOH=10/1) to give 6-((2R, 4S)-4-((tert-butyldimethylsilyl)oxy)-2-(6-cyclopropylimidazo[1,2-a]pyridin-2-yl)pyrrolidin-1-yl)pyrimidin-4-amine (160 mg, 63%) as a yellow solid. ESI-MS [M+H]⁺: 451.2.

### Synthesis of N-(6-((2R,4S)-4-((tert-butyldimethylsilyl)oxy)-2-(6-cyclopropylimidazo[1,2-a]pyridin-2-yl)pyrrolidin-1-yl)pyrimidin-4-yl)-3-phenylazetidine-1-carboxamide

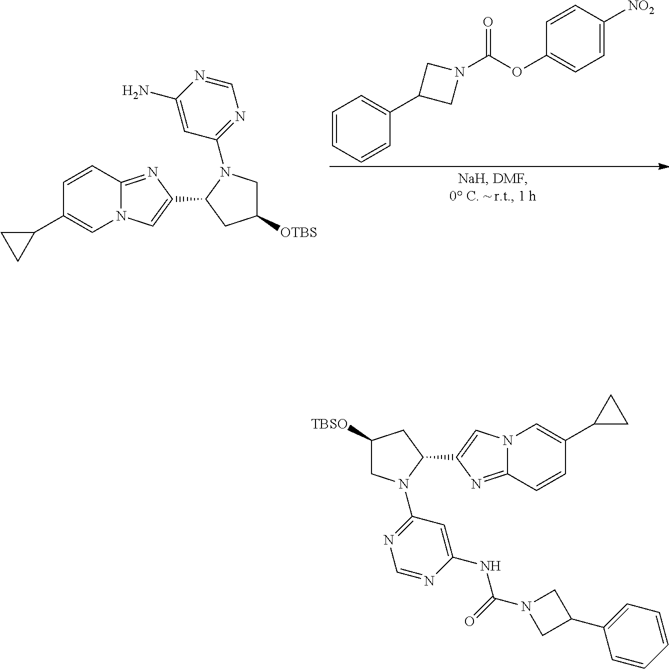 [SMILES: CC(C)(C)[Si](C)(C)O[C@H]1C[C@H](c2cn3cc(C4CC4)ccc3n2)N(c2cc(N)ncn2)C1 ; O=C(Oc1ccc([N+](=O)[O-])cc1)N1CC(c2ccccc2)C1 ; CC(C)(C)[Si](C)(C)O[C@H]1C[C@H](c2cn3cc(C4CC4)ccc3n2)N(c2cc(NC(=O)N3CC(c4ccccc4)C3)ncn2)C1]

To solution of 6-((2R, 4S)-4-((tert-butyldimethylsilyl)oxy)-2-(6-cyclopropylimidazo[1,2-a]pyridin-2-yl)pyrrolidin-1-yl)pyrimidin-4-amine (45 mg, 0.1 mmol) in dry DMF (3 mL) was added NaH (12 mg, 60% dispersion in mineral oil, 0.3 mmol) at 0° C. After the mixture was stirred at 0° C. for 0.5 h, a solution of 4-nitrophenyl 3-phenylazetidine-1-carboxylate (30 mg, 0.1 mmol) in dry DMF (2 mL) was added and the resulting mixture was stirred at room temperature for another 1 h. The reaction mixture was quenched with saturated aqueous NH₄Cl (20 mL) and extracted with EtOAc (30 mL×3). The combined organic layers were washed with brine (30 mL), dried over anhydrous Na₂SO₄, and concentrated in vacuo to give the crude, which was purified by Prep-TLC (eluent: DCM/MeOH=20/1) to give N-(6-((2R, 4S)-4-((tert-butyldimethylsilyl)oxy)-2-(6-cyclopropylimidazo[1,2-a]pyridin-2-yl)pyrrolidin-1-yl)pyrimidin-4-yl)-3-phenylazetidine-1-carboxamide (20 mg, 33%) as a yellow solid. ESI-MS [M+H]*: 610.2.

### Synthesis of N-(6-((2R,4S)-2-(6-cyclopropylimidazo[1,2-a]pyridin-2-yl)-4-hydroxypyrrolidin-1-yl)pyrimidin-4-yl)-3-phenylazetidine-1-carboxamide

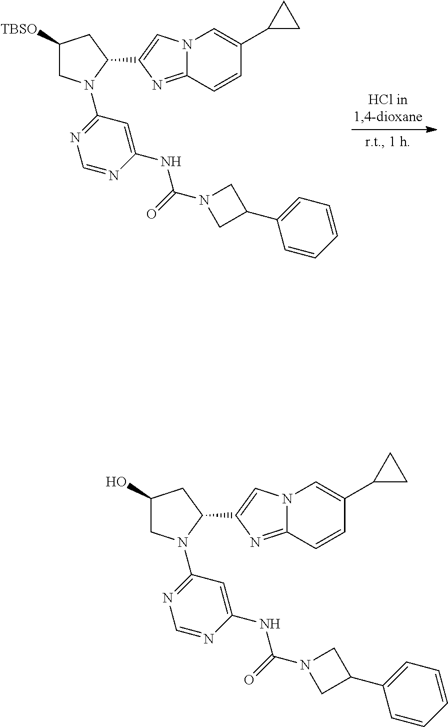 [SMILES: CC(C)(C)[Si](C)(C)O[C@H]1C[C@H](c2cn3cc(C4CC4)ccc3n2)N(c2cc(NC(=O)N3CC(c4ccccc4)C3)ncn2)C1 ; O=C(Nc1cc(N2C[C@@H](O)C[C@@H]2c2cn3cc(C4CC4)ccc3n2)ncn1)N1CC(c2ccccc2)C1]

A solution of N-(6-((2R,4S)-4-((tert-butyldimethylsilyl)oxy)-2-(6-cyclopropylimidazo[1,2-a]pyridin-2-yl)pyrrolidin-1-yl)pyrimidin-4-yl)-3-phenylazetidine-1-carboxamide (20 mg, 0.033 mmol) in HCl (4 M solution in 1,4-dioxane, 2 mL) was stirred at room temperature for 1 h. The reaction mixture was concentrated in vacuo to give the crude, which was purified by Prep-HPLC to give N-(6-((2R, 4S)-2-(6-cyclopropylimidazo[1,2-a]pyridin-2-yl)-4-hydroxypyrrolidin-1-yl)pyrimidin-4-yl)-3-phenylazetidine-1-carboxamide (5 mg, 31%) as a white solid. ESI-MS [M+H]⁺: 496.2. ¹H NMR (400 MHz, CD₃OD) δ 8.13 (s, 2H), 7.63 (s, 1H), 7.44-7.29 (m, 5H), 7.27-7.22 (m, 1H), 7.12-7.04 (m, 2H), 5.19 (s, 1H), 4.87 (s, 1H), 4.65-4.52 (m, 1H), 4.46-4.42 (m, 2H), 4.02-3.97 (m, 3H), 3.84-3.78 (m, 1H), 2.50-2.45 (m, 2H), 2.00-1.79 (m, 1H), 1.02-0.86 (m, 2H), 0.79-0.56 (m, 2H).

### Example 32

### Synthesis of 1-(4-chlorobicyclo[4.2.0]octa-1(6),2,4-trien-7-yl)-3-(6-(((6-cyclopropylimidazo[1,2-a]pyridin-2-yl)methyl)amino)pyrimidin-4-yl)urea (I-32)

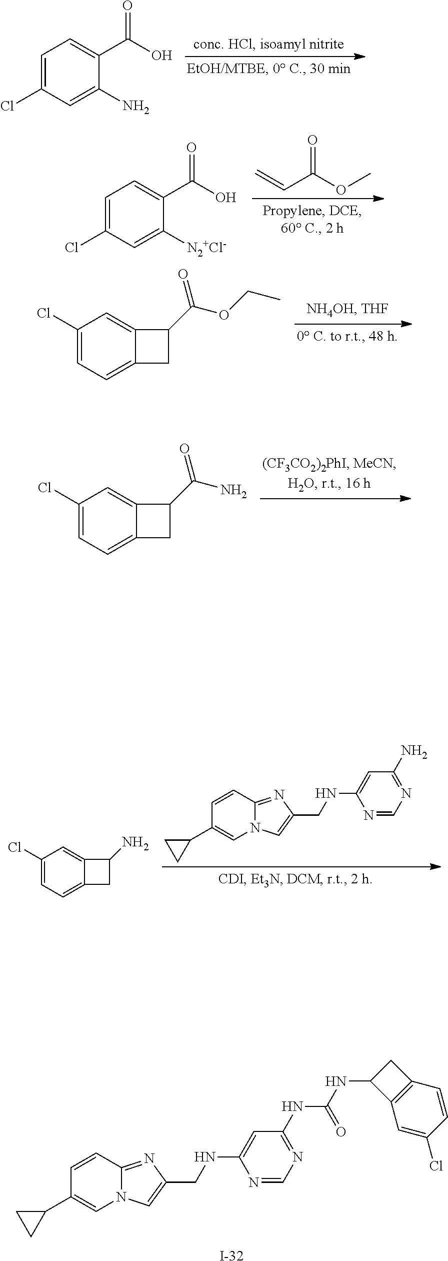

### Synthesis of 2-carboxy-5-chlorobenzenediazonium chloride

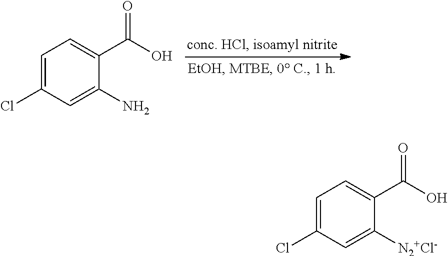

To a mixture of 2-amino-4-chlorobenzoic acid (5.0 g, 29.3 mmol) in EtOH (50 mL) and MTBE (50 mL) was added conc. HCl (20 mL) at 0° C. Then isoamyl nitrite (3.4 g, 29.3 mmol) was added at 0° C. The reaction mixture was stirred at 0° C. for 1 h. The reaction was warmed to room temperature and filtered. The precipitate was dried in vacuo to give the product 2-carboxy-5-chlorobenzenediazonium chloride (4.0 g, 63%) as a white solid.

### Synthesis of ethyl 4-chlorobicyclo[4.2.0]octa-1,3,5-triene-7-carboxylate

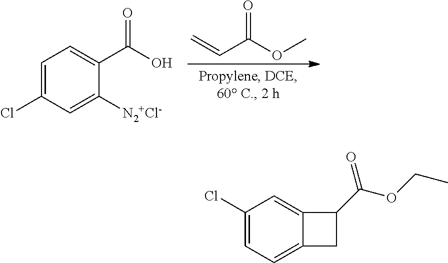

A mixture of 4-chloro-2-(chlorodiazenyl)benzoic acid (4.0 g, 18.3 mmol), methyl acrylate (7.9 g, 91.5 mmol), and propylene (3.8 g, 91.5 mmol) in 1,2-dichloroethane (50.0 mL) was stirred at 60° C. for 2 h. The reaction was concentrated in vacuo to give the crude product, which was purified by column chromatography (eluent: DCM/MeOH=15/1) to give ethyl 4-chlorobicyclo[4.2.0]octa-1,3,5-triene-7-carboxylate (1.1 g, 28%) as a colorless oil. ESI-MS [M+H]⁺: 211.2.

### Synthesis of 4-chlorobicyclo[4.2.0]octa-1,3,5-triene-7-carboxamide

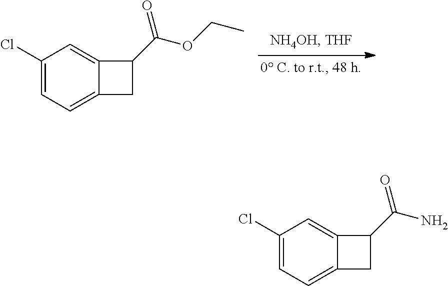

To a mixture of ethyl 4-chlorobicyclo[4.2.0]octa-1,3,5-triene-7-carboxylate (1.0 g, 4.76 mmol) in THF (20 mL) was added NH₄OH (20 mL) at 0° C. The reaction mixture was stirred at room temperature for 48 h. Water (30 mL) was added and extracted with EtOAc (50 mL×3). The combined organic layers were washed with brine (50 mL), dried over anhydrous Na₂SO₄, and concentrated in vacuo to give 4-chlorobicyclo[4.2.0]octa-1,3,5-triene-7-carboxamide (700 mg, crude) as a yellow solid, which was used in the next step without further purification. ESI-MS [M+H]⁺: 182.2.

### Synthesis of 4-chlorobicyclo[4.2.0]octa-1,3,5-trien-7-amine

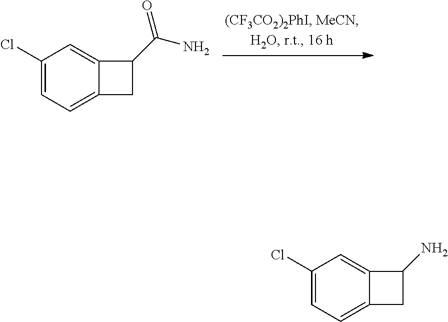 [SMILES: NC(=O)C1Cc2ccc(Cl)cc21 ; NC1Cc2ccc(Cl)cc21]

To a mixture of 4-chlorobicyclo[4.2.0]octa-1,3,5-triene-7-carboxamide (350 mg, crude) in MeCN/H₂O (10 mL/5 mL) was added (CF₃CO₂)2PhI (1.63 g, 3.32 mmol) at 0° C. The reaction mixture was stirred at room temperature for 16 h. The reaction was quenched with water (30 mL) and extracted with EtOAc (30 mL×3). The combined organic layers were washed with brine (50 mL), dried over anhydrous Na₂SO₄, and concentrated in vacuo to give the crude, which was purified by silica gel column chromatography (eluent: DCM/MeOH=20/1) to give 4-chlorobicyclo[4.2.0]octa-1,3,5-trien-7-amine (100 mg, 27%) as a yellow oil. ESI-MS [M+H]+: 154.2.

### Synthesis of 1-(4-chlorobicyclo[4.2.0]octa-1(6),2,4-trien-7-yl)-3-(6-(((6-cyclopropylimidazo[1,2-a]pyridin-2-yl)methyl)amino)pyrimidin-4-yl)urea

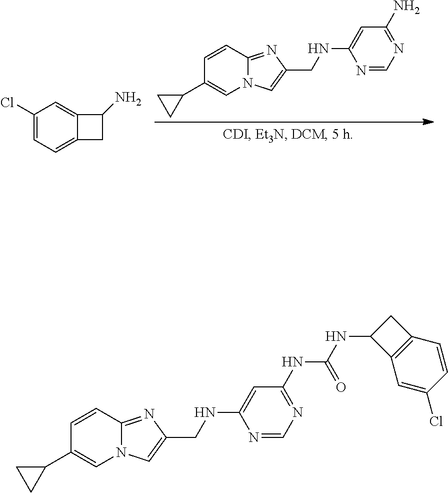

A mixture of 4-chlorobicyclo[4.2.0]octa-1,3,5-trien-7-amine (31 mg, 0.2 mmol), N⁴-((6-cyclopropylimidazo[1,2-a]pyridin-2-yl)methyl)pyrimidine-4,6-diamine (56 mg, 0.2 mmol), and CDI (65 mg, 0.4 mmol) in DCM (5 mL) was stirred at room temperature for 5 h. The reaction was quenched with water (20 mL) and extracted with DCM (30 mL×3). The combined organic layers were washed with brine (20 mL), dried over anhydrous Na₂SO₄, and concentrated in vacuo to give the crude product, which was purified by Prep-HPLC to give 1-(4-chlorobicyclo[4.2.0]octa-1(6),2,4-trien-7-yl)-3-(6-(((6-cyclopropylimidazo[1,2-a]pyridin-2-yl)methyl)amino)pyrimidin-4-yl)urea (5 mg, 5%) as a white solid. ESI-MS [M+H]⁺: 460.2. ¹H NMR (400 MHz, DMSO) δ 9.01 (s, 1H), 8.42-8.30 (m, 2H), 8.12-8.10 (m, 1H), 7.75 (s, 1H), 7.64-7.60 (m, 1H), 7.38-7.20 (m, 3H), 6.97-6.94 (m, 1H), 6.62 (s, 1H), 5.25-5.20 (m, 1H), 4.53 (s, 2H), 3.64-3.55 (m, 1H), 3.02-2.95 (m, 1H), 1.94-1.87 (m, 1H), 0.93-0.88 (m, 2H), 0.68-0.64 (m, 2H).

### Example 33

### Synthesis of N-(6-((2R,4S)-2-(6-cyclopropylimidazo[1,2-a]pyridin-2-yl)-4-hydroxypyrrolidin-1-yl)pyrimidin-4-yl)-3-(pyrimidin-2-yl)azetidine-1-carboxamide (I-33)

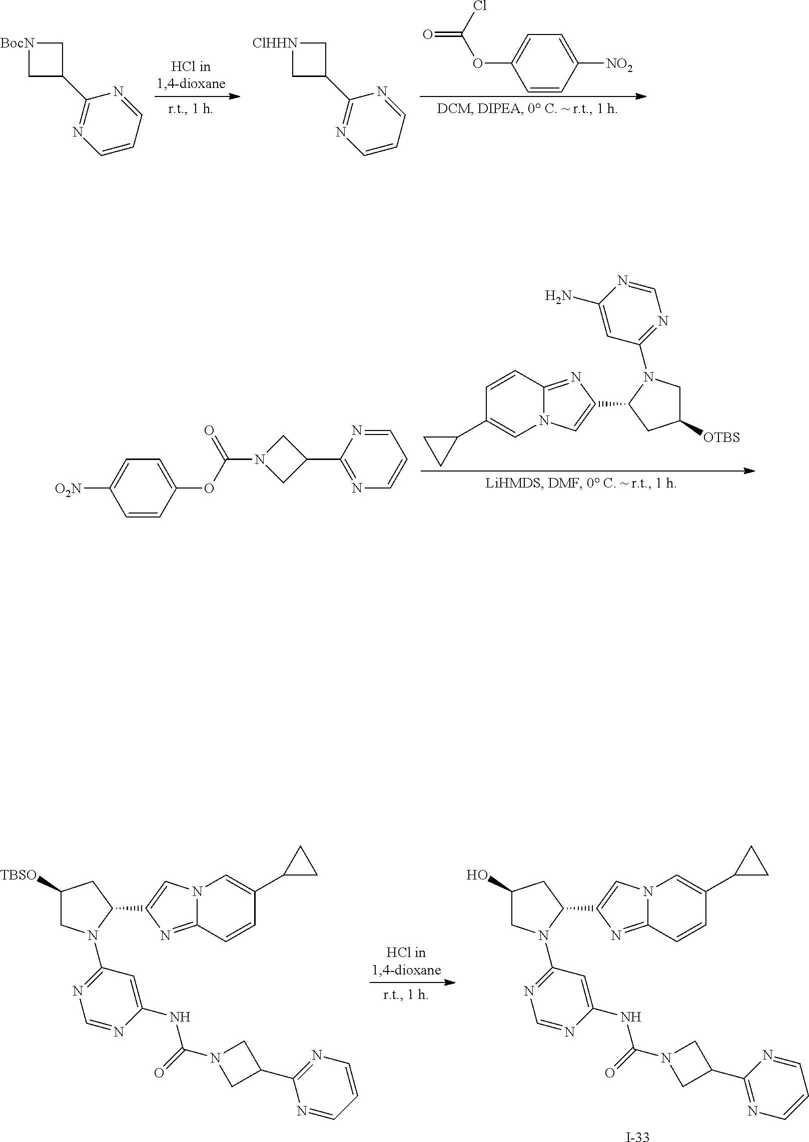

### Synthesis of 2-(azetidin-3-yl)pyrimidine hydrochloride

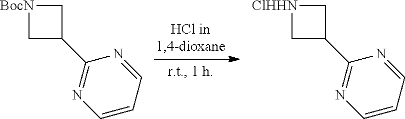

A mixture of tert-butyl 3-(pyrimidin-2-yl)azetidine-1-carboxylate (300 mg, 1.28 mmol) in HCl (4 M solution in 1,4-dioxane, 5 mL) was stirred at room temperature for 1 h. The reaction mixture was concentrated in vacuo to give 2-(azetidin-3-yl)pyrimidine as the hydrochloric acid salt (200 mg, crude) as a yellow solid. ESI-MS [M+H]⁺: 136.2.

### Synthesis of 4-nitrophenyl 3-(pyrimidin-2-yl)azetidine-1-carboxylate

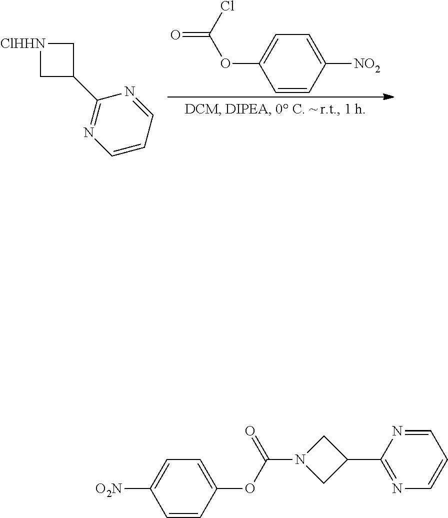

To a mixture of 2-(azetidin-3-yl)pyrimidine hydrochloride (200 mg, crude) and DIPEA (453 mg, 3.51 mmol) in DCM (10.0 mL) was added a solution of 4-nitrophenyl carbonochloridate (353 mg, 1.76 mmol) in DCM (5.0 mL) at 0° C. The reaction mixture was stirred at room temperature for 1 h. The reaction was quenched with water (20 mL) and extracted with DCM (20 mL×3). The combined organic layers were washed with brine (50 mL), dried over anhydrous Na₂SO₄, and concentrated in vacuo to give the crude, which was purified by silica gel column chromatography (eluent: DCM/MeOH=50/1) to give 4-nitrophenyl 3-(pyrimidin-2-yl)azetidine-1-carboxylate (200 mg, 52% for 2 steps) as a yellow solid. ESI-MS [M+H]⁺: 301.2.

### Synthesis of N-(6-((2R,4S)-4-((tert-butyldimethylsilyl)oxy)-2-(6-cyclopropylimidazo[1,2-a]pyridin-2-yl)pyrrolidin-1-yl)pyrimidin-4-yl)-3-(pyrimidin-2-yl)azetidine-1-carboxamide

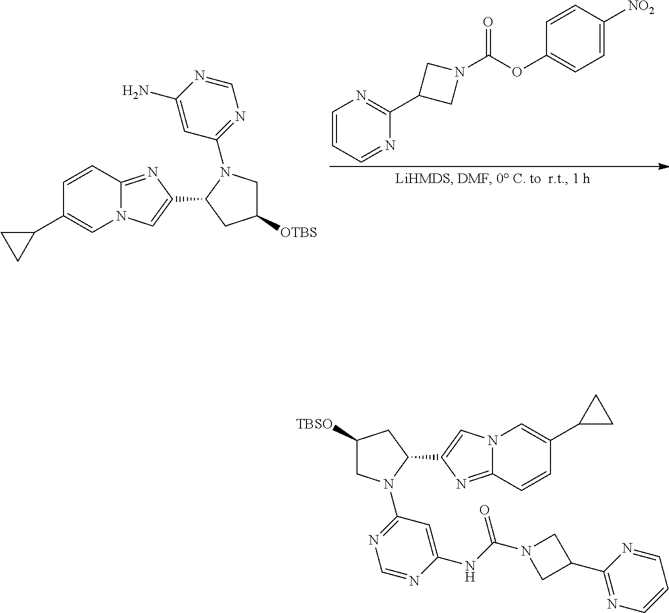 [SMILES: CC(C)(C)[Si](C)(C)O[C@H]1C[C@H](c2cn3cc(C4CC4)ccc3n2)N(c2cc(N)ncn2)C1 ; O=C(Oc1ccc([N+](=O)[O-])cc1)N1CC(c2ncccn2)C1 ; CC(C)(C)[Si](C)(C)O[C@H]1C[C@H](c2cn3cc(C4CC4)ccc3n2)N(c2cc(NC(=O)N3CC(c4ncccn4)C3)ncn2)C1]

To solution of 6-((2R, 4S)-4-((tert-butyldimethylsilyl)oxy)-2-(6-cyclopropylimidazo[1,2-a]pyridin-2-yl)pyrrolidin-1-yl)pyrimidin-4-amine (45 mg, 0.1 mmol) in dry DMF (2.0 mL) was added LiHMDS (1 M in THF, 0.3 mL) at 0° C.. The resulting mixture was stirred at 0° C. for 0.5 h. Then a solution of 4-nitrophenyl 3-(pyrimidin-2-yl)azetidine-1-carboxylate (30 mg, 0.1 mmol) in dry DMF (1 mL) was added and stirred at room temperature for another 1 h. The reaction was quenched with saturated aqueous NH₄Cl (20 mL) and extracted with EtOAc (30 mL×3). The combined organic layers were washed with brine (30 mL), dried over anhydrous Na₂SO₄, and concentrated in vacuo to give the crude, which was purified by Prep-TLC (eluent: DCM/MeOH=20/1) to give N-(6-((2R,4S)-4-((tert-butyldimethylsilyl)oxy)-2-(6-cyclopropylimidazo[1,2-a]pyridin-2-yl)pyrrolidin-1-yl)pyrimidin-4-yl)-3-(pyrimidin-2-yl)azetidine-1-carboxamide (30 mg, 49%) as a yellow solid. ESI-MS [M+H]+: 612.2.

### Synthesis of N-(6-((2R,4S)-2-(6-cyclopropylimidazo[1,2-a]pyridin-2-yl)-4-hydroxypyrrolidin-1-yl)pyrimidin-4-yl)-3-(pyrimidin-2-yl)azetidine-1-carboxamide

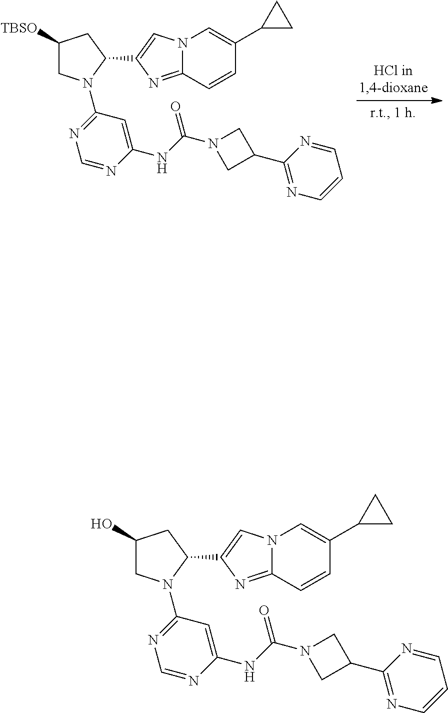 [SMILES: CC(C)(C)[Si](C)(C)O[C@H]1C[C@H](c2cn3cc(C4CC4)ccc3n2)N(c2cc(NC(=O)N3CC(c4ncccn4)C3)ncn2)C1 ; O=C(Nc1cc(N2C[C@@H](O)C[C@@H]2c2cn3cc(C4CC4)ccc3n2)ncn1)N1CC(c2ncccn2)C1]

A mixture of N-(6-((2R,4S)-4-((tert-butyldimethylsilyl)oxy)-2-(6-cyclopropylimidazo[1,2-a]pyridin-2-yl)pyrrolidin-1-yl)pyrimidin-4-yl)-3-(pyrimidin-2-yl)azetidine-1-carboxamide (30 mg, 0.05 mmol) in HCl (4 M solution in 1,4-dioxane, 2 mL) was stirred at room temperature for 1 h. The reaction mixture was concentrated in vacuo to give the crude, which was purified by Prep-HPLC to give N-(6-((2R, 4S)-2-(6-cyclopropylimidazo[1,2-a]pyridin-2-yl)-4-hydroxypyrrolidin-1-yl)pyrimidin-4-yl)-3-(pyrimidin-2-yl)azetidine-1-carboxamide (12 mg, 48%) as a white solid. ESI-MS [M+H]⁺: 498.2. ¹H NMR (400 MHz, CD₃OD) δ 8.77 (d, J=4.9 Hz, 2H), 8.13 (s, 2H), 7.62 (s, 1H), 7.37-7.33 (m, 2H), 7.17-6.96 (m, 2H), 5.16 (s, 1H), 4.59-4.56 (m, 1H), 4.54-4.21 (m, 4H), 4.20-3.84 (m, 3H), 2.44-2.43 (m, 2H), 1.99-1.83 (m, 1H), 1.05-0.83 (m, 2H), 0.78-0.51 (m, 2H).

### Example 34

### Synthesis of (4-methylpyrimidin-2-yl)methyl (6-(((6-cyclopropylimidazo[1,2-a]pyridin-2-yl)methyl)amino)pyrimidin-4-yl)carbamate (I-34)

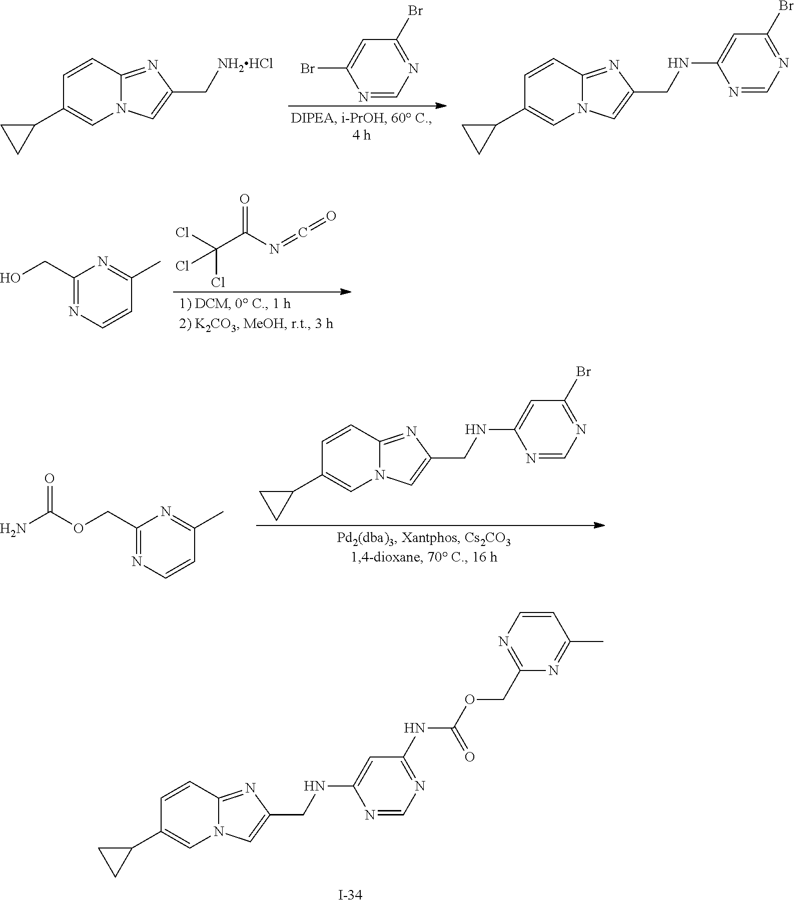

### Synthesis of 6-bromo-N-((6-cyclopropylimidazo[1,2-a]pyridin-2-yl)methyl)pyrimidin-4-amine

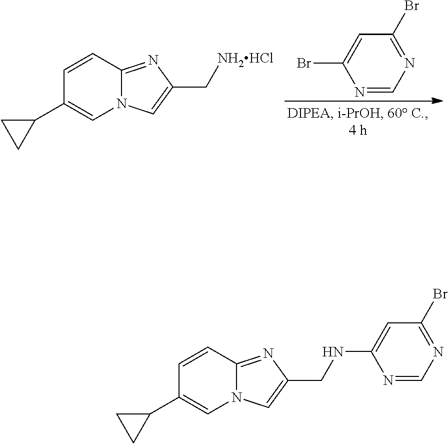

To a solution of (6-cyclopropylimidazo[1,2-a]pyridin-2-yl)methanamine hydrochloride (446 mg, 2 mmol) and 4,6-dibromopyrimidine (569 mg, 2.4 mmol) in iPrOH (10 mL) was added DIPEA (775 mg, 6 mmol). The mixture was stirred at 60° C. for 4 h. The reaction mixture was concentrated in vacuo, and the residue purified by silica gel column (eluting with 0˜-5% MeOH/DCM) to afford 6-bromo-N-((6-cyclopropylimidazo[1,2-a]pyridin-2-yl)methyl)pyrimidin-4-amine (475 mg, 69%) as a yellow solid. ESI-MS [M+H]+: 345.2.

### Synthesis of (4-methylpyrimidin-2-yl)methyl carbamate

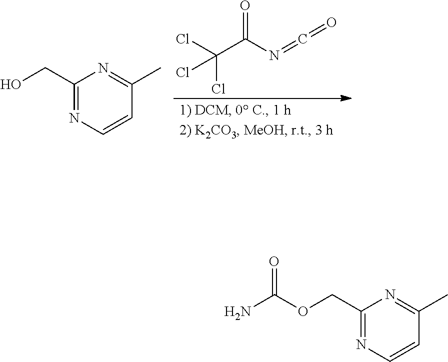

To a solution of (4-methylpyrimidin-2-yl)methanol (500 mg, 4 mmol) in DCM (15 mL) was added 2,2,2-trichloroacetyl isocyanate (1.5 g, 8 mmo) at 0° C. The resulting mixture was stirred at 0° C. for 1 h. Then the mixture was concentrated in vacuo, then re-dissolved in MeOH (15 mL). K₂CO₃ (1.67 g, 12 mmol) was added and the mixture was stirred at room temperature for 3 h. The mixture was diluted with water (50 mL) and extracted with EtOAc (20 mL×3). The combined organic layers were washed with brine (20 mL), dried over Na₂SO₄, then concentrated in vacuo to give the crude product. The crude product was purified by Prep-TLC (DCM:MeOH=10:1) to give (4-methylpyrimidin-2-yl)methyl carbamate (150 mg, 22.5% yield) as a white solid. ESI-MS [M+H]+: 168.1.

### Synthesis of (4-methylpyrimidin-2-yl)methyl (6-(((6-cyclopropylimidazo[1,2-a]pyridin-2-yl)methyl)amino)pyrimidin-4-yl)carbamate

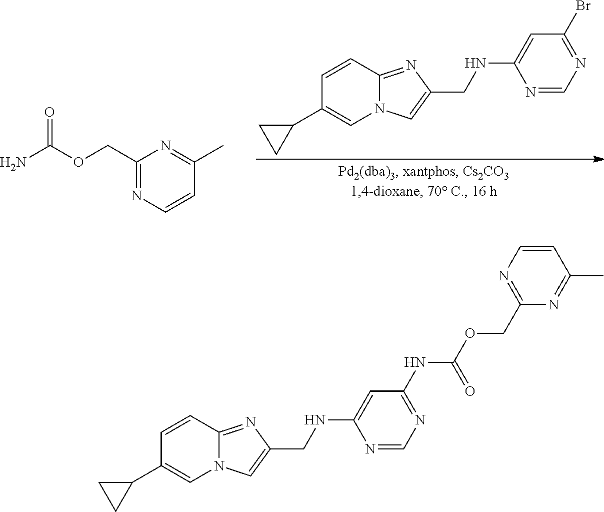

A mixture of (4-methylpyrimidin-2-yl)methyl carbamate (100 mg, 0.60 mmol), 6-bromo-N-((6-cyclopropylimidazo[1,2-a]pyridin-2-yl)methyl)pyrimidin-4-amine (162 mg, 0.47 mmol), Pd₂(dba)₃ (110 mg, 0.12 mmol), Xantphos (139 mg, 0.24 mmol), and Cs₂CO₃ (587 mg, 1.8 mmol) in 1,4-dioxane (15 mL) was stirred at 70° C. for 16 h under N₂. After cooling to room temperature, the mixture was filtered through Celite® and the filter cake was washed with DCM/MeOH (10/1, 50 mL). The filtrate was concentrated in vacuo to give the crude, which was purified by Prep-HPLC to give (4-methylpyrimidin-2-yl)methyl (6-(((6-cyclopropylimidazo[1,2-a]pyridin-2-yl)methyl)amino)pyrimidin-4-yl)carbamate (29 mg, 14% yield) as a white solid. ESI-MS [M+H]+: 431.2, ¹H NMR (400 MHz, DMSO) δ 10.27 (s, 1H), 8.62 (d, J=5.1 Hz, 1H), 8.32-8.26 (m, 1H), 8.17 (s, 1H), 7.78 (s, 1H), 7.59 (s, 1H), 7.38-7.29 (m, 2H), 6.99-6.91 (m, 2H), 5.23 (s, 2H), 4.55 (s, 2H), 2.45 (s, 3H)1.96-1.85 (m, 1H), 0.95-0.85 (m, 2H), 0.69-0.61 (m, 2H).

### Example 35

Inhibitory Activity of Exemplary Compounds against Plasma Kallikrein.

Compounds were evaluated for inhibition of the human activated kallikrein enzyme in two formats of an assay employing a fluorogenic peptide substrate. In one assay format, the concentrations of reagents were as follows: 20 mM Tris pH 7.5, 1 mM EDTA, 150 mM sodium chloride, 0.1% PEG-400, 0.1% Triton X-100, 500 pM activated kallikrein enzyme, 300 uM Pro-Phe-Arg-7-amido-4-methylcoumarin (PFR-AMC) substrate. Prior to reaction initiation with substrate, enzyme and inhibitors were preincubated for 30 min at room temperature. After initiation with substrate, reactions were incubated for 10 min at room temperature and fluorescence emission at 460 nm from 380 nm excitation measured with a microplate reader. In another assay format, the concentrations of reagents were as follows: 20 mM Tris pH 7.5, 1 mM EDTA, 150 mM sodium chloride, 0.1% PEG-400, 0.1% Triton X-100, 5 pM activated kallikrein enzyme, 300 uM PFR-AMC substrate. Prior to reaction initiation with substrate, enzyme and inhibitors were preincubated for 30 min at room temperature. After initiation with substrate, reactions were incubated for 18 hr at room temperature and fluorescence emission at 460 nm from 380 nm excitation measured with a microplate reader.

Table 2 provides the results of the assay in the format with 500 pM activated kallikrein assay. For the compounds listed in Table 2, the EC₅₀ values are reported according to the following ranges: A≤5.0 nM; 5.0 nM<B≤50 nM; 50 nM<C≤500 nM; 500 nM<D≤9000 nM; 9000 nM<E.

TABLE 2

| | Primary Dose |
|---|---|
| | Response |
| | Assay: |
| Compound | Average EC50 |
| I-1 | B |
| I-2 | A |
| I-3 | D |
| I-4 | C |
| I-5 | D |
| I-6 | C |
| I-7 | B |
| I-8 | A |
| I-9 | B |
| I-10 | A |
| I-11 | A |
| I-12 | A |
| I-13 | A |
| I-14 | C |
| I-15 | A |
| I-16 | C |
| I-17 | C |
| I-18 | E |
| I-19 | B |
| I-20 | C |
| I-21 | A |
| I-22 | A |
| I-23 | A |
| I-24 | D |
| I-25 | E |
| I-26 | A |
| I-27 | C |
| I-28 | A |
| I-29 | C |
| I-30 | D |
| I-31 | D |
| I-32 | D |
| I-33 | C |
| I-34 | B |

### Example 36

Neat Human and Rat Plasma Assay

To analyze inhibition of plasma kallikrein in an ex vivo setting, the potency of compounds was measure in contact pathway-activated plasma assays. In a fluorogenic peptide substrate assay, test compounds dissolved in DMSO were added to sodium citrate collected human or rat plasma in a 96-well microplate. Alternatively, citrated plasma was collected from rats administered the compounds orally or by IV. 10 nM of human FXIIa (Enzyme Research Laboratories) diluted in PKa buffer (20 mM Tris-HCl, pH 7.5, 150 mM NaCl, 1 mM EDTA, 0.1% PEG-8000, and 0.1% Triton X-100) was added to the plasma, followed by the 100 μM of the profluorescent, synthetic plasma kallikrein substrate PFR-AMC (also diluted in PKa buffer). Final plasma concentration in the reaction was 78%. Fluorescence was immediately monitored by excitation/emission wavelengths of 360 nm/480 nm respectively over a period of 5 minutes in a microplate reader. The resulting linear increase in fluorescence emission (reflecting PKa proteolysis of PFR-AMC substrate) was fit to extract a proteolytic rate (fluorescent units over time), and this rate was subsequently plotted against compound inhibitor concentration. Resulting plots were fit to a standard 4-parameter IC50/IC90 equation to determine min/max values, IC50/90, and slope. All experimental steps were performed at room temperature. Table 3 provides results of the assay.

For the compounds listed in Table 3, the IC₉₀ values are reported according to the following ranges: A≤2500 nM; 2500 nM<B≤7500 nM; 7500 nM<C≤15000 nM.

TABLE 3

| | Human |
|---|---|
| | Plasma |
| Compound | Assay: IC90 |
| I-8 | A |
| I-10 | B |
| I-11 | B |
| I-12 | C |
| I-13 | C |
| I-15 | B |
| I-21 | B |
| I-26 | A |
| I-28 | A |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of examples.

### EXEMPLARY ENUMERATED EMBODIMENTS

1. A compound of Formula I:

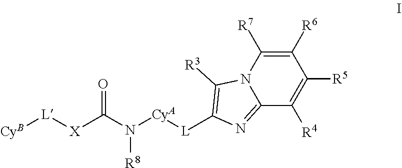

or a pharmaceutically acceptable salt thereof, wherein

Cy^{A} is a phenylene or 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4 —R^{A} groups;
each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur;

each R is independently hydrogen or an optionally substituted C₁₋₆ aliphatic group;

Cy^{B} is selected from phenyl, 8- to 10-membered bicyclic aryl, 5- to 6-membered heteroaryl having 1-3 heteroatoms independently selected from oxygen, nitrogen, and sulfur or 7- to 10-membered heteroaryl having 1-4 heteroatoms independently selected from oxygen, nitrogen, and sulfur, wherein Cy^{B} is substituted with 0-5 —R^{B} groups;
each R^{B} is independently selected from oxo, halogen, haloalkyl, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;

L is an optionally substituted C₁₋₃ hydrocarbon chain, wherein 1 to 3 methylene units are optionally and independently replaced with —O—, —NR^{z}—, —S—, —SO—, or —SO₂—; or L is an optionally substituted 5- to 6-membered saturated or partially unsaturated heterocyclene, having 1-3 heteroatoms independently selected from oxygen, nitrogen, and sulfur;
each R^{z} is independently selected from hydrogen, —(CH₂)₀₋₃OR, —(CH₂)₀₋₃C(O)OR, or an optionally substituted C₁₋₆ aliphatic group;

X is —O— or —NR^{y}—;

R^{y} is hydrogen or an optionally substituted C₁₋₆ aliphatic group;

L′ is a covalent bond or an optionally substituted C₁₋₃ hydrocarbon chain, wherein a carbon of L′ may optionally be taken together with R^{y} to form a 3- to 7-membered heterocyclic ring;

each R³, R⁴, R⁵, R⁶, and R⁷ is independently selected from hydrogen or -L^{C}-R^{C}, wherein
each L^{C} is independently selected from a covalent bond or an optionally substituted C₁₋₆ hydrocarbon chain, wherein 1 to 3 methylene units are optionally and independently replaced with —O— or —NR—;
each R^{C} is independently selected from halogen, haloalkyl, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{C}, or an optionally substituted group selected from C₁₋₆ aliphatic;
each Cy^{C} is independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{C} is substituted with 0-4 -L^{D}-R^{D} groups;
   each L^{D} is independently selected from a covalent bond or an optionally substituted C₁₋₆ hydrocarbon chain, wherein 1 to 3 methylene units are optionally and independently replaced with —O— or —NR—;
   each R^{D} is independently selected from oxo, halogen, haloalkyl, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur; and

R⁸ is selected from hydrogen, —OR, or an optionally substituted C₁₋₆ aliphatic group.

2. The compound of embodiment 1, wherein Cy^{A} is a 5- to 6-membered monocyclic heteroarylene having 1-3 heteroatoms independently selected from oxygen, nitrogen, and sulfur, wherein Cy^{A} is substituted with 0-4 —R^{A} groups.
3. The compound of embodiment 1 or 2, wherein Cy^{A} is a 6-membered monocyclic heteroarylene having 1-3 heteroatoms independently selected from oxygen, nitrogen, and sulfur, wherein Cy^{A} is substituted with 0-4 —R^{A} groups.
4. The compound of any one of embodiments 1-3, wherein Cy^{A} is selected from the group consisting of:

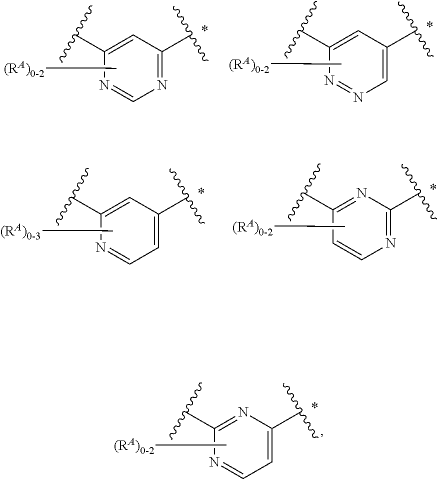

wherein * represents the point of attachment to L.
5. The compound of any one of embodiments 1-3, wherein Cy^{A} is a pyridinediyl substituted with 0-1 R^{A} groups.
6. The compound of any one of embodiments 1-3, wherein Cy^{A} is a pyrimidinediyl substituted with 0-1 R^{A} groups.
7. The compound of embodiment 1, wherein Cy^{A} is a 7- to 12-membered bicyclic heteroarylene having 1-4 heteroatoms independently selected from oxygen, nitrogen, and sulfur, wherein Cy^{A} is substituted with 0-4 —R^{A} groups.
8. The compound of embodiment 7, wherein Cy^{A} is triazolopyridinediyl, imidazopyridinediyl, or triazolopyrazinediyl, wherein Cy^{A} is substituted with 0-1 —R^{A} groups.
9. The compound of any one of the preceding embodiments, wherein Cy^{B} is selected from phenyl and a 5- to 6-membered heteroaryl having 1-3 heteroatoms independently selected from oxygen, nitrogen, and sulfur, wherein Cy^{B} is substituted with 0-4 —R^{B} groups.
10. The compound of any one of the preceding embodiments, wherein Cy^{B} is phenyl, wherein Cy^{B} is substituted with 0-3 —R^{B} groups.
11. The compound of any one embodiments 1-9, wherein Cy^{B} is a 6-membered heteroaryl having 1-3 nitrogens, wherein Cy^{B} is substituted with 0-4 —R^{B} groups.
12. The compound of any one of the preceding embodiments, wherein Cy^{B} is a pyrimidinyl group substituted with 0-2 —R^{B} groups. In some embodiments, Cy^{B} is a pyridinyl group substituted with 0-2 —R^{B} groups.
13. The compound of any one of the preceding embodiments, wherein Cy^{B} is selected from the group consisting of:

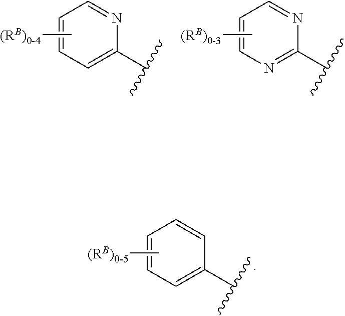

14. The compound of any one of the preceding embodiments, wherein each R^{B} is independently selected from halogen, —OR, or an optionally substituted group selected from C₁₋₆ aliphatic or a 5-membered heteroaryl having 1-4 heteroatoms independently selected from oxygen, nitrogen, and sulfur.
15. The compound of any one of the preceding embodiments, wherein L is an optionally substituted C₁₋₃ hydrocarbon chain, wherein 1 methylene unit is optionally replaced with —O— or —NR^{z}—.
16. The compound of any one of the preceding embodiments, wherein L is an optionally substituted C₂ hydrocarbon chain, wherein 1 methylene unit is optionally replaced with —NR^{z}— or —O—.
17. The compound of any one of the preceding embodiments, wherein L is an optionally substituted C₂ hydrocarbon chain, wherein the methylene unit connected to Cy^{A} is replaced with —NR^{z}— or —O—.
18. The compound of any one of the preceding embodiments, wherein L is an optionally substituted C₂ hydrocarbon chain, wherein the methylene unit connected to Cy^{A} is replaced with —NR^{z}—.
19. The compound of any one of embodiments 1-17, wherein L is an optionally substituted C₂ hydrocarbon chain, wherein the methylene unit connected to Cy^{A} is replaced with —O—.
20. The compound of embodiment 18, wherein L is *—NHCH₂—, wherein * represents the point of attachment to Cy^{A}.
21. The compound of embodiment 19, wherein L is *—OCH₂—, wherein * represents the point of attachment to Cy^{A}.
22. The compound of any one of embodiments 1-14, wherein L is an optionally substituted 5-membered saturated or partially unsaturated heterocyclene, having 1 heteroatom independently selected from oxygen, nitrogen, and sulfur. In some embodiments, L is an optionally substituted pyrrolidinediyl group.
23. The compound of any one of embodiments 1-14 or 22, wherein L is

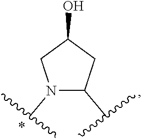 [SMILES: *C1C[C@H](O)CN1*]

wherein * represents the point of attachment to Cy^{A}.
24. The compound of any one of the preceding embodiments, wherein L comprises a two-atom spacer between Cy^{A} and

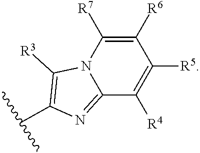

25. The compound of any one of the preceding embodiments, wherein X is —O—.
26. The compound of any one of the preceding embodiments, wherein X is —NR^{y}—.
27. The compound of embodiment 26, wherein R^{y} is hydrogen.
28. The compound of embodiment 26, wherein R^{y} is an optionally substituted C₁₋₆ aliphatic group.
29. The compound of any one of the preceding embodiments, wherein L′ is an optionally substituted C₁₋₃ hydrocarbon chain.
30. The compound of any one of the preceding embodiments, wherein L′ is —CH(CH₃)—.
31. The compound of any one of the preceding embodiments, wherein L′ is

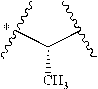

wherein * represents the point of attachment to Cy^{B}.
32. The compound of any one of the preceding embodiments, wherein L′ is a methylene unit optionally substituted with —(CH₂)₀₋₄R^{∘} or —(CH₂)₀₋₄OR^{∘}, wherein R^{∘} is hydrogen or C₁₋₆ aliphatic.
33. The compound of any one embodiments 1-28, wherein L′ is CH₂.
34. The compound of any one of embodiments 1-28, wherein a carbon of L′ may optionally be taken together with R^{z} to form a 3- to 7-membered heterocyclic ring.
35. The compound of embodiment 34, wherein a carbon of L′ is taken together with R^{y} to form a 4-membered heterocyclic ring.
36. The compound of any one of the preceding embodiments, wherein R³ is hydrogen.
37. The compound of any one of the preceding embodiments, wherein R⁴ is hydrogen
38. The compound of any one of the preceding embodiments, wherein R⁵ is hydrogen.
39. The compound of any one of the preceding embodiments, wherein R⁶ is selected from hydrogen or L^{C}-R^{C}, wherein L^{C} is a covalent bond, and wherein R^{C} is selected from halogen, —N(R)₂, —OR, Cy^{C}, or an optionally substituted C₁₋₆ aliphatic group.
40. The compound of embodiment 37, wherein Cy^{C} is a 3- to 7-membered saturated or partially unsaturated monocyclic carbocyclyl substituted with 0-4 L^{D}-R^{D} groups.
41. The compound of embodiment 38, wherein Cy^{C} is a cyclopropyl group substituted with 0-4 L^{D}-R^{D} groups.
42. The compound of any one of the preceding embodiments, wherein Cy^{C} is an unsubstituted cyclopropyl group.
43. The compound of any one of the preceding embodiments, wherein R⁷ is hydrogen.
44. The compound of any one of the preceding embodiments, wherein R⁸ is hydrogen.
45. The compound of any one of the preceding embodiments, wherein the compound is of Formulae II-a or II-b:

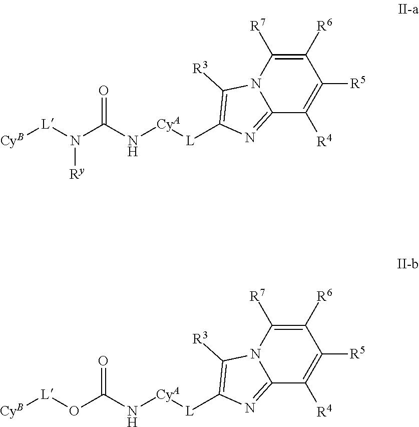

or a pharmaceutically acceptable salt thereof.
46. The compound of any one of the preceding embodiments, wherein the compound is of Formulae III-a-1, III-b-1, III-a-2, III-b-2, III-a-3, or III-b-3:

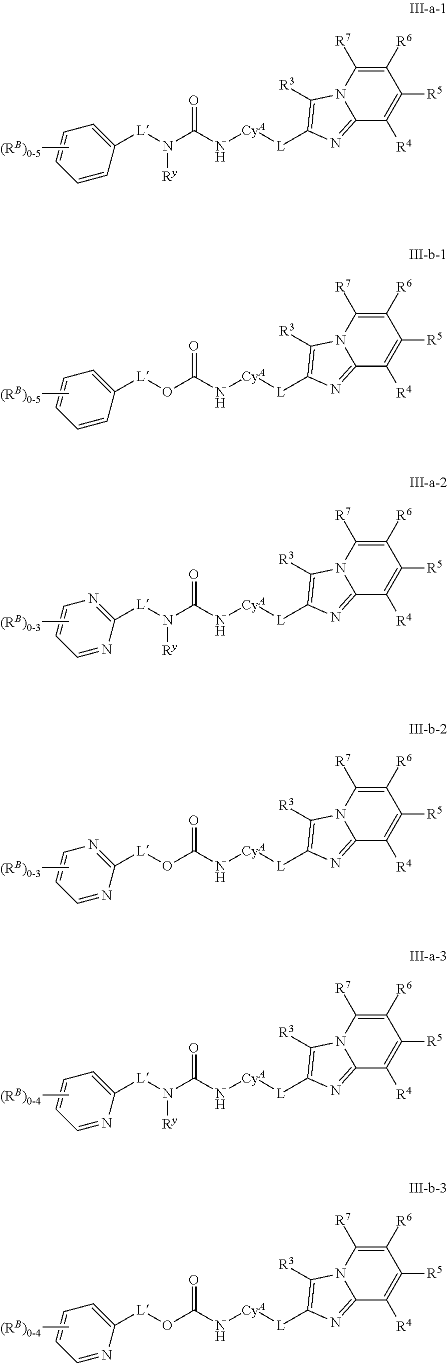

or a pharmaceutically acceptable salt thereof.
47. The compound of any one of the preceding embodiments, wherein the compound is of Formulae IV-a-1, IV-b-1, IV-a-2, or IV-b-2:

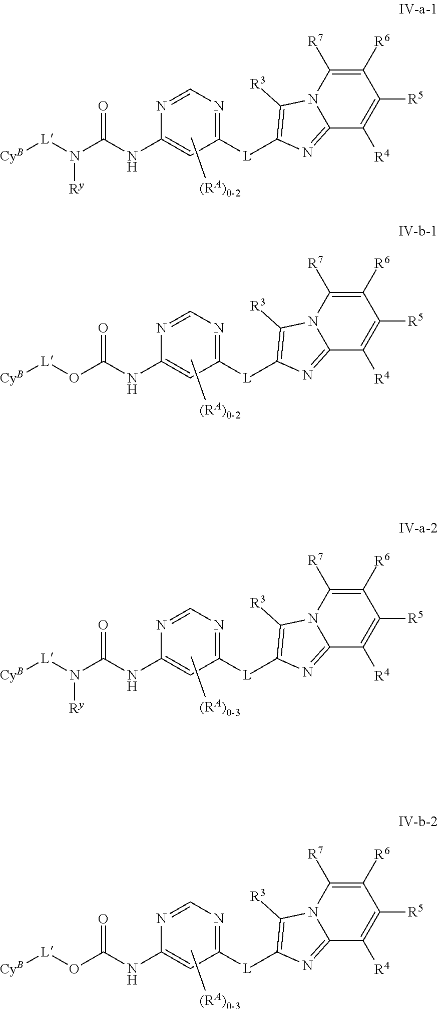

or a pharmaceutically acceptable salt thereof.
48. The compound of any one of the preceding embodiments, wherein the compound is of Formula V:

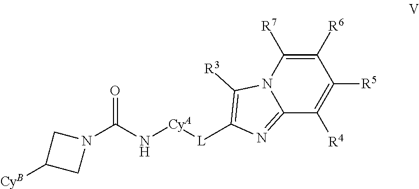

or a pharmaceutically acceptable salt thereof.
49. The compound of any one of the preceding embodiments, wherein the compound is of Formulae VI-a, VI-b, or VI-c:

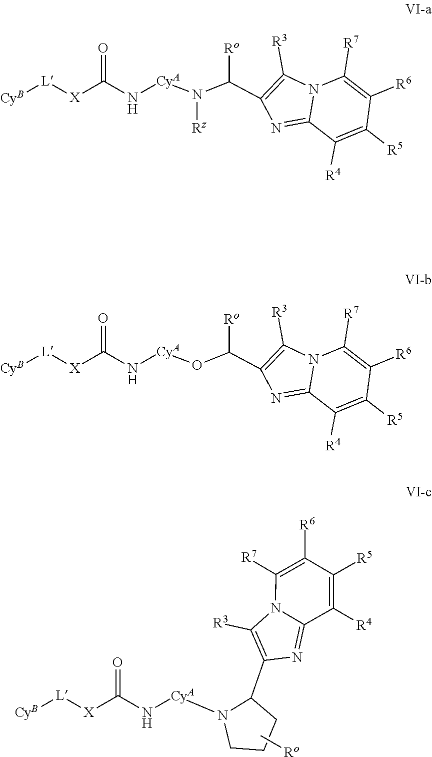

or a pharmaceutically acceptable salt thereof.
50. The compound of any one of the preceding embodiments, wherein the compound is selected from compounds I-1 through I-34, or a pharmaceutically acceptable salt thereof.
51. A pharmaceutical composition comprising a compound of any one of the preceding embodiments.
52. The pharmaceutical composition comprising a compound of any one of the preceding embodiments, further comprising a pharmaceutically acceptable excipient.
53. The composition of embodiment 51 or 52, wherein the composition is suitable for oral administration.
54. A method of treating a plasma kallikrein-mediated disease or disorder using a compound or composition of any one of the preceding embodiments.
55. The method of embodiment 54, wherein the disease or disorder is hereditary angioedema.
56. The method of embodiment 54, wherein the disease or disorder is diabetic macular edema.
57. A method of treating hereditary angioedema comprising administering to a patient in need thereof a compound or composition of any one of the preceding embodiments.
58. A method of treating diabetic macular edema comprising administering to a patient in need thereof a compound or composition of any one of the preceding embodiments.
59. The method of any one of embodiments 55 or 57, wherein administration of the compound partially or completely inhibits, delays onset of, reduces severity of, and/or reduces incidence of one or more symptoms, features, and/or causes of a hereditary angioedema.
60. The method of embodiment 59, wherein the compound is administered orally.

1. A compound of Formula I: 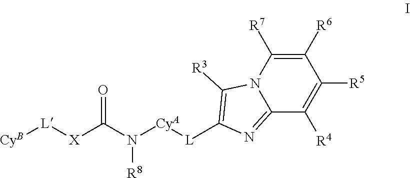
or a pharmaceutically acceptable salt thereof, wherein
Cy^{A} is a phenylene or 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4—R^{A} groups;
each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur;
each R is independently hydrogen or an optionally substituted C₁₋₆ aliphatic group;
Cy^{B} is selected from phenyl, 8- to 10-membered bicyclic aryl, 5- to 6-membered heteroaryl having 1-3 heteroatoms independently selected from oxygen, nitrogen, and sulfur or 7- to 10-membered heteroaryl having 1-4 heteroatoms independently selected from oxygen, nitrogen, and sulfur, wherein Cy^{B} is substituted with 0-5 —R^{B} groups;
each R^{B} is independently selected from oxo, halogen, haloalkyl, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L is an optionally substituted C₁₋₃ hydrocarbon chain, wherein 1 to 3 methylene units are optionally and independently replaced with —O—, —NR^{z}—, —S—, —SO—, or —SO₂—; or L is an optionally substituted 5- to 6-membered saturated or partially unsaturated heterocyclene, having 1-3 heteroatoms independently selected from oxygen, nitrogen, and sulfur;
each R^{z} is independently selected from hydrogen, —(CH₂)₀₋₃OR, —(CH₂)₀₋₃C(O)OR, or an optionally substituted C₁₋₆ aliphatic group;
X is —O— or —NR^{y}—;
R^{y} is hydrogen or an optionally substituted C₁₋₆ aliphatic group;
L′ is a covalent bond or an optionally substituted C₁₋₃ hydrocarbon chain, wherein a carbon of L′ may optionally be taken together with R^{y} to form a 3- to 7-membered heterocyclic ring;
each R³, R⁴, R⁵, R⁶, and R⁷ is independently selected from hydrogen or -L^{C}-R^{C}, wherein
each L^{C} is independently selected from a covalent bond or an optionally substituted C₁₋₆ hydrocarbon chain, wherein 1 to 3 methylene units are optionally and independently replaced with —O— or —NR—;
each R^{C} is independently selected from halogen, haloalkyl, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{C}, or an optionally substituted group selected from C₁₋₆ aliphatic;
each Cy^{C} is independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{C} is substituted with 0-4 -L^{D}-R^{D} groups;
each L^{D} is independently selected from a covalent bond or an optionally substituted C₁₋₆ hydrocarbon chain, wherein 1 to 3 methylene units are optionally and independently replaced with —O— or —NR—;
each R^{D} is independently selected from oxo, halogen, haloalkyl, —CN, —C(O)R, —C(O)₂R, —C(O)N(R)₂, —NO₂, —N(R)₂, —N(R)C(O)R, —N(R)C(O)₂R, —N(R)S(O)₂R,
—OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur; and
R⁸ is selected from hydrogen, —OR, or an optionally substituted C₁₋₆ aliphatic group.

2. The compound of claim 1, wherein Cy^{A} is a 5- to 6-membered monocyclic heteroarylene having 1-3 heteroatoms independently selected from oxygen, nitrogen, and sulfur, wherein Cy^{A} is substituted with 0-4—R^{A} groups.

3. The compound of claim 1, wherein Cy^{A} is selected from the group consisting of: 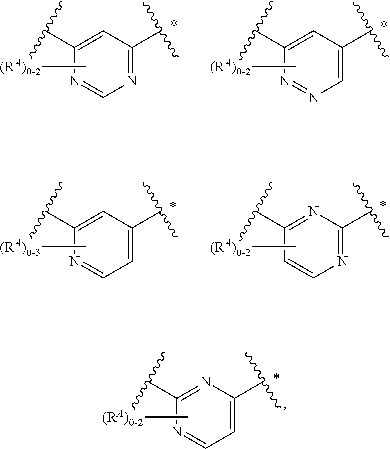
wherein * represents the point of attachment to L.

4. The compound of claim 1, wherein Cy^{A} is a 7- to 12-membered bicyclic heteroarylene having 1-4 heteroatoms independently selected from oxygen, nitrogen, and sulfur, wherein Cy^{A} is substituted with 0-4—R^{A} groups.

5. The compound of claim 1, wherein Cy^{A} is triazolopyridinediyl, imidazopyridinediyl, or triazolopyrazinediyl, wherein Cy^{A} is substituted with 0-1 —R^{A} groups.

6. The compound of claim 1, wherein Cy^{B} is selected from phenyl and a 5- to 6-membered heteroaryl having 1-3 heteroatoms independently selected from oxygen, nitrogen, and sulfur, wherein Cy^{B} is substituted with 0-4 —R^{B} groups.

7. The compound of claim 1, wherein Cy^{B} is selected from the group consisting of:

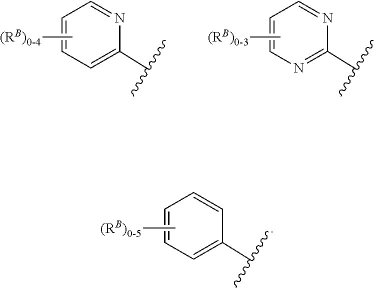

8. The compound of claim 1, wherein each R^{B} is independently selected from halogen, —OR, or an optionally substituted group selected from C₁₋₆ aliphatic or a 5-membered heteroaryl having 1-4 heteroatoms independently selected from oxygen, nitrogen, and sulfur.

9. The compound of claim 1, wherein L is an optionally substituted C₁₋₃ hydrocarbon chain, wherein 1 methylene unit is optionally replaced with —O— or —NR^{z}—.

10. The compound of claim 1, wherein L is *—NHCH₂— or *—OCH₂—, wherein * represents the point of attachment to Cy^{A}.

11. The compound of claim 1, wherein L is an optionally substituted 5-membered saturated or partially unsaturated heterocyclene, having 1 heteroatom independently selected from oxygen, nitrogen, and sulfur.

12. The compound of claim 1, wherein L′ is an optionally substituted C₁₋₃ hydrocarbon chain.

13. The compound of claim 1, wherein L′ is, —CH(CH₃)—, CH₂, 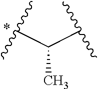
wherein * represents the point of attachment to Cy^{B}.

14. The compound of claim 1, wherein a carbon of L′ may optionally be taken together with R^{z} to form a 3- to 7-membered heterocyclic ring.

15. The compound of claim 1, wherein a carbon of L′ is taken together with R^{y} to form a 4-membered heterocyclic ring.

16. The compound of claim 1, wherein R³ is hydrogen.

17. The compound of claim 1, wherein R⁴ is hydrogen

18. The compound of claim 1, wherein R⁵ is hydrogen.

19. The compound of claim 1, wherein R⁶ is selected from hydrogen or L^{C}-R^{C}, wherein L^{C} is a covalent bond, and wherein R^{C} is selected from halogen, —N(R)₂, —OR, Cy^{C}, or an optionally substituted C₁₋₆ aliphatic group.

20. The compound of claim 1, wherein Cy^{C} is an unsubstituted cyclopropyl group.

21. The compound of claim 1, wherein R⁷ is hydrogen.

22. The compound of claim 1, wherein the compound is of Formulae II-a or II-b: 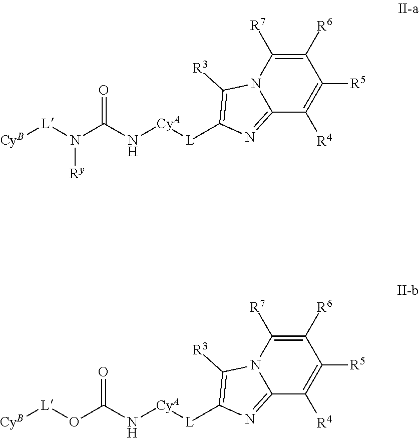
or a pharmaceutically acceptable salt thereof.

23. The compound of claim 1, wherein the compound is of Formulae III-a-1, III-b-1, III-a-2, III-b-2, III-a-3, or III-b-3: 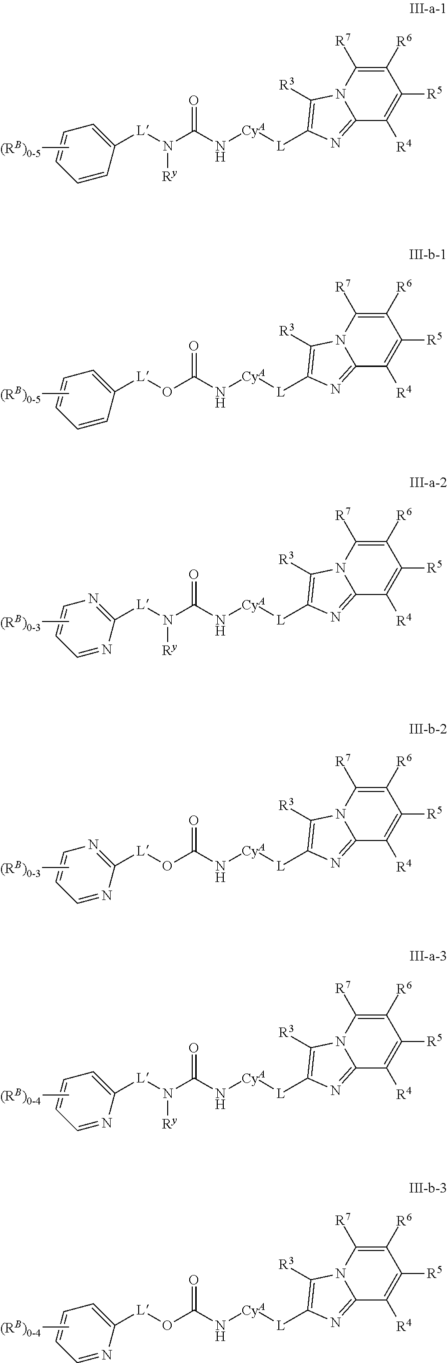
or a pharmaceutically acceptable salt thereof.

24. The compound of claim 1, wherein the compound is of Formulae IV-a-1, IV-b-1, IV-a-2, or IV-b-2: 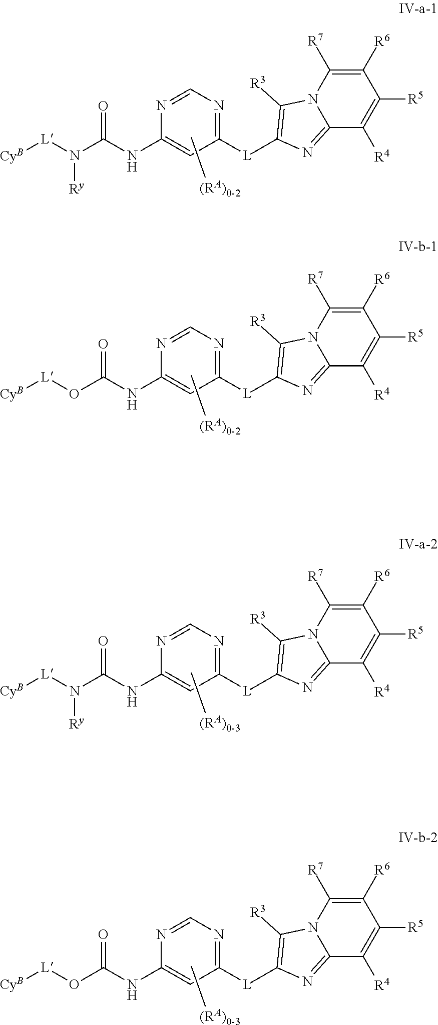
or a pharmaceutically acceptable salt thereof.

25. The compound of claim 1, wherein the compound is of Formula V: 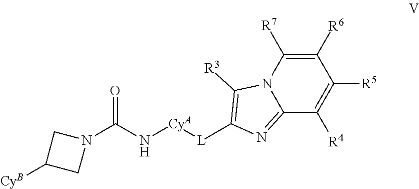
or a pharmaceutically acceptable salt thereof.

26. The compound of claim 1, wherein the compound is of Formulae VI-a, VI-b, or VI-c: 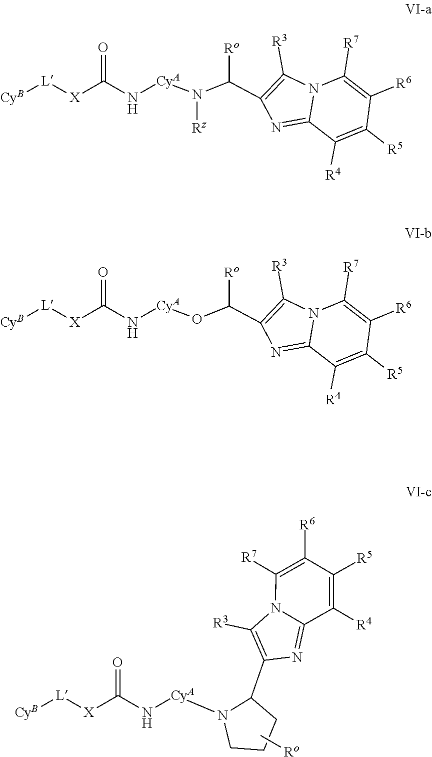
or a pharmaceutically acceptable salt thereof.

27. The compound of claim 1, wherein the compound is selected from compounds I-1 through I-34, or a pharmaceutically acceptable salt thereof.

28. A pharmaceutical composition comprising a compound of claim 1.

29. A method of treating a plasma kallikrein-mediated disease or disorder using a compound of claim 1.

30. The method of claim 29, wherein the disease or disorder is hereditary angioedema or diabetic macular edema.
